SEQUENCE LISTING (57 sequences)

SEQ ID NO: 1 - protein, 112 aa, Artificial Sequence
  Light chain variable domain sequence for Anti- LAG-3 antibody A6 clone
DVVMTQSPLPLPVTPGEPASITCRSSQSLLHSNGYNYLDWYLQKPGQSPQLLIYLGSNRASGVPDRFSGSGSGTDFTLKISRVEAEDVGVYYCMQALQTPYTFGQGTKLEIK

SEQ ID NO: 2 - protein, 107 aa, Artificial Sequence
  Light chain variable domain sequence for Anti- LAG-3 antibody 1G11 clone
EIVLTQSPGTLSLSPGERATLSCRASQSVSSSFLAWYQQKPGQAPRLLIYGASSRATGIPDRFSGSGSGTDFTLTISRLEPEDFAVYYCQQYGPSITFGGGTKVEIK

SEQ ID NO: 3 - protein, 109 aa, Artificial Sequence
  Light chain variable domain sequence for Anti- LAG-3 antibody C2 clone
EIVMTQSPGTLSLSPGERATLSCRASQSVSSSYLAWYQQKPGQAPRLLIYGASSRATGIPDRFSGSGSGTDFTLTISRLEPEDFAVYYCQQYGSSPPITFGQGTRLEIK

SEQ ID NO: 4 - protein, 112 aa, Artificial Sequence
  Light chain variable domain sequence for Anti- LAG-3 antibody C12 clone
DVVMTQSPLSLPVTPGEPASISCRSSQSLLHSDGYNYFDWYLQKPGQSPQLLIYLGSNRAAGVPDRFSGSGSGTDFTLKISRVEAEDVGVYYCMQGTHWPPTFGQGTKLEIK

SEQ ID NO: 5 - protein, 110 aa, Artificial Sequence
  Light chain variable domain sequence for Anti- LAG-3 antibody F5 clone
ETTLTQSPGTLSLSPGERATLSCRASQSVSSGYLAWYQQKPGQAPRLLIYDASSRATGIPDRFSGSGSGADFTLTISRLQPEDFAVYYCQQYGSSRPGLTFGGGTRVEIK

SEQ ID NO: 6 - protein, 108 aa, Artificial Sequence
  Light chain variable domain sequence for Anti- LAG-3 antibody G8 clone
EIVLTQSPGTLSLSPGERATLSCTTSQSVSSTSLDWYQQKPGQAPRLLIYGASSRATGIPDRFSGSGSGTDFTLTISRLEPEDFAVYYCQQYGSSLLTFGGGTKVEIK

SEQ ID NO: 7 - protein, 116 aa, Artificial Sequence
  Heavy chain variable domain sequence for Anti- LAG-3 antibody A6 clone
EVQLVQSGAEVKKPGSSVKVSCKASGYTFTSYYMHWVRQAPGQGLEWMGIINPSGGSTSYAQKFQGRVTMTRDTSTSTVYMELSSLRSEDTAVYYCAMPFGDFDYWGQGTLVTVSS

SEQ ID NO: 8 - protein, 120 aa, Artificial Sequence
  Heavy chain variable domain sequence for Anti- LAG-3 antibody 1G11 clone
QLQLQESGGDVVQPGRSLRLSCAASGFTFSSYGMHWVRQAPGKGLEWVAVISYDGSNKYYADSVKGRFTISRDNSKNTLYLQMNSLRAEDTAVYYCARLPGWGAYAFDIWGQGTMVTVSS

SEQ ID NO: 9 - protein, 126 aa, Artificial Sequence
  Heavy chain variable domain sequence for Anti- LAG-3 antibody C2 and G8 clone
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARDPDAANWGFLLYYGMDVWGQGTTVTVSS

SEQ ID NO: 10 - protein, 126 aa, Artificial Sequence
  Heavy chain variable domain sequence for Anti- LAG-3 antibody C12 clone
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARALADFWSGYYYYYYMDVWGKGTTVTVSS

SEQ ID NO: 11 - protein, 117 aa, Artificial Sequence
  Heavy chain variable domain sequence for Anti- LAG-3 antibody F5 clone
EVQLVQSGAEVKKPGASVKVSCKVSGYTLTELSMHWVRQTPGKGLEWMGGFDPEDGETIYAQKFQGRVTMTEDTSTDTAYMELSSLRSEDTAVYYCATTWFGELYYWGQGTLVTVSS

SEQ ID NO: 12 - protein, 16 aa, Artificial Sequence
  A6 clone LC-CDR1
RSSQSLLHSNGYNYLD

SEQ ID NO: 13 - protein, 7 aa, Artificial Sequence
  A6 clone LC-CDR2
LGSNRAS

SEQ ID NO: 14 - protein, 9 aa, Artificial Sequence
  A6 clone LC-CDR3
MQALQTPYT

SEQ ID NO: 15 - protein, 12 aa, Artificial Sequence
  1G11 clone LC-CDR1
RASQSVSSSFLA

SEQ ID NO: 16 - protein, 7 aa, Artificial Sequence
  1G11, C2 and G8 clone LC-CDR2
GASSRAT

SEQ ID NO: 17 - protein, 8 aa, Artificial Sequence
  1G11 clone LC-CDR3
QQYGPSIT

SEQ ID NO: 18 - protein, 12 aa, Artificial Sequence
  C2 clone LC-CDR1
RASQSVSSSYLA

SEQ ID NO: 19 - protein, 10 aa, Artificial Sequence
  C2 clone LC-CDR3
QQYGSSPPIT

SEQ ID NO: 20 - protein, 16 aa, Artificial Sequence
  C12 clone LC-CDR1
RSSQSLLHSDGYNYFD

SEQ ID NO: 21 - protein, 7 aa, Artificial Sequence
  C12 clone LC-CDR2
LGSNRAA

SEQ ID NO: 22 - protein, 9 aa, Artificial Sequence
  C12 clone LC-CDR3
MQGTHWPPT

SEQ ID NO: 23 - protein, 12 aa, Artificial Sequence
  F5 clone LC-CDR1
RASQSVSSGYLA

SEQ ID NO: 24 - protein, 7 aa, Artificial Sequence
  F5 clone LC-CDR2
DASSRAT

SEQ ID NO: 25 - protein, 11 aa, Artificial Sequence
  F5 clone LC-CDR3
QQYGSSRPGLT

SEQ ID NO: 26 - protein, 12 aa, Artificial Sequence
  G8 clone LC-CDR1
TTSQSVSSTSLD

SEQ ID NO: 27 - protein, 9 aa, Artificial Sequence
  G8 clone LC-CDR3
QQYGSSLLT

SEQ ID NO: 28 - protein, 5 aa, Artificial Sequence
  A6 clone HC-CDR1
SYYMH

SEQ ID NO: 29 - protein, 17 aa, Artificial Sequence
  A6 clone HC-CDR2
IINPSGGSTSYAQKFQG

SEQ ID NO: 30 - protein, 7 aa, Artificial Sequence
  A6 clone HC-CDR3
PFGDFDY

SEQ ID NO: 31 - protein, 5 aa, Artificial Sequence
  1G11 clone HC-CDR1
SYGMH

SEQ ID NO: 32 - protein, 17 aa, Artificial Sequence
  1G11, C2 and G8 clones HC-CDR2
VISYDGSNKYYADSVKG

SEQ ID NO: 33 - protein, 11 aa, Artificial Sequence
  1G11 HC-CDR3
LPGWGAYAFDI

SEQ ID NO: 34 - protein, 5 aa, Artificial Sequence
  C2 and G8 clones HC-CDR1
SYAMH

SEQ ID NO: 35 - protein, 17 aa, Artificial Sequence
  C2 and G8 clones HC-CDR3
DPDAANWGFLLYYGMDV

SEQ ID NO: 36 - protein, 9 aa, Artificial Sequence
  C12 clone HC-CDR1
GTFSSYAIS

SEQ ID NO: 37 - protein, 17 aa, Artificial Sequence
  C12 clone HC-CDR2
GIIPIFGTANYAQKFQG

SEQ ID NO: 38 - protein, 17 aa, Artificial Sequence
  C12 clone HC-CDR3
ALADFWSGYYYYYYMDV

SEQ ID NO: 39 - protein, 5 aa, Artificial Sequence
  F5 clone HC-CDR1
ELSMH

SEQ ID NO: 40 - protein, 17 aa, Artificial Sequence
  F5 clone HC-CDR2
GFDPEDGETIYAQKFQG

SEQ ID NO: 41 - protein, 8 aa, Artificial Sequence
  F5 clone HC-CDR3
TWFGELYY

SEQ ID NO: 42 - DNA, 336 bp, Artificial Sequence
  A6 clone nucleotide light chain variable domain
gatgttgtgatgactcagtctccactccccctgcccgtcactcctggagagccggcctccatcacctgcaggtccagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaaccccctacacttttggccaggggaccaagctggagatcaaa

SEQ ID NO: 43 - DNA, 321 bp, Artificial Sequence
  1G11 clone nucleotide light chain variable domain
gaaattgtgttgacgcagtctccaggcaccctgtctttgtctccaggggaaagagccacgctctcctgcagggccagtcagagcgttagcagcagcttcttggcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtccctcaatcactttcggcggagggaccaaggtagagatcaaa

SEQ ID NO: 44 - DNA, 327 bp, Artificial Sequence
  C2 clone nucleotide light chain variable domain
gaaattgtgatgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctccgatcaccttcggccaagggacacgactggagattaaa

SEQ ID NO: 45 - DNA, 336 bp, Artificial Sequence
  C12 clone nucleotide light chain variable domain
gatgttgtgatgactcagtctccactctccctgcccgtcacccctggagagccggcctccatctcctgcaggtctagtcagagcctcctgcatagtgatggatacaactatttcgattggtacctgcagaagccagggcagtctccacagctcctgatctatttgggttctaatcgggccgccggggtccctgacaggttcagtggcagtggatcaggcacagattttacactgaaaatcagcagagtggaggctgaggatgttggggtttattactgcatgcaaggtacacactggcctcccacttttggccaggggaccaagctggagatcaaa

SEQ ID NO: 46 - DNA, 330 bp, Artificial Sequence
  F5 clone nucleotide light chain variable domain
gaaacgacactcacgcagtctccaggcaccctgtctttgtctccaggggaaagagccaccctctcctgcagggccagtcagagtgttagcagcggctacttagcctggtaccagcagaaacctggccaggctcccaggctcctcatctatgatgcatccagcagggccactggcatcccagacaggttcagtggcagtgggtctggggcagacttcactctcaccatcagcagactacagcctgaagattttgcagtgtattactgtcaacagtatggtagttcacgtccagggctcactttcggcggagggaccagggtggagatcaaa

SEQ ID NO: 47 - DNA, 324 bp, Artificial Sequence
  G8 clone nucleotide light chain variable domain
gaaattgtgttgacgcagtctccaggcaccctgtctttgtctccaggggaaagagccaccctctcctgcacgaccagtcagagtgttagcagcacctccttagactggtaccagcagaaacctggccaggctcccaggctcctcatctatggtgcatctagcagggccactggcatcccagacaggttcagtggcagtgggtctgggacagacttcactctcaccatcagcagactggagcctgaagattttgcagtgtattactgtcagcagtatggtagctcacttctcactttcggcggagggaccaaggtggagatcaaa

SEQ ID NO: 48 - DNA, 348 bp, Artificial Sequence
  A6 clone nucleotide heavy chain variable domain
gaggtgcagctggtgcagtctggggctgaggtgaagaagcctgggtcctcggtgaaggtctcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgatgccattcggagactttgactactggggccagggaaccctggtcaccgtctcaagc

SEQ ID NO: 49 - DNA, 360 bp, Artificial Sequence
  1G11 clone nucleotide heavy chain variable domain
cagctgcagctgcaggagtcggggggagacgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaggctaccgggctggggcgcttatgcttttgatatctggggccaagggacaatggtcaccgtctcaagc

SEQ ID NO: 50 - DNA, 378 bp, Artificial Sequence
  C2 and G8 clones nucleotide heavy chain variable domain
caggtgcagctggtgcagtctgggggaggcgtggtccagcctgggaggtccctgagactctcctgtgcagcgtctggattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagctgaggacacggctgtgtattactgtgcgagagatcccgacgcggctaactggggattcttgttgtactacggtatggacgtctggggccaagggaccacggtcaccgtctcaagc

SEQ ID NO: 51 - DNA, 378 bp, Artificial Sequence
  C12 clone nucleotide heavy chain variable domain
caggtccagctggtacagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagagctctggccgatttttggagtggttactactactactactacatggacgtctggggcaaagggaccacggtcaccgtctcaagc

SEQ ID NO: 52 - DNA, 351 bp, Artificial Sequence
  F5 clone nucleotode heavy chain variable domain
gaggtccagctggtgcagtctggggctgaggtgaagaagcctggggcctcagtgaaggtctcctgcaaggtttccggatacaccctcactgaattatccatgcactgggtgcgacagactcctggaaaagggcttgagtggatgggaggttttgatcctgaagatggtgaaacaatctacgcacagaagttccagggcagagtcaccatgaccgaggacacatctacagacacagcctacatggagctgagcagcctgagatctgaggacacggccgtgtattactgtgcaaccacatggttcggggagttatattactggggccagggcaccctggtcaccgtctcaagc

SEQ ID NO: 53 - protein, 16 aa, Artificial Sequence
  Light chain CDR1 consensus sequence
  MISC_FEATURE (1)..(1): Xaa is R or T
  MISC_FEATURE (2)..(2): Xaa is S, A or T
  MISC_FEATURE (6)..(6): Xaa is L or V
  MISC_FEATURE (7)..(7): Xaa is L or S
  MISC_FEATURE (8)..(8): Xaa is H or S
  MISC_FEATURE (9)..(9): Xaa is S, G or T
  MISC_FEATURE (10)..(10): Xaa is N, F, Y, D or S
  MISC_FEATURE (11)..(11): Xaa is G or L
  MISC_FEATURE (12)..(12): Xaa is Y, A or D
  MISC_FEATURE (13)..(13): Xaa is absent or N
  MISC_FEATURE (14)..(14): Xaa is absent or Y
  MISC_FEATURE (15)..(15): Xaa is absent, Lor F
  MISC_FEATURE (16)..(16): Xaa is absent or D
XXSQSXXXXXXXXXXX

SEQ ID NO: 54 - protein, 7 aa, Artificial Sequence
  Light Chain CDR2 consensus sequence
  MISC_FEATURE (1)..(1): Xaa is L, G or D
  MISC_FEATURE (2)..(2): Xaa is G or A
  MISC_FEATURE (4)..(4): Xaa is N or S
  MISC_FEATURE (7)..(7): Xaa is S, T or A
XXSXRAX

SEQ ID NO: 55 - protein, 11 aa, Artificial Sequence
  Light chain CDR3 consensus sequence
  MISC_FEATURE (1)..(1): Xaa is M or Q
  MISC_FEATURE (3)..(3): Xaa is A, Y or G
  MISC_FEATURE (4)..(4): Xaa is L, G or T
  MISC_FEATURE (5)..(5): Xaa is Q, P, S or H
  MISC_FEATURE (6)..(6): Xaa is T, S or W
  MISC_FEATURE (7)..(7): Xaa is P, I, R or L
  MISC_FEATURE (8)..(8): Xaa is Y, T, P or L
  MISC_FEATURE (9)..(9): Xaa is absent, T, I or G
  MISC_FEATURE (10)..(10): Xaa is absent, T or L
  MISC_FEATURE (11)..(11): Xaa is absent or T
XQXXXXXXXXX

SEQ ID NO: 56 - protein, 5 aa, Artificial Sequence
  Heavy chain CDR1 consensus sequence
  MISC_FEATURE (1)..(1): Xaa is S or E
  MISC_FEATURE (2)..(2): Xaa is Y or L
  MISC_FEATURE (3)..(3): Xaa is Y, G, A or S
  MISC_FEATURE (4)..(4): Xaa is M or I
  MISC_FEATURE (5)..(5): Xaa is H or S
XXXXX

SEQ ID NO: 57 - protein, 17 aa, Artificial Sequence
  Heavy chain CDR2 consensus sequence
  MISC_FEATURE (1)..(1): Xaa is I, G or V
  MISC_FEATURE (2)..(2): Xaa is I or F
  MISC_FEATURE (3)..(3): Xaa is N, S, I or D
  MISC_FEATURE (4)..(4): Xaa is P or Y
  MISC_FEATURE (5)..(5): Xaa is S, D, I or E
  MISC_FEATURE (6)..(6): Xaa is G, F or D
  MISC_FEATURE (7)..(7): Xaa is G or S
  MISC_FEATURE (8)..(8): Xaa is S, N, T or E
  MISC_FEATURE (9)..(9): Xaa is T, K or A
  MISC_FEATURE (10)..(10): Xaa is S, Y, N or I
  MISC_FEATURE (13)..(13): Xaa is Q or D
  MISC_FEATURE (14)..(14): Xaa is K or S
  MISC_FEATURE (15)..(15): Xaa is F or V
  MISC_FEATURE (16)..(16): Xaa is Q or K
XXXXXXXXXXYAXXXXG

(19) United States
(12) Patent Application Publication
(10) Pub. No.: US 2019/0040136 A1
(43) Pub. Date: Feb. 7, 2019

(54) ANTI-LAG-3 ANTIBODIES

(71) Applicant: Agency for Science, Technology and Research, Connexis (SG)

(72) Inventors: Cheng-I WANG, Singapore (SG); Hsueh Ling Janice OH, Singapore (SG); Siok Ping YEO, Singapore (SG); Chia Yin LEE, Singapore (SG)

(21) Appl. No.: 16/079,947

(22) PCT Filed: Mar. 3, 2017
(86) PCT No.: PCT/EP2017/055060

§ 371 Date: Aug. 24, 2018

(30) Foreign Application Priority Data
Mar. 4, 2016 (SG) 10201601719R

(51) Int. Cl. C07K 16/28; C12N 15/62; A61P 35/00; A61P 31/00

(52) U.S. Cl. CPC C07K 16/2812; C12N 15/62; A61P 35/00; A61P 31/00; C07K 2317/92; C07K 2317/33; C07K 2317/76; C07K 2317/55; C07K 2317/21

(57) ABSTRACT

Anti-LAG-3 antibodies are disclosed. Also disclosed are compositions comprising such antibodies, and uses and methods using the same.

### RELATED APPLICATIONS

This application is a 35 U.S.C. § 371 national phase application of PCT/EP2017/055060 (WO 2017/149143) filed on Mar. 3, 2017 entitled “ANTI-LAG-3 ANTIBODIES”, which application claims the benefit of Singapore Application Serial No. 10201601719R, filed Mar. 4, 2016, both of which are incorporated herein by reference in their entirety.

Incorporated by reference herein in its entirety is the Sequence Listing entitled “Sequence listing.txt”, created Aug. 21, 2018, size of 30 kilobytes.

### FIELD OF THE INVENTION

The present invention relates to antibodies that bind to lymphocyte-activation gene 3 (LAG-3).

### BACKGROUND TO THE INVENTION

T-cell exhaustion is a state of T-cell dysfunction that arises during many chronic infections and cancer. It is defined by poor T-cell effector function, sustained expression of inhibitory receptors and a transcriptional state distinct from that of functional effector or memory T-cells. Exhaustion prevents optimal control of infection and tumors. (E John Wherry, Nature Immunology 12, 492-499 (2011)).

T-cell exhaustion is characterized by the stepwise and progressive loss of T-cell functions. Exhaustion is well-defined during chronic lymphocytic choriomeningitis virus (LCMV) infection and commonly develops under conditions of antigen-persistence, which occur following many chronic infections including hepatitis B virus, hepatitis C virus and human immunodeficiency virus infections, as well as during tumor metastasis. Exhaustion is not a uniformly disabled setting as a gradation of phenotypic and functional defects can manifest, and these cells are distinct from prototypic effector, memory and also anergic T cells. Exhausted T cells most commonly emerge during high-grade chronic infections, and the levels and duration of antigenic stimulation are critical determinants of the process. (Yi et al., Immunology April 2010; 129(4):474-481).

Circulating human tumor-specific CD8⁺ T cells may be cytotoxic and produce cytokines in vivo, indicating that self- and tumor-specific human CD8⁺ T cells can reach functional competence after potent immunotherapy such as vaccination with peptide, incomplete Freund's adjuvant (IFA), and CpG or after adoptive transfer. In contrast to peripheral blood, T-cells infiltrating tumor sites are often functionally deficient, with abnormally low cytokine production and upregulation of the inhibitory receptors PD-1, CTLA-4, TIM-3 and LAG-3. Functional deficiency is reversible, since T-cells isolated from melanoma tissue can restore IFN-γ production after short-term in vitro culture. However, it remains to be determined whether this functional impairment involves further molecular pathways, possibly resembling T-cell exhaustion or anergy as defined in animal models. (Baitsch et al., J Clin Invest. 2011; 121(6):2350-2360).

Lymphocyte-activation gene 3 (LAG-3), also called CD223, is a type I transmembrane protein encoded in humans by the LAGS gene. The molecular properties and biological functions of LAG-3 described herein are reviewed in Sierro et al., Expert Opin Ther Targets (2011) 15(1): 91-101. LAG-3 is a CD4-like protein, expressed at the surface of T cells (especially activated T cells) natural killer cells, B cells and plasmacytoid dendritic cells. LAG-3 has been shown to be a negative costimulatory receptor, i.e. an inhibitory receptor.

LAG-3 binds to MHC class II molecules, a family of molecules constitutively expressed at high levels at the surface of antigen presenting cells (APCs) such dendritic cells, macrophages and B cells. LAG-3 function is dependent on binding to MHC class II and signalling through its cytoplasmic domain.

LAG-3 is a negative regulator of T cell responses; inhibition of LAG-3 results in improved T cell proliferation, whilst overexpression of LAG-3 impairs antigen-driven T cell proliferation.

Crosslinking of LAG-3 on T cells impair TCR-mediated activation of CD4+ T cells, resulting in reduced proliferation, lower IL-2 production and reduced production of TH1-type cytokines (i.e. IFNγ, TNFα). LAG-3 expression is also characteristic of CD4+ CD25+ FoxP3+ regulatory T cells (Tregs). LAG-3 is expressed at high levels on CD8+ T cells following antigen stimulation, and LAG-3 expression on CD8+ T cells is similarly associated with enhanced regulatory activity and lower proliferative potential.

Studies have shown that exhausted CD8+ T cells following chronic viral infections express multiple inhibitory receptors (such as PD-1, CD160 and 2B4). LAG-3 is expressed at high levels after LCMV infection, and blockade of the PD-1/PD-L1 pathway combined with blockade of LAG-3 has been shown to dramatically reduce viral load in chronically infected mice (Blackburn et al. Nat Immunol (2009) 10:29-37). Combined inhibition of the PD-1/PD-L1 pathway and LAG-3 blockade has also been shown to provide anti-tumour efficacy (Jing et al. Journal for ImmunoTherapy of Cancer (2015) 3:2).

### SUMMARY OF THE INVENTION

The present invention is concerned with antibodies, or antigen binding fragments, that bind to LAG-3. Heavy and light chain polypeptides are also disclosed. The antibodies, antigen binding fragments and polypeptides may be provided in isolated and/or purified form and may be formulated into compositions suitable for use in research, therapy and diagnosis.

In some embodiments the antibody, or antigen binding fragment, or polypeptide may be effective to restore T-cell function in T-cells, e.g. CD4⁺ or CD8⁺ T-cells, exhibiting T-cell exhaustion or T-cell anergy.

In one aspect of the present invention an antibody, or antigen binding fragment, is provided, the amino acid sequence of the antibody may comprise the amino acid sequences i) to iii), or the amino acid sequences iv) to vi), or preferably the amino acid sequences i) to vi):

i) LC-CDR1:

(SEQ ID NO: 53)

X₁X₂SQSX₃X₄X₅X₆X₇X₈X₉X₁₀X₁₁X₁₂X₁₃;
ii) LC-CDR2:

(SEQ ID NO: 54)

X₁₄X₁₅SX₁₆RAX₁₇;
iii) LC-CDR3:

(SEQ ID NO: 55)

X₁₈QX₁₉X₂₀X₂₁X₂₂X₂₃X₂₄X₂₅X₂₆X₂₇;
iv) HC-CDR1:

(SEQ ID NO: 56)

X₂₈X₂₉X₃₀X₃₁X₃₂;
v) HC-CDR2:

(SEQ ID NO: 57)

X₃₃X₃₄X₃₅X₃₆X₃₇X₃₈X₃₉X₄₀X₄₁X₄₂YAX₄₃X₄₄X₄₅X₄₆G;
vi) HC-CDR3:

(SEQ ID NO: 30)

one of PFGDFDY,

(SEQ ID NO: 33)

LPGWGAYAFDI,

(SEQ ID NO: 35)

DPDAANWGFLLYYGMDV,

(SEQ ID NO: 38)

ALADFWSGYYYYYYMDV,
or

(SEQ ID NO: 41)

TWFGELYY;

or a variant thereof in which one or two or three amino acids in one or more of the sequences (i) to (vi) are replaced with another amino acid, where X₁=R or T; X₂=S, A or T; X₃=L or V; X₄=L or S; X₅=H or S; X₆=S, G or T; X₇=N, F, Y, D or S; X₈=G or L; X₉=Y, A or D; X₁₀=absent or N; X₁₁=absent or Y; X₁₂=absent, L or F; X₁₃=absent (i.e. no amino acid) or D; X₁₄=L, G or D; X₁₅=G or A; X₁₆=N or S; X₁₇=S, T or A; X₁₈=M or Q; X₁₉=A, Y or G; X₂₀=L, G or T; X₂₁=Q, P, S or H; X₂₂=T, S or W; X₂₃=P, I, R or L; X₂₄=Y, T, P or L; X₂₅=absent, T, I or G; X₂₆=absent, T or L; X₂₇=absent or T; X₂₈=S or E; X₂₉=Y or L; X₃₀=Y, G, A or S; X₃₁=M or I; X₃₂=H or S; X₃₃=I, G or V; X₃₄=I or F; X₃₅=N, S, I or D; X₃₆=P or Y; X₃₇=S, D, I or E; X₃₈=G, F or D; X₃₉=G or S; X₄₀=S, N, T or E; X₄₁=T, K or A; X₄₂=S, Y, N or I; X₄₃=Q or D; X₄₄=K or S; X₄₅=F or V; and X₄₆ is Q or K.

In some embodiments LC-CDR1 is one of RSSQSLLHSNGYNYLD (SEQ ID NO:12), RASQSVSSSFLA (SEQ ID NO:15), RASQSVSSSYLA (SEQ ID NO:18), RSSQSLLHSDGYNYFD (SEQ ID NO:20), RASQSVSSGYLA (SEQ ID NO:23) or TTSQSVSSTSLD (SEQ ID NO:26).

In some embodiments LC-CDR2 is one of LGSNRAS (SEQ ID NO:13), GASSRAT (SEQ ID NO:16), LGSNRAA (SEQ ID NO:21) or DASSRAT (SEQ ID NO:24).

In some embodiments LC-CDR3 is one of MQALQTPYT (SEQ ID NO:14), QQYGPSIT (SEQ ID NO:17), QQYGSSPPIT (SEQ ID NO:19), MQGTHWPPT (SEQ ID NO:22), QQYGSSRPGLT (SEQ ID NO:25) or QQYGSSLLT (SEQ ID NO:27).

In some embodiments in accordance with any aspect of the present invention, HC-CDR1 may be SYX₃₀X₃₁X₃₂ (SEQ ID NO: 58), X₂₈X₂₉X₃₀MH (SEQ ID NO: 59) or SYX₃₀MH (SEQ ID NO: 60), wherein X₂₈=S or E; X₂₉=Y or L; X₃₀=Y, G, A or S; X₃₁=M or I; and X₃₂=H or S.

In some embodiments HC-CDR1 is one of SYYMH (SEQ ID NO:28), SYGMH (SEQ ID NO:31), SYAMH (SEQ ID NO:34), SYAIS (SEQ ID NO:36), or ELSMH (SEQ ID NO:39).

In some embodiments HC-CDR2 is one of IINPSGGSTSYAQKFQG (SEQ ID NO:29) VISYDGSNKYYADSVKG (SEQ ID NO:32), GIIPIFGTANYAQKFQG (SEQ ID NO:37) or GFDPEDGETIYAQKFQG (SEQ ID NO:40).

In some embodiments the antibody, or antigen binding fragment, may comprise at least one light chain variable region incorporating the following CDRs:

(SEQ ID NO: 53)

LC-CDR1: X₁X₂SQSX₃X₄X₅X₆X₇X₈X₉X₁₀X₁₁X₁₂X₁₃

(SEQ ID NO: 54)

LC-CDR2: X₁₄X₁₅SX₁₆RAX₁₇

(SEQ ID NO: 55)

LC-CDR3: X₁₈QX₁₉X₂₀X₂₁X₂₂X₂₃X₂₄X₂₅X₂₆X₂₇;

where X₁=R or T; X₂=S, A or T; X₃=L or V; X₄=L or S; X₅=H or S; X₆=S, G or T; X₇=N, F, Y, D or S; X₈=G or L; X₉=Y, A or D; X₁₀=absent or N; X₁₁=absent or Y; X₁₂=absent, L or F; X₁₃=absent (i.e. no amino acid) or D; X₁₄=L, G or D; X₁₅=G or A; X₁₆=N or S; X₁₇=S, T or A; X₁₈=M or Q; X₁₉=A, Y or G; X₂₀=L, G or T; X₂₁=Q, P, S or H; X₂₂=T, S or W; X₂₃=P, I, R or L; X₂₄=Y, T, P or L; X₂₅=absent, T, I or G; X₂₆=absent, T or L; and X₂₇=absent or T.

In some embodiments the antibody, or antigen binding fragment, may comprise at least one light chain variable region incorporating the following CDRs:

(SEQ ID NO: 12)

LC-CDR1: RSSQSLLHSNGYNYLD

(SEQ ID NO: 13)

LC-CDR2: LGSNRAS

(SEQ ID NO: 14)

LC-CDR3: MQALQTPYT

In some embodiments the antibody, or antigen binding fragment, may comprise at least one light chain variable region incorporating the following CDRs:

(SEQ ID NO: 15)

LC-CDR1: RASQSVSSSFLA

(SEQ ID NO: 16)

LC-CDR2: GASSRAT

(SEQ ID NO: 17)

LC-CDR3: QQYGPSIT

In some embodiments the antibody, or antigen binding fragment, may comprise at least one light chain variable region incorporating the following CDRs:

(SEQ ID NO: 18)

LC-CDR1: RASQSVSSSYLA

(SEQ ID NO: 16)

LC-CDR2: GASSRAT

(SEQ ID NO: 19)

LC-CDR3: QQYGSSPPIT

In some embodiments the antibody, or antigen binding fragment, may comprise at least one light chain variable region incorporating the following CDRs:

(SEQ ID NO: 20)

LC-CDR1: RSSQSLLHSDGYNYFD

(SEQ ID NO: 21)

LC-CDR2: LGSNRAA

(SEQ ID NO: 22)

LC-CDR3: MQGTHWPPT

In some embodiments the antibody, or antigen binding fragment, may comprise at least one light chain variable region incorporating the following CDRs:

(SEQ ID NO: 23)

LC-CDR1: RASQSVSSGYLA

(SEQ ID NO: 24)

LC-CDR2: DASSRAT

(SEQ ID NO: 25)

LC-CDR3: QQYGSSRPGLT

In some embodiments the antibody, or antigen binding fragment, may comprise at least one light chain variable region incorporating the following CDRs:

(SEQ ID NO: 26)

LC-CDR1: TTSQSVSSTSLD

(SEQ ID NO: 16)

LC-CDR2: GASSRAT

(SEQ ID NO: 27)

LC-CDR3: QQYGSSLLT

In some embodiments the antibody, or antigen binding fragment, may comprise at least one heavy chain variable region incorporating the following CDRs:

HC-CDR1:

(SEQ ID NO: 56)

X₂₈X₂₉X₃₀X₃₁X₃₂;
HC-CDR2:

(SEQ ID NO: 57)

X₃₃X₃₄X₃₅X₃₆X₃₇X₃₈X₃₉X₄₀X₄₁X₄₂YAX₄₃X₄₄X₄₅X₄₆G;
HC-CDR3:

(SEQ ID NO: 30)

one of PFGDFDY,

(SEQ ID NO: 33)

LPGWGAYAFDI,

(SEQ ID NO: 35)

DPDAANWGFLLYYGMDV,

(SEQ ID NO: 38)

ALADFWSGYYYYYYMDV,
or

(SEQ ID NO: 41)

TWFGELYY;

where X₂₈=S or E; X₂₃=Y or L; X₃₀=Y, G, A or S; X₃₁=M or I; X₃₂=H or S; X₃₃=I, G or V; X₃₄=I or F; X₃₅=N, S, I or D; X₃₆=P or Y; X₃₇=S, D, I or E; X₃₈=G, F or D; X₃₃=G or S; X₄₀=S, N, T or E; X₄₁=T, K or A; X₄₂=S, Y, N or I; X₄₃=Q or D; X₄₄=K or S; X₄₅=F or V; and X₄₆ is Q or K.

In some embodiments the antibody, or antigen binding fragment, may comprise at least one heavy chain variable region incorporating the following CDRs:

(SEQ ID NO: 28)

HC-CDR1: SYYMH

(SEQ ID NO: 29)

HC-CDR2: IINPSGGSTSYAQKFQG

(SEQ ID NO: 30)

HC-CDR3: PFGDFDY

In some embodiments the antibody, or antigen binding fragment, may comprise at least one heavy chain variable region incorporating the following CDRs:

(SEQ ID NO: 31)

HC-CDR1: SYGMH

(SEQ ID NO: 32)

HC-CDR2: VISYDGSNKYYADSVKG

(SEQ ID NO: 33)

HC-CDR3: LPGWGAYAFDI

In some embodiments the antibody, or antigen binding fragment, may comprise at least one heavy chain variable region incorporating the following CDRs:

(SEQ ID NO: 34)

HC-CDR1: SYAMH

(SEQ ID NO: 32)

HC-CDR2: VISYDGSNKYYADSVKG

(SEQ ID NO: 35)

HC-CDR3: DPDAANWGFLLYYGMDV

In some embodiments the antibody, or antigen binding fragment, may comprise at least one heavy chain variable region incorporating the following CDRs:

(SEQ ID NO: 36)

HC-CDR1: SYAIS

(SEQ ID NO: 37)

HC-CDR2: GIIPIFGTANYAQKFQG

(SEQ ID NO: 38)

HC-CDR3: ALADFWSGYYYYYYMDV

In some embodiments the antibody, or antigen binding fragment, may comprise at least one heavy chain variable region incorporating the following CDRs:

(SEQ ID NO: 39)

HC-CDR1: ELSMH

(SEQ ID NO: 40)

HC-CDR2: GFDPEDGETIYAQKFQG

(SEQ ID NO: 41)

HC-CDR3: TWFGELYY

The antibody may comprise at least one light chain variable region incorporating the CDRs shown in FIG. 1 or 3. The antibody may comprise at least one heavy chain variable region incorporating the CDRs shown in FIG. 2 or 3.

The antibody may comprise at least one light chain variable region (V_{L}) comprising the amino acid sequence of one of SEQ ID NOs 1, 12, 13, 14; or 2, 15, 16, 17; or 3, 18, 16, 19; or 4, 20, 21, 22; or 5, 23, 24, 25; or 6, 26, 16, 27, or one of the amino acid sequences shown in FIG. 1 or an amino acid sequence having at least 70%, more preferably one of at least 75%, 80%, 85%, 86%, 87%, 88%, 89%, 90%, 91%, 92%, 93%, 94%, 95%, 96%, 97%, 98%, 99%, or 100%, sequence identity to one of SEQ ID NOs SEQ ID NOs 1, 12, 13, 14; or 2, 15, 16, 17; or 3, 18, 16, 19; or 4, 20, 21, 22; or 5, 23, 24, 25; or 6, 26, 16, 27, or to the amino acid sequence of the V_{L} chain amino acid sequence shown in FIG. 1.

The antibody may comprise at least one heavy chain variable region (V_{H}) comprising the amino acid sequence of one of SEQ ID NOs 7, 28, 29, 30; or 8, 31, 32, 33; or 9, 34, 32, 35; or 10, 36, 37, 38; or 11, 39, 40, 41, or one of the amino acid sequences shown in FIG. 2 or an amino acid sequence having at least 70%, more preferably one of at least 75%, 80%, 85%, 86%, 87%, 88%, 89%, 90%, 91%, 92%, 93%, 94%, 95%, 96%, 97%, 98%, 99%, or 100%, sequence identity to one of SEQ ID NOs 7, 28, 29, 30; or 8, 31, 32, 33; or 9, 34, 32, 35; or 10, 36, 37, 38; or 11, 39, 40, 41, or to the amino acid sequence of the V_{H} chain amino acid sequence shown in FIG. 2.

The antibody may comprise at least one light chain variable region comprising the amino acid sequence of one of SEQ ID NOs 1, 12, 13, 14; or 2, 15, 16, 17; or 3, 18, 16, 19; or 4, 20, 21, 22; or 5, 23, 24, 25; or 6, 26, 16, 27, or one of the amino acid sequences shown in FIG. 1 (or an amino acid sequence having at least 70%, more preferably one of at least 75%, 80%, 85%, 90%, 95%, 96%, 97%, 98%, 99% or 100%, sequence identity to one of SEQ ID NOs 1, 12, 13, 14; or 2, 15, 16, 17; or 3, 18, 16, 19; or 4, 20, 21, 22; or 5, 23, 24, 25; or 6, 26, 16, 27, or to one of the amino acid sequences of the V_{L} chain amino acid sequence shown in FIG. 1) and at least one heavy chain variable region comprising the amino acid sequence of one of SEQ ID NOs 7, 28, 29, 30; or 8, 31, 32, 33; or 9, 34, 32, 35; or 10, 36, 37, 38; or 11, 39, 40, 41, or one of the amino acid sequence shown in FIG. 2 (or an amino acid sequence having at least 70%, more preferably one of at least 75%, 80%, 85%, 86%, 87%, 88%, 89%, 90%, 91%, 92%, 93%, 94%, 95%, 96%, 97%, 98%, 99%, or 100%, sequence identity to one of SEQ ID NOs 7, 28, 29, 30; or 8, 31, 32, 33; or 9, 34, 32, 35; or 10, 36, 37, 38; or 11, 39, 40, 41, or to one of the amino acid sequences of the V_{H} chain amino acid sequence shown in FIG. 2).

The antibody may optionally bind LAG-3, optionally human or murine LAG-3. The antibody may optionally have amino acid sequence components as described above. The antibody may be an IgG. In one embodiment an in vitro complex, optionally isolated, comprising an antibody, or antigen binding fragment, as described herein, bound to LAG-3 is provided.

The antibody may optionally inhibit or prevent interaction or functional association between human LAG-3 and human MHC class II, or between murine LAG-3 and murine MHC class II.

Such inhibition or prevention of interaction or functional association between LAG-3 and MHC class II may inhibit or prevent MHC class II-mediated activation of LAG-3 or MHC class II/LAG-3 signalling.

In one aspect of the present invention an isolated light chain variable region polypeptide is provided, the light chain variable region polypeptide comprising the following CDRs:

(SEQ ID NO: 53)

LC-CDR1: X₁X₂SQSX₃X₄X₅X₆X₇X₈X₉X₁₀X₁₁X₁₂X₁₃

(SEQ ID NO: 54)

LC-CDR2: X₁₄X₁₅SX₁₆RAX₁₇

(SEQ ID NO: 55)

LC-CDR3: X₁₈QX₁₉X₂₀X₂₁X₂₂X₂₃X₂₄X₂₅X₂₆X₂₇;

where X₁=R or T; X₂=S, A or T; X₃=L or V; X₄=L or S; X₅=H or S; X₆=S, G or T; X₇=N, F, Y, D or S; X₈=G or L; X₉=Y, A or D; X₁₀=absent or N; X₁₁=absent or Y; X₁₂=absent, L or F; X₁₃=absent (i.e. no amino acid) or D; X₁₄=L, G or D; X₁₅=G or A; X₁₆=N or S; X₁₇=S, T or A; X₁₈=M or Q; X₁₉=A, Y or G; X₂₀=L, G or T; X₂₁=Q, P, S or H; X₂₂=T, S or W; X₂₃=P, I, R or L; X₂₄=Y, T, P or L; X₂₅=absent, T, I or G; X₂₆=absent, T or L; and X₂₇=absent or T.

In some embodiments LC-CDR1 is one of RSSQSLLHSNGYNYLD (SEQ ID NO:12), RASQSVSSSFLA (SEQ ID NO:15), RASQSVSSSYLA (SEQ ID NO:18), RSSQSLLHSDGYNYFD (SEQ ID NO:20), RASQSVSSGYLA (SEQ ID NO:23) or TTSQSVSSTSLD (SEQ ID NO:26). In some embodiments LC-CDR2 is one of LGSNRAS (SEQ ID NO:13), GASSRAT (SEQ ID NO:16), LGSNRAA (SEQ ID NO:21) or DASSRAT (SEQ ID NO:24). In some embodiments LC-CDR3 is one of MQALQTPYT (SEQ ID NO:14), QQYGPSIT (SEQ ID NO:17), QQYGSSPPIT (SEQ ID NO:19), MQGTHWPPT (SEQ ID NO:22), QQYGSSRPGLT (SEQ ID NO:25) or QQYGSSLLT (SEQ ID NO:27). In some embodiments the isolated light chain variable region polypeptide is capable of binding to LAG-3.

In one aspect of the present invention an isolated light chain variable region polypeptide is provided, comprising an amino acid sequence having at least 85% sequence identity to the light chain sequence: SEQ ID NO:1, 2, 3, 4, 5 or 6 (FIG. 1). In some embodiments the isolated light chain variable region polypeptide is capable of binding to LAG-3.

In one aspect of the present invention an isolated heavy chain variable region polypeptide is provided, the heavy chain variable region polypeptide comprising the following CDRs:

HC-CDR1:

(SEQ ID NO: 56)

X₂₈X₂₉X₃₀X₃₁X₃₂;
HC-CDR2:

(SEQ ID NO: 57)

X₃₃X₃₄X₃₅X₃₆X₃₇X₃₈X₃₉X₄₀X₄₁X₄₂YAX₄₃X₄₄X₄₅X₄₆G;
HC-CDR3:

(SEQ ID NO: 30)

one of PFGDFDY,

(SEQ ID NO: 33)

LPGWGAYAFDI,

(SEQ ID NO: 35)

DPDAANWGFLLYYGMDV,

(SEQ ID NO: 38)

ALADFWSGYYYYYYMDV,
or

(SEQ ID NO: 41)

TWFGELYY;

where X₂₈=S or E; X₂₃=Y or L; X₃₀=Y, G, A or S; X₃₁=M or I; X₃₂=H or S; X₃₃=I, G or V; X₃₄=I or F; X₃₅=N, S, I or D; X₃₆=P or Y; X₃₇=S, D, I or E; X₃₈=G, F or D; X₃₉=G or S; X₄₀=S, N, T or E; X₄₁=T, K or A; X₄₂=S, Y, N or I; X₄₃=Q or D; X₄₄=K or S; X₄₅=F or V; and X₄₆ is Q or K.

In some embodiments, HC-CDR1 is one of SYYMH (SEQ ID NO:28), SYGMH (SEQ ID NO:31), SYAMH (SEQ ID NO:34), SYAIS (SEQ ID NO:36), or ELSMH (SEQ ID NO:39). In some embodiments HC-CDR2 is one of IINPSGGSTSYAQKFQG (SEQ ID NO:29) VISYDGSNKYYADSVKG (SEQ ID NO:32), GIIPIFGTANYAQKFQG (SEQ ID NO:37) or EGFDPEDGETIYAQKFQG (SEQ ID NO:40). In some embodiments the isolated heavy chain variable region polypeptide is capable of binding to LAG-3.

In one aspect of the present invention an isolated heavy chain variable region polypeptide is provided, comprising an amino acid sequence having at least 85% sequence identity to the heavy chain sequence of SEQ ID NO:7, 8, 9, 10 or 11 (FIG. 2). In some embodiments the isolated heavy chain variable region polypeptide is capable of binding to LAG-3.

In one aspect of the present invention an antibody, or antigen binding fragment, is provided, the antibody, or antigen binding fragment, comprising a heavy chain and a light chain variable region sequence, wherein:

the light chain comprises a LC-CDR1, LC-CDR2, LC-CDR3, having at least 85% overall sequence identity to LC-CDR1: one of X₁X₂SQSX₃X₄X₅X₆X₇X₈X₉X₁₀X₁₁X₁₂X₁₃ (SEQ ID NO:53), RSSQSLLHSNGYNYLD (SEQ ID NO:12), RASQSVSSSFLA (SEQ ID NO:15), RASQSVSSSYLA (SEQ ID NO:18), RSSQSLLHSDGYNYFD (SEQ ID NO:20), RASQSVSSGYLA (SEQ ID NO:23) or TTSQSVSSTSLD (SEQ ID NO:26), LC-CDR2: one of X₁₄X₁₅SX₁₆RAX₁₇ (SEQ ID NO:54), LGSNRAS (SEQ ID NO:13), GASSRAT (SEQ ID NO:16), LGSNRAA (SEQ ID NO:21) or DASSRAT (SEQ ID NO:24), LC-CDR3: one of X₁₈QX₁₉X₂₀X₂₁X₂₂X₂₃X₂₄X₂₅X₂₆X₂₇ (SEQ ID NO:55), MQALQTPYT (SEQ ID NO:14), QQYGPSIT (SEQ ID NO:17), QQYGSSPPIT (SEQ ID NO:19), MQGTHWPPT (SEQ ID NO:22), QQYGSSRPGLT (SEQ ID NO:25) or QQYGSSLLT (SEQ ID NO:27), respectively, where X₁=R or T; X₂=S, A or T; X₃=L or V; X₄=L or S; X₅=H or S; X₆=S, G or T; X₇=N, F, Y, D or S; X₈=G or L; X₉=Y, A or D; X₁₀=absent or N; X₁₁=absent or Y; X₁₂=absent, L or F; X₁₃=absent (i.e. no amino acid) or D; X₁₄=L, G or D; X₁₅=G or A; X₁₆=N or S; X₁₇=S, T or A; X₁₈=M or Q; X₁₉=A, Y or G; X₂₀=L, G or T; X₂₁=Q, P, S or H; X₂₂=T, S or W; X₂₃=P, I, R or L; X₂₄=Y, T, P, or L; X₂₅=absent, T I or G; X₂₆=absent, T or L; and X₂₇=absent or T, and;
the heavy chain comprises a HC-CDR1, HC-CDR2, HC-CDR3, having at least 85% overall sequence identity to HC-CDR1: one of X₂₈X₂₉X₃₀X₃₁X₃₂ (SEQ ID NO:56), SYYMH (SEQ ID NO:28), SYGMH (SEQ ID NO:31), SYAMH (SEQ ID NO:34), SYAIS (SEQ ID NO:36), or ELSMH (SEQ ID NO:39), HC-CDR2: one of X₃₃X₃₄X₃₅X₃₆X₃₇X₃₈X₃₉X₄₀X₄₁X₄₂YAX₄₃X₄₄X₄₅X₄₆G (SEQ ID NO:57), IINPSGGSTSYAQKFQG (SEQ ID NO:29) VISYDGSNKYYADSVKG (SEQ ID NO:32), GIIPIFGTANYAQKFQG (SEQ ID NO:37) or GFDPEDGETIYAQKFQG (SEQ ID NO:40), HC-CDR3: one of PFGDFDY (SEQ ID NO:30), LPGWGAYAFDI (SEQ ID NO:33), DPDAANWGFLLYYGMDV (SEQ ID NO:35), ALADFWSGYYYYYYMDV (SEQ ID NO:38), or TWFGELYY (SEQ ID NO:41), respectively, where X₂₈=S or E; X₂₉=Y or L; X₃₀=Y, G, A or S; X₃₁=M or I; X₃₂=H or S; X₃₃=I, G or V; X₃₄=I or F; X₃₅=N, S, I or D; X₃₆=P or Y; X₃₇=S, D, I or E; X₃₈=G, F or D; X₃₉=G or S; X₄₀=S, N, T or E; X₄₁=T, K or A; X₄₂=S, Y, N or I; X₄₃=Q or D; X₄₄=K or S; X₄₅=F or V; and X₄₆ is Q or K.

In some embodiments the degree of sequence identity may be one of 86%, 87%, 88%, 89%, 90%, 91%, 92%, 93%, 94%, 95%, 96%, 97%, 98%, 99%, or 100%.

In another aspect of the present invention an antibody, or antigen binding fragment, optionally isolated, is provided comprising a heavy chain and a light chain variable region sequence, wherein:

the light chain sequence has at least 85% sequence identity to the light chain sequence: SEQ ID NO:1, 2, 3, 4, 5 or 6 (FIG. 1), and;
the heavy chain sequence has at least 85% sequence identity to the heavy chain sequence of SEQ ID NO:7, 8, 9, 10 or 11 (FIG. 2).

In some embodiments the degree of sequence identity may be one of 86%, 87%, 88%, 89%, 90%, 91%, 92%, 93%, 94%, 95%, 96%, 97%, 98%, 99%, or 100%.

In some embodiments the antibody, antigen binding fragment, or polypeptide further comprises variable region light chain framework sequences between the CDRs according to the arrangement LCFR1:LC-CDR1:LCFR2:LC-CDR2:LCFR3:LC-CDR3:LCFR4. The framework sequences may be derived from human consensus framework sequences.

In one aspect of the present invention an isolated light chain variable region polypeptide, optionally in combination with a heavy chain variable region polypeptide as described herein, is provided, the light chain variable region polypeptide comprising the following CDRs:

(SEQ ID NO: 53)

LC-CDR1: X₁X₂SQSX₃X₄X₅X₆X₇X₈X₉X₁₀X₁₁X₁₂X₁₃

(SEQ ID NO: 54)

LC-CDR2: X₁₄X₁₅SX₁₆RAX₁₇

(SEQ ID NO: 55)

LC-CDR3: X₁₈QX₁₉X₂₀X₂₁X₂₂X₂₃X₂₄X₂₅X₂₆X₂₇;

where X₁=R or T; X₂=S, A or T; X₃=L or V; X₄=L or S; X₅=H or S; X₆=S, G or T; X₇=N, F, Y, D or S; X₈=G or L; X₉=Y, A or D; X₁₀=absent or N; X₁₁=absent or Y; X₁₂=absent, L or F; X₁₃=absent (i.e. no amino acid) or D; X₁₄=L, G or D; X₁₅=G or A; X₁₆=N or S; X₁₇=S, T or A; X₁₈=M or Q; X₁₉=A, Y or G; X₂₀=L, G or T; X₂₁=Q, P, S or H; X₂₂=T, S or W; X₂₃=P, I, R or L; X₂₄=Y, T, P or L; X₂₅=absent, T, I or G; X₂₆=absent, T or L; and X₂₇=absent or T.

In some embodiments LC-CDR1 is one of RSSQSLLHSNGYNYLD (SEQ ID NO:12), RASQSVSSSFLA (SEQ ID NO:15), RASQSVSSSYLA (SEQ ID NO:18), RSSQSLLHSDGYNYFD (SEQ ID NO:20), RASQSVSSGYLA (SEQ ID NO:23) or TTSQSVSSTSLD (SEQ ID NO:26). In some embodiments LC-CDR2 is one of LGSNRAS (SEQ ID NO:13), GASSRAT (SEQ ID NO:16), LGSNRAA (SEQ ID NO:21) or DASSRAT (SEQ ID NO:24). In some embodiments LC-CDR3 is one of MQALQTPYT (SEQ ID NO:14), QQYGPSIT (SEQ ID NO:17), QQYGSSPPIT (SEQ ID NO:19), MQGTHWPPT (SEQ ID NO:22), QQYGSSRPGLT (SEQ ID NO:25) or QQYGSSLLT (SEQ ID NO:27).

In some embodiments the antibody, antigen binding fragment, or polypeptide further comprises variable region heavy chain framework sequences between the CDRs according to the arrangement HCFR1:HC-CDR1:HCFR2:HC-CDR2:HCFR3:HC-CDR3:HCFR4. The framework sequences may be derived from human consensus framework sequences.

In one aspect of the present invention an isolated heavy chain variable region polypeptide, optionally in combination with a light chain variable region polypeptide as described herein, is provided, the heavy chain variable region polypeptide comprising the following CDRs:

HC-CDR1:

(SEQ ID NO: 56)

X₂₈X₂₉X₃₀X₃₁X_{32;}
HC-CDR2:

(SEQ ID NO: 57)

X₃₃X₃₄X₃₅X₃₆X₃₇X₃₈X₃₉X₄₀X₄₁X₄₂YAX₄₃X₄₄X₄₅X₄₆G;
HC-CDR3:

(SEQ ID NO: 30)

one of PFGDFDY,

(SEQ ID NO: 33)

LPGWGAYAFDI,

(SEQ ID NO: 35)

DPDAANWGFLLYYGMDV,

(SEQ ID NO: 38)

ALADFWSGYYYYYYMDV,
or

(SEQ ID NO: 41)

TWFGELYY;

where X₂₈=S or E; X₂₉=Y or L; X₃₀=Y, G, A or S; X₃₁=M or I; X₃₂=H or S; X₃₃=I, G or V; X₃₄=I or F; X₃₅=N, S, I or D; X₃₆=P or Y; X₃₇=S, D, I or E; X₃₈=G, F or D; X₃₉=G or S; X₄₀=S, N, T or E; X₄₁=T, K or A; X₄₂=S, Y, N or I; X₄₃=Q or D; X₄₄=K or S; X₄₅=F or V; and X₄₆ is Q or K.

In some embodiments HC-CDR1 is one of SYYMH (SEQ ID NO:28), SYGMH (SEQ ID NO:31), SYAMH (SEQ ID NO:34), SYAIS (SEQ ID NO:36), or ELSMH (SEQ ID NO:39).

In some embodiments HC-CDR2 is one of IINPSGGSTSYAQKFQG (SEQ ID NO:29) VISYDGSNKYYADSVKG (SEQ ID NO:32), GIIPIFGTANYAQKFQG (SEQ ID NO:37) or GFDPEDGETIYAQKFQG (SEQ ID NO:40).

In some embodiments, the antibody, or antibody binding fragment, may further comprise a human constant region. For example selected from one of IgG1, IgG2, IgG3 and IgG4.

In some embodiments, the antibody, or antibody binding fragment, may further comprise a murine constant region. For example, selected from one of IgG1, IgG2A, IgG2B and IgG3.

In another aspect of the present invention, an antibody or antigen binding fragment, optionally isolated, which is capable of binding to LAG-3, which is a bispecific antibody or a bispecific antigen binding fragment is provided. The bispecific antibody or antigen binding fragment comprises (i) an antigen binding fragment or polypeptide capable of binding to LAG-3 as described herein, and (ii) an antigen binding fragment or polypeptide which is capable of binding to a target protein other than LAG-3.

In some embodiments, the target protein other than LAG-3 may be a cell surface receptor, e.g. a receptor expressed on the cell surface of T cells. In some embodiments the cell surface receptor may be an immune checkpoint receptor, e.g. a costimulatory receptor or an inhibitory receptor. In some embodiments, the costimulatory receptor may be selected from CD27, CD28, ICOS, CD40, CD122, OX43, 4-1BB and GITR. In some embodiments, the inhibitory receptor may be selected from B7-H3, B7-H4, BTLA, CTLA-4, A2AR, VISTA, TIM-3, PD-1, and KIR.

In some embodiments, the target protein other than LAG-3 may be a cancer marker whose expression is associated with a cancer. In some embodiments, the cancer marker may be expressed at the cell surface. In some embodiments, cancer marker may be selected from HER-2, HER-3, EGFR, EpCAM, CD30, CD33, CD38, CD20, CD24, CD90, CD15, CD52, CA-125, CD34, CA-15-3, CA-19-9, CEA, CD99, CD117, CD31, CD44, CD123, CD133, ABCB5 and CD45.

In another aspect of the present invention a chimeric antigen receptor (CAR) is provided, comprising an antigen binding fragment as described herein.

In another aspect the present invention provides a cell comprising a CAR as described herein.

In another aspect of the present invention an in vitro complex is provided, comprising an antibody, antigen binding fragment, polypeptide, CAR or cell as described herein bound to LAG-3. The in vitro complex may optionally be isolated.

In another aspect of the present invention, a composition, e.g. a pharmaceutical composition or medicament, is provided. The composition may comprise an antibody, antigen binding fragment, polypeptide, CAR or cell as described herein and at least one pharmaceutically-acceptable carrier, excipient, adjuvant or diluent.

In another aspect of the present invention an isolated nucleic acid encoding an antibody, antigen binding fragment, polypeptide, or CAR as described herein is provided. The nucleic acid may have a sequence of one of SEQ ID NOs 42, 43, 44, 45, 46, 47, 48, 49, 50, 51, or 52 (FIG. 4), or a coding sequence which is degenerate as a result of the genetic code, or may have a nucleotide sequence having at least 70% identity thereto, optionally one of 75%, 80%, 85%, 86%, 87%, 88%, 89%, 90%, 91%, 92%, 93%, 94%, 95%, 96%, 97%, 98%, 99%, or 100%.

In one aspect of the present invention there is provided a vector comprising a nucleic acid described herein. In another aspect of the present invention, there is provided a host cell comprising the vector. For example, the host cell may be eukaryotic, or mammalian, e.g. Chinese Hamster Ovary (CHO), or human or may be a prokaryotic cell, e.g. E. coll. In one aspect of the present invention a method for making an antibody, antigen binding fragment, polypeptide or CAR as described herein is provided, the method comprising culturing a host cell as described herein under conditions suitable for the expression of a vector encoding the antibody, antigen binding fragment, polypeptide or CAR, and recovering the antibody, antigen binding fragment, polypeptide or CAR.

In another aspect of the present invention an antibody, antigen binding fragment, polypeptide, CAR, cell or composition is provided for use in therapy, or in a method of medical treatment. In another aspect of the present invention an antibody, antigen binding fragment, polypeptide, CAR, cell or composition as described herein is provided for use in the treatment of a T-cell dysfunctional disorder. In another aspect of the present invention, the use of an antibody, antigen binding fragment, polypeptide, CAR, cell or composition as described herein in the manufacture of a medicament or pharmaceutical composition for use in the treatment of a T-cell dysfunctional disorder is provided.

In another aspect of the present invention a method of enhancing T-cell function comprising administering an antibody, antigen binding fragment, polypeptide, CAR, cell or composition as described herein to a dysfunctional T-cell is provided. The method may be performed in vitro or in vivo.

In another aspect of the present invention a method of treating a T-cell dysfunctional disorder is provided, the method comprising administering an antibody, antigen binding fragment, polypeptide, CAR, cell or composition as described herein to a patient suffering from a T-cell dysfunctional disorder.

In another aspect of the present invention an antibody, antigen binding fragment, polypeptide, CAR, cell or composition is provided for use in the treatment of a cancer. In another aspect of the present invention, the use of an antibody, antigen binding fragment, polypeptide, CAR, cell or composition as described herein in the manufacture of a medicament or pharmaceutical composition for use in the treatment of a cancer is provided.

In another aspect of the present invention a method of killing a tumour cell is provided, the method comprising administering an antibody, antigen binding fragment, polypeptide, CAR, cell or composition as described herein to a tumour cell. The method may be performed in vitro or in vivo. Killing of a tumour cell may, for example, be as a result of antibody dependent cell-mediated cytotoxicity (ADCC), complement dependent cytotoxicity (CDC), or through the action of a drug conjugated to the antibody, antigen binding fragment, polypeptide, CAR, cell or composition.

In another aspect of the present invention a method of treating a cancer is provided, the method comprising administering an antibody, antigen binding fragment, polypeptide, CAR, cell or composition as described herein to a patient suffering from a cancer.

The cancer may be a cancer which overexpresses LAG-3, or may comprise cells which overexpress LAG-3.

In another aspect of the present invention a method of modulating an immune response in a subject is provided, the method comprising administering to the subject an antibody, antigen binding fragment, polypeptide, CAR, cell or composition as described herein such that the immune response in the subject is modulated.

In another aspect of the present invention a method of inhibiting growth of tumor cells is provided, comprising administering an antibody, antigen binding fragment, polypeptide, CAR, cell or composition as described herein. The method may be in vitro or in vivo. In some embodiments a method of inhibiting growth of tumor cells in a subject is provided, the method comprising administering to the subject a therapeutically effective amount of an antibody, antigen binding fragment, polypeptide, CAR, cell or composition as described herein.

In another aspect of the present invention a method is provided, the method comprising contacting a sample containing, or suspected to contain, LAG-3 with an antibody, antigen binding fragment, CAR or cell as described herein, and detecting the formation of a complex of antibody, antigen binding fragment, CAR or cell and LAG-3.

In another aspect of the present invention a method of diagnosing a disease or condition in a subject is provided, the method comprising contacting, in vitro, a sample from the subject with an antibody, antigen binding fragment, CAR or cell as described herein, and detecting the formation of a complex of antibody, or antigen binding fragment, CAR or cell and LAG-3.

In a further aspect of the present invention the use of an antibody, antigen binding fragment, CAR or cell as described herein, for the detection of LAG-3 in vitro is provided. In another aspect of the present invention the use of an antibody, antigen binding fragment, CAR or cell as described herein, as an in vitro diagnostic agent is provided.

In methods of the present invention the antibody, antigen binding fragment, polypeptide, CAR or cell may be provided as a composition as described herein.

In another aspect the present invention provides a method of treating or preventing a cancer in a subject, comprising:
(a) isolating at least one cell from a subject;
(b) modifying the at least one cell to express or comprise the antibody, antigen binding fragment, polypeptide, CAR, nucleic acid or vector described herein, and;
(c) administering the modified at least one cell to a subject.

In another aspect the present invention provides a method of treating or preventing a cancer in a subject, comprising:
(a) isolating at least one cell from a subject;
(b) introducing into the at least one cell the nucleic acid or vector described herein, thereby modifying the at least one cell, and;
(c) administering the modified at least one cell to a subject.

In another aspect the present invention provides a kit of parts comprising a predetermined quantity of the antibody, antigen binding fragment, polypeptide, CAR, composition, nucleic acid, vector or cell described herein.

In some embodiments the antibody may be clone A6, 1G11, C2, C12, F5 or G8 as described herein.

### DESCRIPTION

Antibodies

Antibodies according to the present invention preferably bind to LAG-3 (the antigen), preferably human or murine LAG-3, optionally with a K_{D} in the range 0.1 to 3 nM.

Antibodies according to the present invention may be provided in isolated form.

Antibodies according to the present invention may exhibit least one of the following properties:
a) binds to human, mouse or rhesus macaque LAG-3 with a K_{D} of 1 μM or less, preferably one of ≤10 nM, ≤5 nM, ≤3 nM, ≤2 nM, ≤1.5 nM, ≤1.4 nM, ≤1.3 nM, ≤1.25 nM, ≤1.24 nM, ≤1.23 nM, ≤1.22 nM, ≤1.21 nM, ≤1.2 nM, ≤1.15 nM, ≤1.1, nM ≤1.05 nM, ≤1 nM, ≤900 pM, ≤800 pM, ≤700 pM, ≤600 pM, ≤500 pM;
b) binds to human, mouse or rhesus macaque LAG-3 with a similar affinity to, or with greater affinity than, affinity of binding to human, mouse or rhesus macaque LAG-3 by BMS-986016;
c) binds to activated CD4+ T cells;
d) displays substantially no binding to unactivated CD4+ T cells;
e) inhibits or prevents interaction between LAG-3 and MHC class II, optionally human LAG-3 and human MHC class II (e.g. as determined analysis of inhibition of LAG-3 binding to Daudi cells);
f) inhibits or prevents interaction between LAG-3 and MHC class II, optionally human LAG-3 and human MHC class II, with an IC₅₀ of 1 μM or less, preferably one of ≤500 nM, ≤250 nM ≤200 nM, ≤150 nM, ≤120 nM, ≤110 nM, ≤100 nM, ≤90 nM, ≤80 nM, ≤70 nM, ≤60 nM, ≤50 nM, ≤30 nM, ≤30 nM, ≤20 nM, ≤10 nM, ≤5 nM, ≤2.5 nM, ≤2 nM, 1 nM;
g) inhibits or prevents interaction between LAG-3 and MHC class II, optionally human LAG-3 and human MHC class II, to a similar extent to, or to a greater extent than, inhibition/prevention of binding between LAG-3 and MHC class II by BMS-986016;
h) increases one or more of T-cell proliferation, IL-2 production and IFNγ production in an Mixed Lymphocyte Reaction (MLR) assay (e.g. see Bromelow et al J. Immunol Methods, 2001 Jan. 1; 247(1-2):1-8);
i) increases one or more of T-cell proliferation, IL-2 production and IFNγ production in an Mixed Lymphocyte Reaction (MLR) assay to a similar extent to, or to a greater extent than, BMS-986016;
j) binds to an epitope of LAG-3, optionally human LAG-3 which is different to the epitope of LAG-3 to which BMS-986016 binds;
k) increases one or more of T-cell proliferation, IL-2 production and IFNγ production in response to infection;
l) inhibits tumour growth, optionally in vivo.

In some embodiments, the antibody according to the present invention may be useful in methods for expanding a population of immune cells, e.g. T cells. The antibodies according to the invention are useful for expanding populations of immune cells with desirable properties.

In some embodiments, a population of immune cells expanded in the presence of an antibody according to the present invention (e.g. expanded from a population of PBMCs, e.g. by stimulation through the TCR, e.g. in the presence of IL-2) may possess one or more of the following properties as compared to a population of immune cells expanded by a comparable method, but in the absence of the antibody:
(i) a comparable total number of expanded cells;
(ii) a comparable number of T cells;
(iii) a lower ratio of CD8:CD4 cells (indicative of preferential expansion of CD4+ T cells over CD8+ T cells);
(iv) a lower proportion of Tregs (e.g. CD4+CD25+FoxP3+Tregs) within the T cell population (e.g. within the CD4+ T cell population);
(v) an higher proportion of T helper (Th) cells within the T cell population (e.g. within the CD4+ T cell population);
(vi) a lower proportion of PD1+ cells (e.g. CD8+ PD1+ T cells and/or CD4+PD1+ T cells) within the T cell population;
(vii) a comparable proportion of CTLA4+ cells (e.g. CD8+CTLA4+ T cells and/or CD4+CTLA4+ T cells) within the T cell population;
(viii) a comparable proportion of IL-13+ cells (e.g. CD8+IL-13+ T cells and/or CD4+IL-13+ T cells) within the T cell population;
(ix) a comparable proportion of IFNγ+ cells (e.g. CD8+IFNγ+ T cells and/or CD4+IFNγ+ T cells) within the T cell population;
(x) a comparable proportion of TNFα+ cells (e.g. CD8+TNFα+ T cells and/or CD4+TNFα+ T cells) within the T cell population;
(xi) a lower proportion of NK cells; and
(xii) a higher proportion of B cells.

In some embodiments, a population of immune cells expanded in the presence of an antibody according to the present invention (e.g. expanded from a population of PBMCs, e.g. by stimulation through the TCR, e.g. in the presence of IL-2) may possess one or more of the following properties as compared to a population of immune cells expanded by a comparable method, but in the absence of the antibody: a lower ratio of CD8:CD4 cells; a lower proportion of Tregs (e.g. CD4+CD25+FoxP3+ Tregs) within the CD4+ T cell population; a higher proportion of T helper (Th) cells within the T cell population; and a lower proportion of PD1+ cells within the T cell population.

By “antibody” we include a fragment or derivative thereof, or a synthetic antibody or synthetic antibody fragment.

In view of today's techniques in relation to monoclonal antibody technology, antibodies can be prepared to most antigens. The antigen-binding portion may be a part of an antibody (for example a Fab fragment) or a synthetic antibody fragment (for example a single chain Fv fragment [ScFv]). Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimeric antibodies are discussed by Neuberger et al (1988, 8th International Biotechnology Symposium Part 2, 792-799).

Monoclonal antibodies (mAbs) are useful in the methods of the invention and are a homogenous population of antibodies specifically targeting a single epitope on an antigen.

Polyclonal antibodies are useful in the methods of the invention. Monospecific polyclonal antibodies are preferred. Suitable polyclonal antibodies can be prepared using methods well known in the art.

Antigen binding fragments of antibodies, such as Fab and Fab₂ fragments may also be used/provided as can genetically engineered antibodies and antibody fragments. The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parent antibody (Morrison et al (1984) Proc. Natl. Acad. Sd. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sd. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

By “ScFv molecules” we mean molecules wherein the V_{H} and V_{L} partner domains are covalently linked, e.g. by a flexible oligopeptide. Fab, Fv, ScFv and dAb antibody fragments can all be expressed in and secreted from E. coli, thus allowing the facile production of large amounts of the said fragments.

Whole antibodies, and F(ab′)₂ fragments are “bivalent”. By “bivalent” we mean that the said antibodies and F(ab′)₂ fragments have two antigen combining sites. In contrast, Fab, Fv, ScFv and dAb fragments are monovalent, having only one antigen combining site. Synthetic antibodies which bind to LAG-3 may also be made using phage display technology as is well known in the art.

The present application also provides an antibody or antigen binding fragment which is capable of binding to LAG-3, and which is a bispecific antibody or a bispecific antigen binding fragment. In some embodiments, the bispecific antibody or bispecific antigen binding fragment may be isolated.

In some embodiments, the bispecific antibodies and bispecific antigen binding fragments comprise an antigen binding fragment or a polypeptide according to the present invention. In some embodiments, the bispecific antibodies and bispecific antigen binding fragments comprise an antigen binding fragment capable of binding to LAG-3, wherein the antigen binding fragment which is capable of binding to LAG-3 comprises or consists of an antigen binding fragment or a polypeptide according to the present invention.

In some embodiments the bispecific antibodies and bispecific antigen binding fragments comprise an antigen binding fragment capable of binding to LAG-3, and an antigen binding fragment capable of binding to another target protein.

The antigen binding fragment capable of binding to another target protein may be capable of binding to another protein other than LAG-3.

In some embodiments, the target protein may be a cell surface receptor. In some embodiments, the target protein may be a cell surface receptor expressed on the cell surface of an immune cell, e.g. T cell. In some embodiments the cell surface receptor may be an immune checkpoint receptor. In some embodiments, the immune checkpoint receptor may be a costimulatory receptor. In some embodiments, the costimulatory receptor may be selected from CD27, CD28, ICOS, CD40, CD122, OX43, 4-1BB and GITR. In some embodiments, the immune checkpoint receptor may be an inhibitory receptor. In some embodiments, the inhibitory receptor may be selected from B7-H3, B7-H4, BTLA, CTLA-4, A2AR, VISTA, TIM-3, PD-1, and KIR.

In some embodiments, the target protein may be a cancer marker. That is, the target protein may be a protein whose expression (e.g. upregulated expression) is associated with a cancer. In some embodiments, the cancer marker may be expressed at the cell surface. In some embodiments the cancer marker may be a receptor. In some embodiments, the cancer marker may be selected from HER-2, HER-3, EGFR, EpCAM, CD30, CD33, CD38, CD20, CD24, CD90, CD15, CD52, CA-125, CD34, CA-15-3, CA-19-9, CEA, CD99, CD117, CD31, CD44, CD123, CD133, ABCB5 and CD45.

In some embodiments, the antigen binding fragment for CD27 may comprise the CDRs, light and heavy chain variable domains or other CD27 binding fragment of e.g. anti-CD27 antibody clone 0323 (Millipore) or varlilumab (Celldex Therapeutics). In some embodiments, the antigen binding fragment for CD28 may comprise the CDRs, light and heavy chain variable domains or other CD28 binding fragment of e.g. anti-CD28 antibody clone CD28.6 (eBioscience), clone CD28.2, clone JJ319 (Novus Biologicals), clone 204.12, clone B-23, clone 10F3 (Thermo Scientific Pierce Antibodies), clone 37407 (R&D Systems), clone 204-12 (Abnova Corporation), clone 15E8 (EMD Millipore), clone 204-12, clone YTH913.12 (AbD Serotec), clone B-T3 (Acris Antibodies), clone 9H6E2 (Sino Biological), clone C28/77 (MyBioSource.com), clone KOLT-2 (ALPCO), clone 152-2E10 (Santa Cruz Biotechnology), or clone XPH-56 (Creative Diagnostics). In some embodiments, the antigen binding fragment for ICOS may comprise the CDRs, light and heavy chain variable domains or other ICOS binding fragment of e.g. anti-ICOS antibody clone ISA-3 (eBioscience), clone SP98 (Novus Biologicals), clone 1G1, clone 3G4 (Abnova Corporation), clone 669222 (R&D Systems), clone TQ09 (Creative Diagnostics), or clone C398.4A (BioLegend). In some embodiments, the antigen binding fragment for CD40 may comprise the CDRs, light and heavy chain variable domains or other CD40 binding fragment of e.g. anti-CD40 antibody clone 82111 (R&D Systems), or ASKP1240 (Okimura et al., AM J Transplant (2014) 14(6) 1290-1299). In some embodiments, the antigen binding fragment for CD122 may comprise the CDRs, light and heavy chain variable domains or other CD122 binding fragment of anti-CD122 antibody clone mikβ2 (PharMingen). In some embodiments, the antigen binding fragment for OX43 may comprise the CDRs, light and heavy chain variable domains or other OX43 binding fragment of e.g. anti-OX43 antibodies disclosed in US 20130280275, U.S. Pat. No. 8,283,450 or WO2013038191, e.g. clone 12H3 or clone 20E5. In some embodiments, the antigen binding fragment for 4-1BB may comprise the CDRs, light and heavy chain variable domains or other 4-1BB binding fragment of e.g. anti-4-1BB antibody PF-05082566 (Fisher et al., Cancer Immunol Immunother (2012) 61: 1721-1733), or urelumab (BMS-665513; Bristol-Myers Squibb; Li and Liu, Clin Pharmacol (2013); 5: 47-53). In some embodiments, the antigen binding fragment for GITR may comprise the CDRs, light and heavy chain variable domains or other GITR binding fragment of e.g. anti-GITR antibody TRX-518 (Tolerx®; Schaer et al., (2010) 11(12): 1378-1386), or clone AIT 518D (LifeSpan Biosciences). In some embodiments, the antigen binding fragment for B7-H3 may comprise the CDRs, light and heavy chain variable domains or other B7-H3 binding fragment of e.g. anti-B7-H3 antibody clones disclosed in US 20130078234, WO2014160627 or WO2011109400. In some embodiments, the antigen binding fragment for B7-H4 may comprise the CDRs, light and heavy chain variable domains or other B7-H4 binding fragment of e.g. anti-B7-H4 antibody clones disclosed in WO2013067492, WO2009073533 or EP2934575, for example clone 2H9. In some embodiments, the antigen binding fragment for BTLA may comprise the CDRs, light and heavy chain variable domains or other BTLA binding fragment of e.g. anti-BTLA antibody clone 167, clone 2G8, clone 4C5 (Abnova Corporation), clone 4B8 (antibodies-online), clone MIH26 (Thermo Scientific Pierce Antibodies), clone UMAB61 (OriGene Technologies), clone 330104 (R&D Systems), clone 1B4 (LifeSpan BioSciences), clone 440205, clone 5E7 (Creative Diagnostics). In some embodiments, the antigen binding fragment for CTLA4 may comprise the CDRs, light and heavy chain variable domains or other CTLA4 binding fragment of e.g. anti-CTLA4 antibody clone 2F1, clone 1F4 (Abnova Corporation), clone 9H10 (EMD Millipore), clone BNU3 (GeneTex), clone 1E2, clone AS32 (LifeSpan BioSciences) clone A3.4H2.H12 (Acris Antibodies), clone 060 (Sino Biological), clone BU5G3 (Creative Diagnostics), clone MIH8 (MBL International), clone A3.6B10.G1, or clone L3D10 (BioLegend). In some embodiments, the antigen binding fragment for A2AR may comprise the CDRs, light and heavy chain variable domains or other A2AR binding fragment of e.g. anti-A2AR antibody clone 7F6 (Millipore; Koshiba et al. Molecular Pharmacology (1999); 55: 614-624. In some embodiments, the antigen binding fragment for VISTA may comprise the CDRs, light and heavy chain variable domains or other VISTA binding fragment of e.g. anti-VISTA antibodies disclosed in WO2015097536 or US20140105912, e.g. clone 13F3. In some embodiments, the antigen binding fragment for TIM-3 may comprise the CDRs, light and heavy chain variable domains or other TIM-3 binding fragment of e.g. anti-TIM-3 antibody clone F38-2E2 (BioLegend), clone 2E2 (Merck Millipore; Pires da Silva et al., Cancer Immunol Res (2014) 2(5): 410-422), clone 6136E2, clone 024 (Sino Biological) clone 344801 (R&D Systems), clone E-18, clone H-191 (Santa Cruz Biotechnology), or clone 13A224 (United States Biological). In some embodiments, the antigen binding fragment for PD-1 may comprise the CDRs, light and heavy chain variable domains or other PD-1 binding fragment of e.g. anti-PD-1 antibody clone J116, clone MIH4 (eBioscience), clone 7A11B1 (Rockland Immunochemicals Inc.), clone 192106 (R&D Systems), clone J110, clone J105 (MBL International), clone 12A7D7, clone 7A11B1 (Abbiotec), clone #9X21 (MyBioSource.com), clone 4H4D1 (Proteintech Group), clone D3W4U, clone D3045 (Cell Signaling Technology), clone RMP1-30, clone RMP1-14 (Merck Millipore), clone EH12.2H7 (BioLegend), clone 1061227 (United States Biological), clone UMAB198, clone UMAB197 (Origene Technologies), nivolumab (BMS-936558), lambrolizumab, or anti-PD-1 antibodies described in WO 2010/077634 or WO 2006/121168. In some embodiments, the antigen binding fragment for KIR may comprise the CDRs, light and heavy chain variable domains or other KIR binding fragment of e.g. anti-KIR antibody clone 1-7F9 (Romagne et al., Blood (2009) 114(13): 2667-2677), lirilumab (BMS-986015; Sola et al., J Immunother Cancer (2013); 1:P40) or anti-KIR antibodies described in US 2015/0344576 or WO 2014/066532. In some embodiments, the antigen binding fragment for HER-2 may comprise the CDRs, light and heavy chain variable domains or other HER-2 binding fragment of e.g. anti-HER-2 antibody trastuzumab (Herceptin), or anti-HER-2 antibodies described in WO 2003/006509 or WO 2008/019290. In some embodiments, the antigen binding fragment for HER-3 may comprise the CDRs, light and heavy chain variable domains or other HER-3 binding fragment of e.g. anti-HER-3 antibody clone MM-121 (Lyu et al., Int. J Clin Exp Pathol (2015) 8(6): 6143-6156), MEHD7945A (Schaefer et al., Cancer Cell (2011) 20(4): 472-486), AMG 888 (U3-1287; Aurisicchio et al., Oncotarget (2012) 3(8): 744-758) or anti-HER-3 antibodies described in WO2008/100624 or WO 2013048883. In some embodiments, the antigen binding fragment for EGFR may comprise the CDRs, light and heavy chain variable domains or other EGFR binding fragment of e.g. anti-EGFR antibody panitumumab (ABX-EGF; Vectibix), cetuximab (Erbitux), nimotuzumab, matazumab (EMD 7200) or antibody clone 048-006 (Sogawa et al., Nucl Med Comm (2012) 33(7): 719-725). In some embodiments, the antigen binding fragment for EpCAM may comprise the CDRs, light and heavy chain variable domains or other EpCAM binding fragment of e.g. anti-EpCAM antibody edrecolomab, ING-1, 3622W4, or adecatumumab (Munz et al., Cancer Cell Int (2010) 10:44). In some embodiments, the antigen binding fragment for CD30 may comprise the CDRs, light and heavy chain variable domains or other CD30 binding fragment of e.g. anti-CD30 antibody brentuximab (cAC10), clone SGN-30 (Wahl et al., Cancer Res 2002 62(13):3736-3742), clone 5F11 (Borchmann et al., Blood (2003) 102(1): 3737-3742), or anti-CD30 antibodies described in WO 1993024135 or WO 2003059282. In some embodiments, the antigen binding fragment for CD33 may comprise the CDRs, light and heavy chain variable domains or other CD33 binding fragment of e.g. anti-CD33 antibody lintuzumab (SGN-33), gemtuzumab (Mylotarg), or clone hP67.7 (Sievers et al., Blood (1999) 93(11): 3678-3684). In some embodiments, the antigen binding fragment for CD38 may comprise the CDRs, light and heavy chain variable domains or other CD38 binding fragment of e.g. anti-CD38 antibody daratumumab (Darzalex), SAR650984 (Martin et al., J Clin Oncol (2014) 32:5s, (suppl; abstr 8532) or MOR202 (MorphoSys AG), or anti-CD38 antibodies described in WO 2006099875 or US 20100285004. In some embodiments, the antigen binding fragment for CD20 may comprise the CDRs, light and heavy chain variable domains or other CD20 binding fragment of e.g. anti-CD20 antibody rituximab, ocrelizumab, ofatumumab, obinutuzumab or BM-ca (Kobayashi et al., Cancer Med (2013) 2(2): 130-143). In some embodiments, the antigen binding fragment for CD24 may comprise the CDRs, light and heavy chain variable domains or other CD24 binding fragment of e.g. anti-CD24 antibody clone eBioSN3 (eBioscience), clone ML5 (BD Biosciences), or anti-CD24 antibodies described in WO 2008059491. In some embodiments, the antigen binding fragment for CD90 may comprise the CDRs, light and heavy chain variable domains or other CD90 binding fragment of e.g. anti-CD90 antibody clone 5E10 (BD Biosciences). In some embodiments, the antigen binding fragment for CD15 may comprise the CDRs, light and heavy chain variable domains or other CD15 binding fragment of e.g. anti-CD15 antibody clone C3D-1, Carb-3 (DAKO A/S), MMA (Roche) or BY87 (Abcam). In some embodiments, the antigen binding fragment for CD52 may comprise the CDRs, light and heavy chain variable domains or other CD52 binding fragment of e.g. anti-CD52 antibody alemtuzumab, clone HI186, or clone YTH34.5 (AbD Serotec). In some embodiments, the antigen binding fragment for CA-125 may comprise the CDRs, light and heavy chain variable domains or other CA-125 binding fragment of e.g. anti-CA-125 antibody oregovomab. In some embodiments, the antigen binding fragment for CD34 may comprise the CDRs, light and heavy chain variable domains or other CD34 binding fragment of e.g. anti-CD34 antibody clone 561 (BioLegend), clone 581 (Beckton Dickinson), or clone 5F3 (Sigma Aldrich). In some embodiments, the antigen binding fragment for CA-15-3 may comprise the CDRs, light and heavy chain variable domains or other CA-15-3 binding fragment of e.g. anti-CA-15-3 antibody clone 2F16 (USBiological), clone TA998 (ThermoFisher Scientific), clone 1 D1 (Sigma Aldrich), or Mab AR20.5 (Qi et al., Hybrid Hybridomics (2001) 20(5-6): 313-324). In some embodiments, the antigen binding fragment for CA-19-9 may comprise the CDRs, light and heavy chain variable domains or other CA-19-9 binding fragment of e.g. anti-CA-19-9 antibody clone 116-NS-19-9 (DAKO A/S), clone SPM110, or clone 121SLE (ThermoFisher Scientific). In some embodiments, the antigen binding fragment for CEA may comprise the CDRs, light and heavy chain variable domains or other CEA binding fragment of e.g. anti-CEA antibody labetuzumab, C2-45 (Kyowa Hakko Kirin Co. Ltd.) or anti-CEA antibodies disclosed in Imakiire et al., Int J Cancer (2004) 108: 564-570 or WO 2011034660. In some embodiments, the antigen binding fragment for CD99 may comprise the CDRs, light and heavy chain variable domains or other CD99 binding fragment of e.g. anti-CD99 antibody clone C7A (Moricoli et al., J Immunol Methods (2014) 408: 35-45) or clone 12E7 (DAKO A/S). In some embodiments, the antigen binding fragment for CD117 may comprise the CDRs, light and heavy chain variable domains or other CD117 binding fragment of e.g. anti-CD117 antibody clone CK6 (Lebron et al., Cancer Biol Ther (2014) 15(9): 1208-1218), or clone 104D2 (Sigma Aldrich). In some embodiments, the antigen binding fragment for CD31 may comprise the CDRs, light and heavy chain variable domains or other CD31 binding fragment of e.g. anti-CD31 antibody clone JC70A (DAKO A/S). In some embodiments, the antigen binding fragment for CD44 may comprise the CDRs, light and heavy chain variable domains or other CD44 binding fragment of e.g. anti-CD44 antibody PF-03475952 (Runnels et al., Adv Ther (2010); 27(3): 168-180), RG7356 (Vugts et al., MAbs (2014) 6(2): 567-575), clone IM7, or clone A3D8 (Sigma Aldrich). In some embodiments, the antigen binding fragment for CD123 may comprise the CDRs, light and heavy chain variable domains or other CD123 binding fragment of e.g. anti-CD123 antibody CSL362 (Nievergall et al., Blood (2014) 123(8):1218-1228), CSL360 (He et al., Leuk Lymphoma (2015) 56(5): 1406-1415) 73G (Jin et al., Cell Stem Cell (2009) 5(1): 31-42) clone 6H6 (AbD Serotec) or anti-CD123 antibodies described in WO 2014130635. In some embodiments, the antigen binding fragment for CD133 may comprise the CDRs, light and heavy chain variable domains or other CD133 binding fragment of e.g. anti-CD133 antibody clone 6B3, clone 9G4, clone AC141 (Wang et al., Hybridoma (Larchmt) (2010) 29(3): 241-249), clone 6B6 (Chen et al., Hybridoma (Larchmt) (2010) 29(4): 305-310, clone AC113 (Miltenyi Biotec), or anti-CD133 antibodies described in WO 2011149493. In some embodiments, the antigen binding fragment for ABCB5 may comprise the CDRs, light and heavy chain variable domains or other ABCB5 binding fragment of e.g. anti-ABCB5 antibody clone 5H3C6 (Thermo Fisher Scientific). In some embodiments, the antigen binding fragment for CD45 may comprise the CDRs, light and heavy chain variable domains or other CD45 binding fragment of e.g. anti-CD45 antibody YAML568 (Glatting et al., J Nucl Med (2006) 47(8): 1335-1341) or clone BRA-55 (Sigma Aldrich).

An antigen binding fragment of a bispecific antibody or bispecific antigen binding fragment according to the present invention may be any fragment of a polypeptide which is capable of binding to an antigen. In some embodiments, an antigen binding fragment comprises at least the three light chain CDRs (i.e. LC-CDR1, LC-CDR2 and LC-CDR3) and three heavy chain CDRs (i.e. HC-CDR1, HC-CDR2 and HC-CDR3) which together define the antigen binding region of an antibody or antigen binding fragment. In some embodiments, an antigen binding fragment may comprise the light chain variable domain and heavy chain variable domain of an antibody or antigen binding fragment. In some embodiments, an antigen binding fragment may comprise the light chain polypeptide and heavy chain polypeptide of an antibody or antigen binding fragment.

Bispecific antibodies and bispecific antigen binding fragments according to the invention may be provided in any suitable format, such as those formats described in Kontermann MAbs 2012, 4(2): 182-197, which is hereby incorporated by reference in its entirety. For example, a bispecific antibody or bispecific antigen binding fragment may be a bispecific antibody conjugate (e.g. an IgG2, F(ab′)₂ or CovX-Body), a bispecific IgG or IgG-like molecule (e.g. an IgG, seFv4-1g, IgG-scFv, scFv-IgG, DVD-Ig, IgG-sVD, sVD-IgG, 2 in 1-IgG, mAb², or Tandemab common LC), an asymmetric bispecific IgG or IgG-like molecule (e.g. a kih IgG, kih IgG common LC, CrossMab, kih IgG-seFab, mAb-Fv, charge pair or SEED-body), a small bispecific antibody molecule (e.g. a Diabody (Db), dsDb, DART, scDb, tandAbs, tandem scFv (taFv), tandem dAb/VHH, triple body, triple head, Fab-scFv, or F(ab′)₂-scFv₂), a bispecific Fc and C_{H}3 fusion protein (e.g. a taFv-Fc, Di-diabody, scDb-C_{H}3, scFv-Fc-scFv, HCAb-VHH, scFv-kih-Fc, or scFv-kih-C_{H}3), or a bispecific fusion protein (e.g. a scFv₂-albumin, scDb-albumin, taFv-toxin, DNL-Fab₃, DNL-Fab₄-IgG, DNL-Fab₄-IgG-cytokine₂). See in particular FIG. 2 of Kontermann MAbs 2012, 4(2): 182-19.

The skilled person is able to design and prepare bispecific antibodies and bispecific antigen binding fragments according to the present invention.

Methods for producing bispecific antibodies include chemically crosslinking of antibodies or antibody fragments, e.g. with reducible disulphide or non-reducible thioether bonds, for example as described in Segal and Bast, 2001. Production of Bispecific Antibodies. Current Protocols in Immunology. 14:IV:2.13:2.13.1-2.13.16, which is hereby incorporated by reference in its entirety. For example, N-succinimidyl-3-(-2-pyridyldithio)-propionate (SPDP) can be used to chemically crosslink e.g. Fab fragments via hinge region SH— groups, to create disulfide-linked bispecific F(ab)₂ heterodimers.

Other methods for producing bispecific antibodies include fusing antibody-producing hybridomas e.g. with polyethylene glycol, to produce a quadroma cell capable of secreting bispecific antibody, for example as described in D. M. and Bast, B. J. 2001. Production of Bispecific Antibodies. Current Protocols in Immunology. 14:IV:2.13:2.13.1-2.13.16.

Bispecific antibodies and bispecific antigen binding fragments according to the present invention can also be produced recombinantly, by expression from e.g. a nucleic acid construct encoding polypeptides for the antigen binding molecules, for example as described in Antibody Engineering: Methods and Protocols, Second Edition (Humana Press, 2012), at Chapter 40: Production of Bispecific Antibodies: Diabodies and Tandem scFv (Hornig and Färber-Schwarz), or French, How to make bispecific antibodies, Methods Mol. Med. 2000; 40:333-339, the entire contents of both of which are hereby incorporated by reference. For example, a DNA construct encoding the light and heavy chain variable domains for the two antigen binding fragments (i.e. the light and heavy chain variable domains for the antigen binding fragment capable of binding LAG-3, and the light and heavy chain variable domains for the antigen binding fragment capable of binding to another target protein), and including sequences encoding a suitable linker or dimerization domain between the antigen binding fragments can be prepared by molecular cloning techniques. Recombinant bispecific antibody can thereafter be produced by expression (e.g. in vitro) of the construct in a suitable host cell (e.g. a mammalian host cell), and expressed recombinant bispecific antibody can then optionally be purified.

Antibodies may be produced by a process of affinity maturation in which a modified antibody is generated that has an improvement in the affinity of the antibody for antigen, compared to an unmodified parent antibody. Affinity-matured antibodies may be produced by procedures known in the art, e.g., Marks et al., Rio/Technology 10:779-783 (1992); Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):331 0-15 9 (1995); and Hawkins et al, J. Mol. Biol. 226:889-896 (1992).

Antibodies according to the present invention preferably exhibit specific binding to LAG-3. An antibody that specifically binds to a target molecule preferably binds the target with greater affinity, and/or with greater duration than it binds to other targets. In some embodiments the present antibodies may bind with greater affinity to LAG-3 than to one or more of PD-1, TIM-3, ICOS, BTLA, CD28 or CTLA-4. In one embodiment, the extent of binding of an antibody to an unrelated target is less than about 10% of the binding of the antibody to the target as measured, e.g., by ELISA, SPR, Bio-Layer Interferometry or by a radioimmunoassay (RIA). Alternatively, the binding specificity may be reflected in terms of binding affinity where the anti-LAG-3 antibody of the present invention binds to LAG-3 with a K_{D} that is at least 0.1 order of magnitude (i.e. 0.1×10ⁿ, where n is an integer representing the order of magnitude) greater than the K_{D} of the antibody towards another target molecule. This may optionally be one of at least 0.2, 0.3, 0.4, 0.5, 0.6, 0.7, 0.8, 0.9, 1.0, 1.5, or 2.0.

Antibodies according to the present invention preferably have a dissociation constant (K_{D}) of one of ≤10 nM, ≤5 nM, ≤3 nM, ≤2 nM, ≤1.5 nM, ≤1.4 nM, ≤1.3 nM, ≤1.25 nM, ≤1.24 nM, ≤1.23 nM, ≤1.22 nM, ≤1.21 nM, ≤1.2 nM, ≤1.15 nM, ≤1.1 nM ≤1.05 nM, ≤1 nM, ≤900 pM, ≤800 pM, ≤700 pM, ≤600 pM, ≤500 pM. The K_{D} may be in the range about 0.1 to about 3 nM. Binding affinity of an antibody for its target is often described in terms of its dissociation constant (K_{D}). Binding affinity can be measured by methods known in the art, such as by ELISA, Surface Plasmon Resonance (SPR; see e.g. Hearty et al., Methods Mol Biol (2012) 907:411-442), Bio-Layer Interferometry (see e.g. Lad et al., (2015) J Biomol Screen 20(4): 498-507), or by a radiolabeled antigen binding assay (RIA) performed with the Fab version of the antibody and antigen molecule.

Antibodies according to the present invention preferably exhibit binding to LAG-3 (e.g. human LAG-3) with greater affinity than, or with similar affinity to, affinity of binding by BMS-986016 (described, for example, in WO 2015042246—A1 SEQ ID NOs: 1 and 2 of WO 2015042246 A1 are respectively the heavy and light chain amino acid sequences for BMS-986016).

As used herein, an antibody displaying ‘greater affinity’ for a given target molecule compared to a reference antibody binds to that target molecule with greater strength as compared to the strength of binding of the reference antibody to the target molecule. The affinity of an antibody for a given target molecule can be determined quantitatively.

Relative affinity of binding of an antibody according to the invention to LAG-3 compared to BMS-986016 can be determined for example by ELISA, as described herein. In some embodiments, an antibody according to the present invention may have a dissociation constant (K_{D}) for LAG-3 which is less than or equal to the K_{D} of BMS-986016 for LAG-3.

In some embodiments, an antibody according to the present invention may have affinity for LAG-3 which is 1.01 times or greater, 1.05 times or greater, 1.1 times or greater, 1.15 times or greater, 1.2 times or greater, 1.25 times or greater, 1.3 times or greater, 1.35 times or greater, 1.4 times or greater, 1.45 times or greater, 1.5 times or greater than the affinity of BMS-986016 for LAG-3, in a given assay. In some embodiments, an antibody to according to the present invention may bind to LAG-3 with a K_{D} value which is 0.99 times or less, 0.95 times or less, 0.9 times or less, 0.85 times or less, 0.8 times or less, 0.75 times or less, 0.7 times or less, 0.65 times or less, 0.6 times or less, 0.55 times or less, 0.5 times or less of the K_{D} value of BMS-986016 for LAG-3, in a given assay.

Antibodies according to the present invention preferably inhibit or prevent interaction between LAG-3 and MHC class II (e.g. human LAG-3 and human MHC class II) to a greater extent than, or to a similar extent to, inhibition/prevention of interaction between LAG-3 and MHC class II by BMS-986016. Relative inhibition/prevention of interaction between LAG-3 and LAG-3 of an antibody according to the invention for LAG-3 compared to BMS-986016 can be determined for example as described in Example 8 herein.

For example, relative inhibition/prevention of interaction between LAG-3 and MHC class II of an antibody according to the invention compared to BMS-986016 can be determined for example as described herein. Briefly, inhibition/prevention of interaction by a given antibody can be evaluated by pre-incubating labelled (e.g. fluorescently-labelled) LAG-3 with the antibody, subsequently applying the pre-mix to cells expressing MHC class II (e.g. Daudi cells), incubating the pre-mix and cells for sufficient time to allow binding of LAG-3 to MHC class II, washing to remove unbound LAG-3 and LAG-3-antibody complexes, and finally analysing the cells to detect the label.

In some embodiments, an antibody according to the present invention may inhibit/prevent interaction between LAG-3 and MHC class II to an extent which is greater than or equal to inhibition/prevention of interaction between LAG-3 and MHC class II by BMS-986016. In some embodiments, an antibody according to the present invention may inhibit/prevent interaction between LAG-3 and MHC class II to an extent which is 1.01 times or greater, 1.05 times or greater, 1.1 times or greater, 1.15 times or greater, 1.2 times or greater, 1.25 times or greater, 1.3 times or greater, 1.35 times or greater, 1.4 times or greater, 1.45 times or greater, 1.5 times or greater than inhibition/prevention of interaction between LAG-3 and MHC class II by BMS-986016, in a given assay.

In some embodiments, an antibody according to the present invention may inhibit/prevent interaction between LAG-3 and MHC class II with a value for half maximal inhibition of interaction (i.e. an IC₅₀ value for inhibition of interaction between LAG-3 and MHC class II) which is lower than the IC₅₀ value for inhibition of interaction between LAG-3 and MHC class II by BMS-986016. In some embodiments, an antibody according to the present invention may inhibit/prevent interaction between LAG-3 and MHC class II with an IC₅₀ value which is 0.99 times or less, 0.95 times or less, 0.9 times or less, 0.85 times or less, 0.8 times or less, 0.75 times or less, 0.7 times or less, 0.65 times or less, 0.6 times or less, 0.55 times or less, 0.5 times or less of the IC₅₀ value for inhibition of interaction between LAG-3 and MHC class II by BMS-986016, in a given assay.

Antibodies according to the present invention preferably increase one or more of T-cell proliferation, IL-2 production and IFNγ production in a Mixed Lymphocyte Reaction (MLR) assay. MLR assays may be performed as described in Bromelow et al J. Immunol Methods, 2001 Jan. 1; 247(1-2):1-8. T cell proliferation may be evaluated by methods well known to the skilled person, such as by measuring incorporation of tritiated thymidine or by CFSE dye dilution, e.g. as described in Anthony et al., 2012 Cells 1:127-140. IL-2 and/or IFNγ production may be analysed e.g. by antibody-based methods well known to the skilled person, such as western blot, immunohistochemistry, immunocytochemistry, flow cytometry, ELISA, ELISPOT, or by reporter-based methods.

In some embodiments, an antibody according to the present invention may increase one or more of T-cell proliferation, IL-2 production and IFNγ production in a MLR assay to a similar extent to, or to a greater extent than, BMS-986016. In some embodiments, an antibody according to the present invention may increase one or more of T-cell proliferation, IL-2 production and IFNγ production in a MLR assay to an extent which is 1.01 times or greater, 1.05 times or greater, 1.1 times or greater, 1.15 times or greater, 1.2 times or greater, 1.25 times or greater, 1.3 times or greater, 1.35 times or greater, 1.4 times or greater, 1.45 times or greater, 1.5 times or greater than increase in T-cell proliferation, IL-2 production and IFNγ production in a MLR assay in response to BMS-986016, in a comparable assay.

Antibodies according to the present invention may bind to an epitope of LAG-3 which is different to the epitope of LAG-3 to which BMS-986016 binds. In some embodiments, the epitope for an antibody according to the present invention does not overlap with the epitope of LAG-3 to which BMS-986016 binds. In some embodiments, an antibody according to the present invention does not compete with BMS-986016 for binding to LAG-3.

The epitope of LAG-3 to which a given antibody binds can be determined by methods well known to the skilled person, including by X-ray crystallography, array-based oligopeptide scanning, mutagenesis-based mapping and hydrogen-deuterium exchange mapping methods. Competitive binding for an epitope can be analysed by competition ELISA or by binding response analysis e.g. using SPR, or by Bio-Layer Interferometry as described herein.

Antibodies according to the present invention may be “antagonist” antibodies that inhibit or reduce a biological activity of the antigen to which it binds. Blocking of interaction between LAG-3 and MHC class II assists in the restoration of T-cell function by inhibiting the immune-inhibitory signalling pathway mediated by LAG-3.

The present invention also provides a chimeric antigen receptor (CAR) comprising an antigen binding fragment according to the present invention.

Chimeric Antigen Receptors (CARs) are recombinant receptors that provide both antigen-binding and T cell activating functions. CAR structure and engineering is reviewed, for example, in Dotti et al., Immunol Rev (2014) 257(1), hereby incorporated by reference in its entirety.

CARs comprise an antigen-binding region linked to a cell membrane anchor region and a signaling region. An optional hinge region may provide separation between the antigen-binding region and cell membrane anchor region, and may act as a flexible linker.

The antigen-binding region of a CAR may be based on the antigen-binding region of an antibody which is specific for the antigen to which the CAR is targeted, or other agent capable of binding to the target. For example, the antigen-binding domain of a CAR may comprise amino acid sequences for the complementarity-determining regions (CDRs) or complete light chain and heavy chain variable region amino acid sequences of an antibody which binds specifically to the target protein. Antigen-binding domains of CARs may target antigen based on other protein:protein interaction, such as ligand:receptor binding; for example an IL-13Rα2-targeted CAR has been developed using an antigen-binding domain based on IL-13 (see e.g. Kahlon et al. 2004 Cancer Res 64(24): 9160-9166).

The CAR of the present invention comprises a LAG-3 binding region. In some embodiments, the CAR of the present invention comprises an antigen binding region which comprises or consists of an antibody/antigen binding fragment according to the present invention.

The LAG-3 binding region of the CAR of the present invention may be provided with any suitable format, e.g. scFv, Fab, etc. In some embodiments, the LAG-3 binding region of the CAR of the present invention comprises or consists of a LAG-3 binding scFv.

The cell membrane anchor region is provided between the antigen-binding region and the signalling region of the CAR. The cell membrane anchor region provides for anchoring the CAR to the cell membrane of a cell expressing a CAR, with the antigen-binding region in the extracellular space, and signalling region inside the cell. In some embodiments, the CAR of the present invention comprises a cell membrane anchor region comprising or consisting of an amino acid sequence which comprises, consists of, or is derived from, the transmembrane region amino acid sequence for one of CD3-ζ, CD4, CD8 or CD28.

As used herein, a region which is ‘derived from’ a reference amino acid sequence comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the reference sequence.

The signalling region of a CAR allows for activation of the T cell. The CAR signalling regions may comprise the amino acid sequence of the intracellular domain of CD3-ζ, which provides immunoreceptor tyrosine-based activation motifs (ITAMs) for phosphorylation and activation of the CAR-expressing T cell. Signalling regions comprising sequences of other ITAM-containing proteins have also been employed in CARs, such as domains comprising the ITAM containing region of FcγRI (Haynes et al., 2001 J Immunol 166(1):182-187). CARs comprising a signalling region derived from the intracellular domain of CD3-ζ are often referred to as first generation CARs.

Signalling regions of CARs may also comprise co-stimulatory sequences derived from the signalling region of co-stimulatory molecules, to facilitate activation of CAR-expressing T cells upon binding to the target protein. Suitable co-stimulatory molecules include CD28, OX40, 4-1BB, ICOS and CD27. CARs having a signalling region including additional co-stimulatory sequences are often referred to as second generation CARs.

In some cases CARs are engineered to provide for co-stimulation of different intracellular signalling pathways. For example, signalling associated with CD28 costimulation preferentially activates the phosphatidylinositol 3-kinase (P13K) pathway, whereas the 4-1BB-mediated signalling is through TNF receptor associated factor (TRAF) adaptor proteins. Signalling regions of CARs therefore sometimes contain co-stimulatory sequences derived from signalling regions of more than one co-stimulatory molecule. CARs comprising a signalling region with multiple co-stimulatory sequences are often referred to as third generation CARs.

In some embodiments, the CAR of the present invention comprises one or more co-stimulatory sequences comprising or consisting of an amino acid sequence which comprises, consists of, or is derived from, the amino acid sequence of the intracellular domain of one or more of CD28, OX40, 4-1BB, ICOS and CD27.

An optional hinge region may provide separation between the antigen-binding domain and the transmembrane domain, and may act as a flexible linker. Hinge regions may be flexible domains allowing the binding moiety to orient in different directions. Hinge regions may be derived from IgG1 or the CH2CH3 region of immunoglobulin. In some embodiments, the CAR of the present invention comprises a hinge region comprising or consisting of an amino acid sequence which comprises, consists of, or is derived from, the amino acid sequence of the hinge region of IgG1 or the CH2CH3 region of immunoglobulin.

CARs may be combined with costimulatory ligands, chimeric costimulatory receptors or cytokines to further enhance T cell potency, specificity and safety (Sadelain et al., The basic principles of chimeric antigen receptor (CAR) design. Cancer Discov. 2013 April; 3(4): 388-398. doi:10.1158/2159-8290.CD-12-0548, specifically incorporated herein by reference).

Also provided is a cell comprising a CAR according to the invention. The CAR according to the present invention may be used to generate T cells. Engineering of CARs into T cells may be performed during culture, in vitro, for transduction and expansion, such as happens during expansion of T cells for adoptive T cell therapy.

In some aspects, the antibody is clone A6, or a variant of A6. A6 comprises the following CDR sequences:

Light Chain:

(SEQ ID NO: 12)

LC-CDR1: RSSQSLLHSNGYNYLD

(SEQ ID NO: 13)

LC-CDR2: LGSNRAS

(SEQ ID NO: 14)

LC-CDR3: MQALQTPYT

Heavy Chain:

(SEQ ID NO: 28)

HC-CDR1: SYYMH

(SEQ ID NO: 29)

HC-CDR2: IINPSGGSTSYAQKFQG

(SEQ ID NO: 30)

HC-CDR3: PFGDFDY.

CDR Sequences Determined by Kabat Definition.

In some aspects, the antibody is clone 1G11, or a variant of 1G11. 1G11 comprises the following CDR sequences:

Light Chain:

(SEQ ID NO: 15)

LC-CDR1: RASQSVSSSFLA

(SEQ ID NO: 16)

LC-CDR2: GASSRAT

(SEQ ID NO: 17)

LC-CDR3: QQYGPSIT

Heavy Chain:

(SEQ ID NO: 31)

HC-CDR1: SYGMH

(SEQ ID NO: 32)

HC-CDR2: VISYDGSNKYYADSVKG

(SEQ ID NO: 33)

HC-CDR3: LPGWGAYAFDI.

CDR Sequences Determined by Kabat Definition.

In some aspects, the antibody is clone C2, or a variant of C2. C2 comprises the following CDR sequences:

Light Chain:

(SEQ ID NO: 18)

LC-CDR1: RASQSVSSSYLA

(SEQ ID NO: 16)

LC-CDR2: GASSRAT

(SEQ ID NO: 19)

LC-CDR3: QQYGSSPPIT

Heavy Chain:

(SEQ ID NO: 34)

HC-CDR1: SYAMH

(SEQ ID NO: 32)

HC-CDR2: VISYDGSNKYYADSVKG

(SEQ ID NO: 35)

HC-CDR3: DPDAANWGFLLYYGMDV.

CDR Sequences Determined by Kabat Definition.

In some aspects, the antibody is clone C12, or a variant of C12. C12 comprises the following CDR sequences:

Light Chain:

(SEQ ID NO: 20)

LC-CDR1: RSSQSLLHSDGYNYFD

(SEQ ID NO: 21)

LC-CDR2: LGSNRAA

(SEQ ID NO: 22)

LC-CDR3: MQGTHWPPT

Heavy Chain:

(SEQ ID NO: 36)

HC-CDR1: SYAIS

(SEQ ID NO: 37)

HC-CDR2: GIIPIFGTANYAQKFQG

(SEQ ID NO: 38)

HC-CDR3: ALADFWSGYYYYYYMDV.

CDR Sequences Determined by Kabat Definition.

In some aspects, the antibody is clone F5, or a variant of F5. F5 comprises the following CDR sequences:

Light Chain:

(SEQ ID NO: 23)

LC-CDR1: RASQSVSSGYLA

(SEQ ID NO: 24)

LC-CDR2: DASSRAT

(SEQ ID NO: 25)

LC-CDR3: QQYGSSRPGLT

Heavy Chain:

(SEQ ID NO: 39)

HC-CDR1: ELSMH

(SEQ ID NO: 40)

HC-CDR2: GFDPEDGETIYAQKFQG

(SEQ ID NO: 41)

HC-CDR3: TWFGELYY.

CDR Sequences Determined by Kabat Definition.

In some aspects, the antibody is clone G8, or a variant of G8. G8 comprises the following CDR sequences:

Light Chain:

(SEQ ID NO: 26)

LC-CDR1: TTSQSVSSTSLD

(SEQ ID NO: 16)

LC-CDR2: GASSRAT

(SEQ ID NO: 27)

LC-CDR3: QQYGSSLLT

Heavy Chain:

(SEQ ID NO: 34)

HC-CDR1: SYAMH

(SEQ ID NO: 32)

HC-CDR2: VISYDGSNKYYADSVKG

(SEQ ID NO: 35)

HC-CDR3: DPDAANWGFLLYYGMDV.

CDR Sequences Determined by Kabat Definition.

Antibodies according to the present invention may comprise the CDRs of A6, 1G11, C2, C12, F5 or G8 or one of SEQ ID NOs 1 and 7; 2 and 8; 3 and 9; 4 and 10; 5 and 11; or 6 and 9. In an antibody according to the present invention one or two or three or four of the six CDR sequences may vary. A variant may have one or two amino acid substitutions in one or two of the six CDR sequences.

Amino acid sequences of the V_{H} and V_{L} chains of anti-LAG-3 clones are shown in FIGS. 1 and 2. The encoding nucleotide sequences are shown in FIG. 4.

The light and heavy chain CDRs may also be particularly useful in conjunction with a number of different framework regions. Accordingly, light and/or heavy chains having LC-CDR1-3 or HC-CDR1-3 may possess an alternative framework region. Suitable framework regions are well known in the art and are described for example in M. Lefranc & G. Le:franc (2001) “The Immunoglobulin FactsBook”, Academic Press, incorporated herein by reference.

In this specification, antibodies may have V_{H} and/or V_{L} chains comprising an amino acid sequence that has a high percentage sequence identity to one or more of the V_{H} and/or V_{L} amino acid sequences of SEQ ID NOs 1 and 7; 2 and 8; 3 and 9; 4 and 10; 5 and 11; or 6 and 9, or to one or the amino acid sequences shown in FIGS. 1 and 2.

For example, antibodies according to the present invention include antibodies that bind LAG-3 and have a V_{H} or V_{L} chain that comprises an amino acid sequence having at least 70%, more preferably one of at least 75%, 80%, 85%, 86%, 87%, 88%, 89%, 90%, 91%, 92%, 93%, 94%, 95%, 96%, 97%, 98%, 99%, or 100%, sequence identity to the V_{H} or V_{L} chain amino acid sequence of one of SEQ ID NOs 1 to 11, or to one or the amino acid sequences shown in FIGS. 1 and 2.

Antibodies according to the present invention may be detectably labelled or, at least, capable of detection. For example, the antibody may be labelled with a radioactive atom or a coloured molecule or a fluorescent molecule or a molecule which can be readily detected in any other way. Suitable detectable molecules include fluorescent proteins, luciferase, enzyme substrates, and radiolabels. The binding moiety may be directly labelled with a detectable label or it may be indirectly labelled. For example, the binding moiety may be an unlabelled antibody which can be detected by another antibody which is itself labelled. Alternatively, the second antibody may have bound to it biotin and binding of labelled streptavidin to the biotin is used to indirectly label the first antibody.

Nucleic Acids/Vectors

The present invention provides a nucleic acid encoding an antibody, antigen binding fragment or CAR according to the present invention. In some embodiments, the nucleic acid is purified or isolated, e.g. from other nucleic acid, or naturally-occurring biological material.

The present invention also provides a vector comprising nucleic acid encoding an antibody, antigen binding fragment or CAR according to the present invention.

The nucleic acid and/or vector according to the present invention may be provided for introduction into a cell, e.g. a primary human immune cell. Suitable vectors include plasmids, binary vectors, DNA vectors, mRNA vectors, viral vectors (e.g. gammaretroviral vectors (e.g. murine Leukemia virus (MLV)-derived vectors), lentiviral vectors, adenovirus vectors, adeno-associated virus vectors, vaccinia virus vectors and herpesvirus vectors), transposon-based vectors, and artificial chromosomes (e.g. yeast artificial chromosomes), e.g. as described in Maus et al., Annu Rev Immunol (2014) 32:189-225 or Morgan and Boyerinas, Biomedicines 2016 4, 9, which are both hereby incorporated by reference in its entirety. In some embodiments, the viral vector may be a lentiviral, retroviral, adenoviral, or Herpes Simplex Virus vector. In some embodiments, the lentiviral vector may be pELNS, or may be derived from pELNS. In some embodiments, the vector may be a vector encoding CRISPR/Cas9.

Cells Comprising/Expressing the Antibodies/Fragments/CARs

The present invention also provides a cell comprising or expressing an antibody, antigen binding fragment or CAR, according to the present invention. Also provided is a cell comprising or expressing a nucleic acid or vector according to the invention.

The cell may be a eukaryotic cell, e.g. a mammalian cell. The mammal may be a human, or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate).

In some embodiments, the cell may be from, or may have been obtained from, a human subject.

The cell may be an immune cell. The cell may be a cell of hematopoietic origin, e.g. a neutrophil, eosinophil, basophil, dendritic cell, lymphocyte, or monocyte. The lymphocyte may be e.g. a T cell, B cell, NK cell, NKT cell or innate lymphoid cell (ILC), or a precursor thereof. The cell may express e.g. CD3 polypeptides (e.g. CD3γ CD3ε CD3ζ or CD3δ), TCR polypeptides (TCRα or TCRβ), CD27, CD28, CD4 or CD8. In some embodiments, the cell is a T cell. In some embodiments, the T cell is a CD3+ T cell. In some embodiments, the T cell is a CD3+, CD8+ T cell. In some embodiments, the T cell is a cytotoxic T cell (e.g. a cytotoxic T lymphocyte (CTL)).

Where the cell is a T cell comprising a CAR according to the present invention, the cell may be referred to as a CAR-T cell.

In some embodiments, the cell is an antigen-specific T cell. In embodiments herein, a “antigen-specific” T cell is a cell which displays certain functional properties of a T cell in response to the antigen for which the T cell is specific, or a cell expressing said antigen. In some embodiments, the properties are functional properties associated with effector T cells, e.g. cytotoxic T cells. In some embodiments, an antigen-specific T cell may display one or more of the following properties: cytotoxicity, e.g. to a cell comprising/expressing antigen for which the T cell is specific; proliferation, IFNγ expression, CD107a expression, IL-2 expression, TNFα expression, perforin expression, granzyme expression, granulysin expression, and/or FAS ligand (FASL) expression, e.g. in response to antigen for which the T cell is specific or a cell comprising/expressing antigen for which the T cell is specific. In some embodiments, the antigen for which the T cell is specific may be a peptide or polypeptide of a virus, e.g. Epstein-Barr virus (EBV), influenza virus, measles virus, hepatitis B virus (HBV), hepatitis C virus (HCV), human immunodeficiency virus (HIV), lymphocytic choriomeningitis virus (LCMV), Herpes simplex virus (HSV) or human papilloma virus (HPV).

The present invention also provides a method for producing a cell comprising a nucleic acid or vector according to the present invention, comprising introducing a nucleic acid or vector according to the present invention into a cell. The present invention also provides a method for producing a cell expressing an antibody, antigen binding fragment or CAR, according to the present invention, comprising introducing a nucleic acid or vector according to the present invention in a cell. In some embodiments, the methods additionally comprise culturing the cell under conditions suitable for expression of the nucleic acid or vector by the cell. In some embodiments, the methods are performed in vitro.

In some embodiments, introducing an isolated nucleic acid or vector according to the invention into a cell comprises transduction, e.g. retroviral transduction. Accordingly, in some embodiments the isolated nucleic acid or vector is comprised in a viral vector, or the vector is a viral vector. In some embodiments, the method comprises introducing a nucleic acid or vector according to the invention by electroporation, e.g. as described in Koh et al., Molecular Therapy—Nucleic Acids (2013) 2, e114, which is hereby incorporated by reference in its entirety.

The present invention also provides cells obtained or obtainable by the methods for producing a cell according to the present invention.

Methods of Detection

Antibodies, antigen binding fragments, CARs or cells described herein may be used in methods that involve the binding of the antibody, antigen binding fragment, CAR or cell to LAG-3. Such methods may involve detection of the bound complex of antibody, antigen binding fragment, CAR or cell and LAG-3. As such, in one embodiment a method is provided, the method comprising contacting a sample containing, or suspected to contain, LAG-3 with an antibody. antigen binding fragment, CAR or cell as described herein and detecting the formation of a complex of antibody, antigen binding fragment, CAR or cell and LAG-3.

Suitable method formats are well known in the art, including immunoassays such as sandwich assays, e.g. ELISA. The method may involve labelling the antibody, antigen binding fragment, CAR or cell, or LAG-3, or both, with a detectable label, e.g. fluorescent, luminescent or radio-label. LAG-3 expression may be measured by immunohistochemistry (IHC), for example of a tissue sample obtained by biopsy.

Methods of this kind may provide the basis of a method of diagnosis of a disease or condition requiring detection and or quantitation of LAG-3 or MHC class II. Such methods may be performed in vitro on a patient sample, or following processing of a patient sample. Once the sample is collected, the patient is not required to be present for the in vitro method of diagnosis to be performed and therefore the method may be one which is not practised on the human or animal body.

Such methods may involve determining the amount of LAG-3 present in a patient sample. The method may further comprise comparing the determined amount against a standard or reference value as part of the process of reaching a diagnosis. Other diagnostic tests may be used in conjunction with those described here to enhance the accuracy of the diagnosis or prognosis or to confirm a result obtained by using the tests described here.

Cancer cells may exploit the LAG-3 pathway to create an immunosuppressive environment, by upregulating expression of LAG-3, allowing activation of the inhibitory LAG-3 receptor on any T cells that infiltrate the tumor microenvironment and thereby suppressing their activity. Upregulation of LAG-3 expression has been demonstrated in many different cancer types, and high LAG-3 expression has also been linked to poor clinical outcomes.

The level of LAG-3 or MHC class II present in a patient sample may be indicative that a patient may respond to treatment with an anti-LAG-3 antibody. The presence of a high level of LAG-3 or MHC class II in a sample may be used to select a patient for treatment with an anti-LAG-3 antibody. The antibodies of the present invention may therefore be used to select a patient for treatment with anti-LAG-3 therapy.

Detection in a sample of LAG-3 may be used for the purpose of diagnosis of a T-cell dysfunctional disorder or a cancerous condition in the patient, diagnosis of a predisposition to a cancerous condition or for providing a prognosis (prognosticating) of a cancerous condition. The diagnosis or prognosis may relate to an existing (previously diagnosed) cancerous condition, which may be benign or malignant, may relate to a suspected cancerous condition or may relate to the screening for cancerous conditions in the patient (which may be previously undiagnosed).

In one embodiment the level of LAG-3 expression on CD8+ T cells may be detected in order to indicate the degree of T-cell exhaustion and severity of the disease state.

In one embodiment the level of MHC class II expression, e.g. on antigen presenting cells or tumor cells, may be detected in order to indicate existence or severity of a disease state, for example infection, tissue inflammation or a cancer.

A sample may be taken from any tissue or bodily fluid. The sample may comprise or may be derived from: a quantity of blood; a quantity of serum derived from the individual's blood which may comprise the fluid portion of the blood obtained after removal of the fibrin clot and blood cells; a tissue sample or biopsy; or cells isolated from said individual.

Methods according to the present invention are preferably performed in vitro. The term “in vitro” is intended to encompass experiments with cells in culture whereas the term “in vivo” is intended to encompass experiments with intact multi-cellular organisms.

Therapeutic Applications

Antibodies, antigen binding fragments, CARs, cells and polypeptides according to the present invention and compositions comprising such agents may be provided for use in methods of medical treatment. Treatment may be provided to subjects having a disease or condition in need of treatment. The disease or condition may be one of a T-cell dysfunctional disorder, including a T-cell dysfunctional disorder associated with a cancer, or a cancer, or a T-cell dysfunctional disorder associated with an infection, or an infection.

A T-cell dysfunctional disorder may be a disease or condition in which normal T-cell function is impaired causing downregulation of the subject's immune response to pathogenic antigens, e.g. generated by infection by exogenous agents such as microorganisms, bacteria and viruses, or generated by the host in some disease states such as in some forms of cancer (e.g. in the form of tumor associated antigens).

The T-cell dysfunctional disorder may comprise T-cell exhaustion or T-cell anergy. T-cell exhaustion comprises a state in which CD8⁺ T-cells fail to proliferate or exert T-cell effector functions such as cytotoxicity and cytokine (e.g. IFNγ) secretion in response to antigen stimulation. Exhausted T-cells may also be characterised by sustained expression of LAG-3, where blockade of LAG-3:MHC class II interactions may reverse the T-cell exhaustion and restore antigen-specific T cell responses.

The T-cell dysfunctional disorder may be manifest as an infection, or inability to mount an effective immune response against an infection. The infection may be chronic, persistent, latent or slow, and may be the result of bacterial, viral, fungal or parasitic infection. As such, treatment may be provided to patients having a bacterial, viral or fungal infection. Examples of bacterial infections include infection with Helicobacter pylori. Examples of viral infections include infection with HIV, hepatitis B or hepatitis C.

The T-cell dysfunctional disorder may be associated with a cancer, such as tumor immune escape. Many human tumors express tumor-associated antigens recognised by T cells and capable of inducing an immune response. Woo et al. Cancer Res (2012) 72(4): 917-927 describes regulation of T cell function by synergistic action of LAG-3 and PD-1 to promote tumoral immune escape in mice. Blocking the interaction of LAG-3 and MHC class II may inhibit this negative immunoregulatory signal to tumor cells and enhance tumor-specific CD8⁺ T-cell immunity.

Cancers may also be treated where there is no indication of a T-cell dysfunctional disorder such as T-cell exhaustion but the use of an antibody, antigen binding fragment, CAR, cell or polypeptide according to the present invention allows the subject to suppress LAG-3 signalling and mount an effective immune response with limited impairment, evasion or induction of tumor immune escape. In such treatments, the antibody, antigen binding fragment, CAR, cell or polypeptide may provide a treatment for cancer that involves prevention of the development of tumor immune escape.

Cancers may also be treated which overexpress LAG-3. For example, such tumor cells overexpressing LAG-3 may be killed directly by treatment with anti-LAG-3 antibodies, by antibody dependent cell-mediated cytotoxicity (ADCC), complement dependent cytotoxicity (CDC), or using anti-LAG-3 antibody-drug conjugates.

The treatment may be aimed at prevention of the T-cell dysfunctional disorder, e.g. prevention of infection or of the development or progression of a cancer. As such, the antibodies, antigen binding fragments, CARs, cells and polypeptides may be used to formulate pharmaceutical compositions or medicaments and subjects may be prophylactically treated against development of a disease state. This may take place before the onset of symptoms of the disease state, and/or may be given to subjects considered to be at greater risk of infection or development of cancer.

Treatment may comprise co-therapy with a vaccine, e.g. T-cell vaccine, which may involve simultaneous, separate or sequential therapy, or combined administration of vaccine and antibody, antigen binding fragment, CAR, cell or polypeptide in a single composition. In this context, the antibody, antigen binding fragment, CAR, cell or polypeptide may be provided as an adjuvant to the vaccine. Limited proliferative potential of exhausted T cells has been attributed as a main reason for failure of T-cell immunotherapy, and the combination of an agent capable of blocking or reversing T cell exhaustion is a potential strategy for improving the efficacy of T-cell immunotherapy (Barber et al., Nature Vol 439, No. 9 p 682-687 February 2006).

Administration of an antibody, antigen binding fragment, CAR, cell or polypeptide is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Formulating Pharmaceutically Useful Compositions and Medicaments

Antibodies, antigen binding fragments, CARs, cells and polypeptides according to the present invention may be formulated as pharmaceutical compositions for clinical use and may comprise a pharmaceutically acceptable carrier, diluent, excipient or adjuvant.

In accordance with the present invention methods are also provided for the production of pharmaceutically useful compositions, such methods of production may comprise one or more steps selected from: isolating an antibody, antigen binding fragment, CAR, cell or polypeptide as described herein; and/or mixing an isolated antibody, antigen binding fragment, CAR, cell or polypeptide as described herein with a pharmaceutically acceptable carrier, adjuvant, excipient or diluent.

For example, a further aspect of the present invention relates to a method of formulating or producing a medicament or pharmaceutical composition for use in the treatment of a T-cell dysfunctional disorder, the method comprising formulating a pharmaceutical composition or medicament by mixing an antibody, antigen binding fragment, CAR, cell or polypeptide as described herein with a pharmaceutically acceptable carrier, adjuvant, excipient or diluent.

Infection

An infection may be any infection or infectious disease, e.g. bacterial, viral, fungal, or parasitic infection. In some embodiments it may be particularly desirable to treat chronic/persistent infections, e.g. where such infections are associated with T cell dysfunction or T cell exhaustion.

It is well established that T cell exhaustion is a state of T cell dysfunction that arises during many chronic infections (including viral, bacterial and parasitic), as well as in cancer (Wherry Nature Immunology Vol. 12, No. 6, p 492-499, June 2011).

An infection or infectious disease may be one in which LAG-3 is upregulated.

Examples of bacterial infections that may be treated include infection by Bacillus spp., Bordetella pertussis, Clostridium spp., Corynebacterium spp., Vibrio chloerae, Staphylococcus spp., Streptococcus spp. Escherichia, Klebsiella, Proteus, Yersinia, Erwina, Salmonella, Listeria sp, Helicobacter pylori, mycobacteria (e.g. Mycobacterium tuberculosis) and Pseudomonas aeruginosa. For example, the bacterial infection may be sepsis or tuberculosis.

Phillips et al. Am J Pathol (2015) 185(3):820-833 describes upregulation of LAG-3 expression in the lungs and particularly in granulomatous lesions of rhesus macaques experimentally infected with Mycobacterium tuberculosis.

Examples of viral infections that may be treated include infection by influenza virus, measles virus, hepatitis B virus (HBV), hepatitis C virus (HCV), human immunodeficiency virus (HIV), lymphocytic choriomeningitis virus (LCMV), Herpes simplex virus and human papilloma virus.

Chronic viral infections, such as those caused by LCMV, HCV, HBV, and HIV commonly involve mechanisms to evade immune clearance. LAG-3 is expressed at high levels after LCMV infection in mice (Blackburn et al. Nat Immunol (2009) 10:29-37). Chen et al., J Gastroenterol Hepatol (2015) 30(12):1788-1795 describes negative regulation of the function of hepatitis C virus-specific CD8⁺ T cells in chronic hepatitis C patients, which can reversed by treatment with blocking anti-LAG-3 antibody. Li et al., Immunol Lett (2013) 150 (1-2): 116-122 describe a positive correlation between LAG-3 expression and HBV-specific CD8⁺ T cell dysfunction, and suggest a role for LAG-3 in the suppression of HBV-specific cell-mediated immunity in HCC. Tian et al. J Immunol 2015 194(8):3873-3782 describes association between upregulated LAG-3 expression on CD4⁺ and CD8⁺ T cells and disease progression in HIV infected patients.

Examples of fungal infections that may be treated include infection by Alternaria sp, Aspergillus sp, Candida sp and Histoplasma sp. The fungal infection may be fungal sepsis or histoplasmosis. The importance of T cell exhaustion in mediating fungal infection has been established e.g. by Chang et al. Critical Care (2013) 17:R85, and Lázár-Molnár et al PNAS (2008) 105(7): 2658-2663.

Examples of parasitic infections that may be treated include infection by Plasmodium species (e.g. Plasmodium falciparum, Plasmodium yoeli, Plasmodium ovale, Plasmodium vivax, or Plasmodium chabaudi chabaudi). The parasitic infection may be a disease such as malaria, leishmaniasis and toxoplasmosis.

Blockade of PD-L1 and LAG-3 using anti-PD-L1 and anti-LAG-3 monoclonal antibodies in vivo contributed to the restoration of CD4⁺ T-cell function, amplification of the number of follicular helper T cells, germinal-center B cells and plasmablasts, enhanced protective antibodies and rapidly cleared blood-stage malaria in mice. It was also shown to block the development of chronic infection (Butler et al., Nature Immunology Vol. 13, No. 12, p 188-195 February 2012).

Cancer

A cancer may be any unwanted cell proliferation (or any disease manifesting itself by unwanted cell proliferation), neoplasm or tumor or increased risk of or predisposition to the unwanted cell proliferation, neoplasm or tumor. The cancer may be benign or malignant and may be primary or secondary (metastatic). A neoplasm or tumor may be any abnormal growth or proliferation of cells and may be located in any tissue. Examples of tissues include the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, white blood cells.

Tumors to be treated may be nervous or non-nervous system tumors. Nervous system tumors may originate either in the central or peripheral nervous system, e.g. glioma, medulloblastoma, meningioma, neurofibroma, ependymoma, Schwannoma, neurofibrosarcoma, astrocytoma and oligodendroglioma. Non-nervous system cancers/tumors may originate in any other non-nervous tissue, examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T-cell lymphoma (CTCL), chronic lymphocytic leukemia (CLL), hepatoma, epidermoid carcinoma, prostate carcinoma, breast cancer, lung cancer, colon cancer, ovarian cancer, pancreatic cancer, thymic carcinoma, NSCLC, haematologic cancer and sarcoma.

Adoptive T Cell Transfer Therapy

In embodiments of the present invention, a method of treatment or prophylaxis may comprise adoptive cell transfer of immune cells. Adoptive T cell transfer therapy generally refers to a process in which white blood cells are removed from a subject, typically by drawing a blood sample from which white blood cells are separated, expanded in vitro or ex vivo and returned either to the same subject or to a different subject. The treatment is typically aimed at increasing the amount/concentration of an active form of the required T cell population in the subject. Such treatment may be beneficial in subjects experiencing T cell exhaustion.

Antibodies capable of blocking the mechanism of T cell exhaustion, or reversing it, provide a means of enhancing T cell activity and promoting T cell expansion.

Antibodies directed against immune checkpoint receptors (such as LAG-3) can also be useful in methods of T cell expansion, e.g. for expanding T cell populations of particular interest. For example, antibodies may be useful in methods of T cell expansion for preferentially expanding T cell subsets having desirable properties (e.g. in preference to T cell subsets having undesirable properties).

Accordingly, in a further aspect of the present invention a method is provided for expanding a population of T cells, wherein T cells are contacted in vitro or ex vivo with an antibody, antigen binding fragment, CAR, cell or polypeptide according to the present invention.

The method may optionally comprise one or more of the following steps: taking a blood sample from a subject; isolating T cells from the blood sample; culturing the T cells in in vitro or ex vivo cell culture (where they may be contacted with the antibody, antigen binding fragment, CAR, cell or polypeptide), collecting an expanded population of T cells; mixing the T cells with an adjuvant, diluent, or carrier; administering the expanded T cells to a subject.

Accordingly, in some aspects of the present invention a method of treatment of a subject having a T-cell dysfunctional disorder is provided, the method comprising obtaining a blood sample from a subject in need of treatment, culturing T cells obtained from the blood sample in the presence of an antibody, antigen binding fragment, CAR, cell or polypeptide according to the present invention so as to expand the T cell population, collecting expanded T cells, and administering the expanded T cells to a subject in need of treatment.

The T cells may be obtained from a subject requiring treatment, and may be isolated and/or purified. They may be a CD4⁺ and/or CD8⁺ T-cell population. The T-cells may represent a population experiencing T cell exhaustion and may optionally have upregulated expression of LAG-3.

During culture, T cells may be contacted with the antibody, antigen binding fragment, CAR, cell or polypeptide under conditions and for a period of time suitable to allow expansion of the T cells to a desired number of cells. After a suitable period of time the T cells may be harvested, optionally concentrated, and may be mixed with a suitable carrier, adjuvant or diluent and returned to the subject's body. A subject may undergo one or more rounds of such therapy.

Methods of T cell expansion are well known in the art, such as those described in Kalamasz et al., J Immunother 2004 September-October; 27(5):405-18; Montes et al., Clin Exp Immunol 2005 November; 142(2):292-302; Wölfl and Greenburg Nature Protocols 9 p 950-966 27 Mar. 2014; Trickett and Kwan Journal of Immunological Methods Vol. 275, Issues 1-2, 1 Apr. 2003, p 251-255; Butler et al PLoSONE 7(1) 12 Jan. 2012.

For example, methods of T cell expansion may comprise stimulating T cells. Stimulation may comprise non-specific stimulation, e.g. by treatment with anti-CD3/anti-CD28. Stimulation of T cells may comprise specific stimulation, e.g. by treatment with antigen (e.g. in complex with MHC, e.g. expressed by antigen presenting cells). Methods of T cell expansion may comprise culture in the presence of one or more factors for promoting T cell proliferation/expansion. For example, methods of T cell expansion may comprise culture in the presence of IL-2.

In the present invention, adoptive cell transfer (ACT) may be performed with the aim of introducing a cell or population of cells into a subject, and/or increasing the frequency of a cell or population of cells in a subject.

Adoptive transfer of T cells is described, for example, in Kalos and June 2013, Immunity 39(1): 49-60, which is hereby incorporated by reference in its entirety. Adoptive transfer of NK cells is described, for example, in Davis et al. 2015, Cancer J. 21(6): 486-491, which is hereby incorporated by reference in its entirety.

The cell may e.g. be a neutrophil, eosinophil, basophil, dendritic cell, lymphocyte, or monocyte. The lymphocyte may be e.g. a T cell, B cell, NK cell, NKT cell or innate lymphoid cell (ILC), or a precursor thereof. In some embodiments, the cell is a T cell. In some embodiments, the T cell is a CD3+ T cell. In some embodiments, the T cell is a CD3+, CD8+ T cell. In some embodiments, the T cell is a cytotoxic T cell (e.g. a cytotoxic T lymphocyte (CTL)). In some embodiments, the T cell is a virus-specific T cell. In some embodiments, the T cell is specific for EBV, HPV, HBV, HCV or HIV.

The present invention provides a method of treating or presenting a disease or condition in a subject, the method comprising modifying at least one cell obtained from a subject to express or comprise an antibody, antigen binding fragment, CAR, nucleic acid or vector according to the present invention, optionally expanding the modified at least one cell, and administering the modified at least one cell to a subject.

In some embodiments, the method comprises:
(a) isolating at least one cell from a subject;
(b) modifying the at least one cell to express or comprise an antibody, antigen binding fragment, CAR, nucleic acid or vector according to the present invention,
(c) optionally expanding the modified at least one cell, and;
(d) administering the modified at least one cell to a subject.

In some embodiments, the subject from which the cell is isolated is the subject administered with the modified cell (i.e., adoptive transfer is of autologous cells). In some embodiments, the subject from which the cell is isolated is a different subject to the subject to which the modified cell is administered (i.e., adoptive transfer is of allogenic cells).

The at least one cell modified according to the present invention can be modified according to methods well known to the skilled person. The modification may comprise nucleic acid transfer for permanent or transient expression of the transferred nucleic acid.

In some embodiments, the cell may additionally be modified to comprise or express a chimeric antigen receptor (CAR), or nucleic acid or vector encoding a CAR.

Any suitable genetic engineering platform may be used to modify a cell according to the present invention. Suitable methods for modifying a cell include the use of genetic engineering platforms such as gammaretroviral vectors, lentiviral vectors, adenovirus vectors, DNA transfection, transposon-based gene delivery and RNA transfection, for example as described in Maus et al., Annu Rev Immunol (2014) 32:189-225, incorporated by reference hereinabove.

In some embodiments the method may comprise one or more of the following steps: taking a blood sample from a subject; isolating and/or expanding at least one cell from the blood sample; culturing the at least one cell in in vitro or ex vivo cell culture; introducing into the at least one cell an antibody, antigen binding fragment, CAR, nucleic acid, or vector according to the present invention, thereby modifying the at least one cell; expanding the at least one modified cell; collecting the at least one modified cell; mixing the modified cell with an adjuvant, diluent, or carrier; administering the modified cell to a subject.

In some embodiments, the methods may additionally comprise treating the cell to induce/enhance expression of the antibody, antigen binding fragment, CAR, nucleic acid, or vector. For example, the nucleic acid/vector may comprise a control element for inducible upregulation of expression of the antibody, antigen binding fragment or CAR from the nucleic acid/vector in response to treatment with a particular agent. In some embodiments, treatment may be in vivo by administration of the agent to a subject having been administered with a modified cell according to the invention. In some embodiments, treatment may be ex vivo or in vitro by administration of the agent to cells in culture ex vivo or in vitro.

The skilled person is able to determine appropriate reagents and procedures for adoptive transfer of cells according to the present invention, for example by reference to Dai et al., 2016 J Nat Cancer Inst 108(7): djv439, which is incorporated by reference in its entirety. In a related aspect, the present invention provides a method of preparing a modified cell, the method comprising introducing into a cell a an antibody, antigen binding fragment, CAR, nucleic acid or vector according to the present invention, thereby modifying the at least one cell. The method is preferably performed in vitro or ex vivo.

In one aspect, the present invention provides a method of treating or preventing a disease or condition in a subject, comprising:
(a) isolating at least one cell from a subject;
(b) introducing into the at least one cell the nucleic acid or vector according to the present invention, thereby modifying the at least one cell; and
(c) administering the modified at least one cell to a subject.

In some embodiments, the cell may additionally be modified to introduce a nucleic acid or vector encoding a chimeric antigen receptor (CAR).

In some embodiments, the method additionally comprises therapeutic or prophylactic intervention, e.g. for the treatment or prevention of a cancer. In some embodiments, the therapeutic or prophylactic intervention is selected from chemotherapy, immunotherapy, radiotherapy, surgery, vaccination and/or hormone therapy.

Simultaneous or Sequential Administration

Compositions may be administered alone or in combination with other treatments, either simultaneously or sequentially dependent upon the condition to be treated.

In this specification an antibody, antigen binding fragment, CAR, cell or polypeptide of the present invention and an anti-infective agent or chemotherapeutic agent (therapeutic agent) may be administered simultaneously or sequentially.

In some embodiments, treatment with an antibody, antigen binding fragment, CAR, cell or polypeptide of the present invention may be accompanied by chemotherapy.

Simultaneous administration refers to administration of the antibody, antigen binding fragment, CAR, cell or polypeptide and therapeutic agent together, for example as a pharmaceutical composition containing both agents (combined preparation), or immediately after each other and optionally via the same route of administration, e.g. to the same artery, vein or other blood vessel.

Sequential administration refers to administration of one of the antibody, antigen binding fragment, CAR, cell or polypeptide or therapeutic agent followed after a given time interval by separate administration of the other agent. It is not required that the two agents are administered by the same route, although this is the case in some embodiments. The time interval may be any time interval.

Combined inhibition of the PD-1/PD-L1 pathway and LAG-3 blockade has been shown to provide anti-tumour efficacy (Jing et al. Journal for ImmunoTherapy of Cancer (2015) 3:2; also Nguyen and Ohashi, Nat Rev Immunol (2015) 15:45-56). Accordingly, in one aspect the present invention provides the antibody, antigen binding fragment, CAR, cell or polypeptide according to the present invention for use in a combination therapy with an inhibitor of the PD-1/PD-L1 pathway.

In some embodiments, the present invention provides combination therapy with an inhibitor of PD-1, PD-L1 or the PD-1/PD-L1 pathway. In some embodiments, the inhibitor is an agent capable of inhibiting or preventing signalling mediated by interaction between PD-1 and PD-L1. In some embodiments, the inhibitor is an agent capable of downregulating gene or protein expression of PD-1 and/or PD-L1. In some embodiments, the inhibitor is an agent capable of inhibiting or preventing binding between PD-1 and PD-L1. In some embodiments, the agent is an antibody. In some embodiments, the agent is an antibody capable of binding to PD-1. In some embodiments, the agent is an antibody capable of binding to PD-L1. The antibody may be an antagonist antibody, or a blocking antibody. Inhibitors of PD-1, PD-L1 or the PD-1/PD-L1 pathway are well known to the skilled person, and include, for example, nivolumab, pidilizumab, BMS 936559, MPDL328oA, pembrolizumab, and avelumab. PD-1/PDL-1 inhibitors contemplated for use in accordance with the present invention include those described in Sunshine and Taube “PD-1/PD-L1 inhibitors”, Curr. Opin. Pharmacol. 2015, 23:32-38, which is hereby incorporated by reference in its entirety.

Anti-Infective Agents

In treating infection, an antibody, antigen binding fragment, CAR, cell or polypeptide of the present invention may be administered in combination with an anti-infective agent, as described above. The anti-infective agent may be an agent known to have action against the microorganism or virus responsible for the infection.

Suitable anti-infective agents include antibiotics (such as penicillins, cephalosporins, rifamycins, lipiarmycins, quinolones, sulfonamides, macrolides, lincosamides, tetracyclines, cyclic lipopeptides, glycylcyclines, oxazolidinones, and lipiarmycins), anti-viral agents (such as reverse transcriptase inhibitors, integrase inhibitors, transcription factor inhibitors, antisense and siRNA agents and protease inhibitors), anti-fungal agents (such as polyenes, imidiazoles, triazoles, thiazoles, allylamines, and echinocandins) and anti-parasitic agents (such as antinematode agents, anticestode agents, antitrematode agents, antiamoebic agents and antiprotozoal agents).

Chemotherapy

Chemotherapy refers to treatment of a cancer with a drug or with ionising radiation (e.g. radiotherapy using X-rays or γ-rays). In preferred embodiments chemotherapy refers to treatment with a drug. The drug may be a chemical entity, e.g. small molecule pharmaceutical, antibiotic, DNA intercalator, protein inhibitor (e.g. kinase inhibitor), or a biological agent, e.g. antibody, antibody fragment, nucleic acid or peptide aptamer, nucleic acid (e.g. DNA, RNA), peptide, polypeptide, or protein. The drug may be formulated as a pharmaceutical composition or medicament. The formulation may comprise one or more drugs (e.g. one or more active agents) together with one or more pharmaceutically acceptable diluents, excipients or carriers.

A treatment may involve administration of more than one drug. A drug may be administered alone or in combination with other treatments, either simultaneously or sequentially dependent upon the condition to be treated. For example, the chemotherapy may be a co-therapy involving administration of two drugs, one or more of which may be intended to treat the cancer.

The chemotherapy may be administered by one or more routes of administration, e.g. parenteral, intravenous injection, oral, subcutaneous, intradermal or intratumoral.

The chemotherapy may be administered according to a treatment regime. The treatment regime may be a pre-determined timetable, plan, scheme or schedule of chemotherapy administration which may be prepared by a physician or medical practitioner and may be tailored to suit the patient requiring treatment.

The treatment regime may indicate one or more of: the type of chemotherapy to administer to the patient; the dose of each drug or radiation; the time interval between administrations; the length of each treatment; the number and nature of any treatment holidays, if any etc. For a co-therapy a single treatment regime may be provided which indicates how each drug is to be administered.

Chemotherapeutic drugs and biologics may be selected from: alkylating agents such as cisplatin, carboplatin, mechlorethamine, cyclophosphamide, chlorambucil, ifosfamide; purine or pyrimidine anti-metabolites such as azathiopurine or mercaptopurine; alkaloids and terpenoids, such as vinca alkaloids (e.g. vincristine, vinblastine, vinorelbine, vindesine), podophyllotoxin, etoposide, teniposide, taxanes such as paclitaxel (Taxol™), docetaxel; topoisomerase inhibitors such as the type I topoisomerase inhibitors camptothecins irinotecan and topotecan, or the type II topoisomerase inhibitors amsacrine, etoposide, etoposide phosphate, teniposide; antitumor antibiotics (e.g. anthracyline antibiotics) such as dactinomycin, doxorubicin (Adriamycin™), epirubicin, bleomycin, rapamycin; antibody based agents, such as anti-PD-1 antibodies, anti-PD-L1 antibodies, anti-TIM-3 antibodies, anti-CTLA-4, anti-4-1BB, anti-GITR, anti-CD27, anti-BLTA, anti-OX43, anti-VEGF, anti-TNFα, anti-IL-2, antiGpIIb/IIIa, anti-CD-52, anti-CD20, anti-RSV, anti-HER2/neu(erbB2), anti-TNF receptor, anti-EGFR antibodies, monoclonal antibodies or antibody fragments, examples include: cetuximab, panitumumab, infliximab, basiliximab, bevacizumab (Avastin®), abciximab, daclizumab, gemtuzumab, alemtuzumab, rituximab (Mabthera®), palivizumab, trastuzumab, etanercept, adalimumab, nimotuzumab; EGFR inihibitors such as erlotinib, cetuximab and gefitinib; anti-angiogenic agents such as bevacizumab (Avastin®); cancer vaccines such as Sipuleucel-T (Provenge®).

In one embodiment the chemotherapeutic agent is an anti-PD-1 antibody, anti-PD-L1 antibody, anti-TIM-3 antibody, anti-CTLA-4, anti-41 BB, anti-GITR, anti-CD27, anti-BLTA, anti-OX43, anti-VEGF, anti-TNFα, anti-IL2, anti-GpIIb/IIIa, anti-CD-52, anti-CD20, anti-RSV, anti-HER2/neu(erbB2), anti-TNF receptor, anti-EGFR or other antibody. In some embodiments, the chemotherapeutic agent is an immune checkpoint inhibitor or costimulation molecule.

Further chemotherapeutic drugs may be selected from: 13-cis-Retinoic Acid, 2-Chlorodeoxyadenosine, 5-Azacitidine 5-Fluorouracil, 6-Mercaptopurine, 6-Thioguanine, Abraxane, Accutane®, Actinomycin-D Adriamycin®, Adrucil®, Afinitore, Agryline, Ala-Corte, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Aranesp®, Aredia®, Arimidex®, Aromasin®, Arranon®, Arsenic Trioxide, Asparaginase, ATRA Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, Calcium Leucovorin, Campath®, Camptosar®, Camptothecin-11, Capecitabine, Carac™′ Carboplatin, Carmustine, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine Cytosar-U®, Cytoxan®, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin, Diftitox, DepoCyt™, Dexamethasone, Dexamethasone Acetate, Dexamethasone Sodium Phosphate, Dexasone, Dexrazoxane, DHAD, DIC, Diodex, Docetaxel, Doxil®, Doxorubicin, Doxorubicin Liposomal, Droxia™ DTIC, DTIC-Dome®, Duralone®, Eligard™, Ellence™, Eloxatin™, Elspar®, Emcyt®, Epirubicin, Epoetin Alfa, Erbitux, Erlotinib, Erwinia L-asparaginase, Estramustine, Ethyol Etopophos®, Etoposide, Etoposide Phosphate, Eulexin®, Everolimus, Evista®, Exemestane, Faslodex®, Ferrara®, Filgrastim, Floxuridine, Fludara®, Fludarabine, Fluoroplex®, Fluorouracil, Fluoxymesterone, Flutamide, Folinic Acid, FUDR®, Fulvestrant, Gefitinib, Gemcitabine, Gemtuzumab ozogamicin, Gleevec™, Gliadel® Wafer, Goserelin, Granulocyte—Colony Stimulating Factor, Granulocyte Macrophage Colony Stimulating Factor, Herceptin®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab Tiuxetan, Idamycin®, Idarubicin, Ifex®, IFN-alpha, Ifosfamide, IL-11, IL-2, Imatinib mesylate, Imidazole Carboxamide, Interferon alfa, Interferon Alfa-2b (PEG Conjugate), Interleukin-2, Interleukin-11, Intron A® (interferon alfa-2b), Iressa®, Irinotecan, Isotretinoin, Ixabepilone, Ixempra™, Kidrolase, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mylocel™, Mylotarg®, Navelbine®, Nelarabine, Neosar®, Neulasta™, Neumega®, Neupogen®, Nexavar®, Nilandron®, Nilutamide, Nipent®, Nitrogen Mustard, Novaldex®, Novantrone®, Octreotide, Octreotide acetate, Oncospar®, Oncovin®, Ontak®, Onxal™, Oprevelkin, Orapred®, Orasone®, Oxaliplatin, Paclitaxel, Paclitaxel Protein-bound, Pamidronate, Panitumumab, Panretin®, Paraplatin®, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant Purinethol®, Raloxifene, Revlimid®, Rheumatrex®, Rituxan®, Rituximab, Roferon-A® (Interferon Alfa-2a), Rubex®, Rubidomycin hydrochloride, Sandostatin® Sandostatin LAR®, Sargramostim, Solu-Cortef®, Solu-Medrol®, Sorafenib, SPRYCEL™, STI-571, Streptozocin, SU11248, Sunitinib, Sutent®, Tamoxifen, Tarceva®, Targretin®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, VP-16, Vumon®, Xeloda®, Zanosar®, Zevalin™, Zinecard®, Zoladex®, Zoledronic acid, Zolinza, Zometa®.

Routes of Administration

Antibodies, antigen binding fragments, CARs, cells, polypeptides and other therapeutic agents, medicaments and pharmaceutical compositions according to aspects of the present invention may be formulated for administration by a number of routes, including but not limited to, parenteral, intravenous, intra-arterial, intramuscular, subcutaneous, intradermal, intratumoral and oral. Antibodies, antigen binding fragments, CARs, cells, polypeptides and other therapeutic agents, may be formulated in fluid or solid form. Fluid formulations may be formulated for administration by injection to a selected region of the human or animal body.

Dosage Regime

Multiple doses of the antibody, antigen binding fragment, CAR, cell or polypeptide may be provided. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

Kits

In some aspects of the present invention a kit of parts is provided. In some embodiments the kit may have at least one container having a predetermined quantity of the antibody, antigen binding fragment, CAR, cell or polypeptide. The kit may provide the antibody, antigen binding fragment, CAR, cell or polypeptide in the form of a medicament or pharmaceutical composition, and may be provided together with instructions for administration to a patient in order to treat a specified disease or condition. The antibody, antigen binding fragment, CAR, cell or polypeptide may be formulated so as to be suitable for injection or infusion to a tumor or to the blood.

In some embodiments the kit may further comprise at least one container having a predetermined quantity of another therapeutic agent (e.g. anti-infective agent or chemotherapy agent). In such embodiments, the kit may also comprise a second medicament or pharmaceutical composition such that the two medicaments or pharmaceutical compositions may be administered simultaneously or separately such that they provide a combined treatment for the specific disease or condition. The therapeutic agent may also be formulated so as to be suitable for injection or infusion to a tumor or to the blood.

Subjects

The subject to be treated may be any animal or human. The subject is preferably mammalian, more preferably human. The subject may be a non-human mammal, but is more preferably human. The subject may be male or female. The subject may be a patient. A subject may have been diagnosed with a disease or condition requiring treatment, or be suspected of having such a disease or condition.

Protein Expression

Molecular biology techniques suitable for producing polypeptides according to the invention in cells are well known in the art, such as those set out in Sambrook et al., Molecular Cloning: A Laboratory Manual, New York: Cold Spring Harbor Press, 1989

The polypeptide may be expressed from a nucleotide sequence. The nucleotide sequence may be contained in a vector present in a cell, or may be incorporated into the genome of the cell.

A “vector” as used herein is an oligonucleotide molecule (DNA or RNA) used as a vehicle to transfer exogenous genetic material into a cell. The vector may be an expression vector for expression of the genetic material in the cell. Such vectors may include a promoter sequence operably linked to the nucleotide sequence encoding the gene sequence to be expressed. A vector may also include a termination codon and expression enhancers. Any suitable vectors, promoters, enhancers and termination codons known in the art may be used to express polypeptides from a vector according to the invention. Suitable vectors include plasmids, binary vectors, viral vectors and artificial chromosomes (e.g. yeast artificial chromosomes).

In this specification the term “operably linked” may include the situation where a selected nucleotide sequence and regulatory nucleotide sequence (e.g. promoter and/or enhancer) are covalently linked in such a way as to place the expression of the nucleotide sequence under the influence or control of the regulatory sequence (thereby forming an expression cassette). Thus a regulatory sequence is operably linked to the selected nucleotide sequence if the regulatory sequence is capable of effecting transcription of the nucleotide sequence. Where appropriate, the resulting transcript may then be translated into a desired protein or polypeptide.

Any cell suitable for the expression of polypeptides may be used for producing peptides according to the invention. The cell may be a prokaryote or eukaryote. Suitable prokaryotic cells include E. coli. Examples of eukaryotic cells include a yeast cell, a plant cell, insect cell or a mammalian cell. In some cases the cell is not a prokaryotic cell because some prokaryotic cells do not allow for the same post-translational modifications as eukaryotes. In addition, very high expression levels are possible in eukaryotes and proteins can be easier to purify from eukaryotes using appropriate tags. Specific plasmids may also be utilised which enhance secretion of the protein into the media.

Methods of producing a polypeptide of interest may involve culture or fermentation of a cell modified to express the polypeptide. The culture or fermentation may be performed in a bioreactor provided with an appropriate supply of nutrients, air/oxygen and/or growth factors. Secreted proteins can be collected by partitioning culture media/fermentation broth from the cells, extracting the protein content, and separating individual proteins to isolate secreted polypeptide. Culture, fermentation and separation techniques are well known to those of skill in the art.

Bioreactors include one or more vessels in which cells may be cultured. Culture in the bioreactor may occur continuously, with a continuous flow of reactants into, and a continuous flow of cultured cells from, the reactor. Alternatively, the culture may occur in batches. The bioreactor monitors and controls environmental conditions such as pH, oxygen, flow rates into and out of, and agitation within the vessel such that optimum conditions are provided for the cells being cultured.

Following culture of cells that express the polypeptide of interest, that polypeptide is preferably isolated. Any suitable method for separating polypeptides/proteins from cell culture known in the art may be used. In order to isolate a polypeptide/protein of interest from a culture, it may be necessary to first separate the cultured cells from media containing the polypeptide/protein of interest. If the polypeptide/protein of interest is secreted from the cells, the cells may be separated from the culture media that contains the secreted polypeptide/protein by centrifugation. If the polypeptide/protein of interest collects within the cell, it will be necessary to disrupt the cells prior to centrifugation, for example using sonification, rapid freeze-thaw or osmotic lysis. Centrifugation will produce a pellet containing the cultured cells, or cell debris of the cultured cells, and a supernatant containing culture medium and the polypeptide/protein of interest.

It may then be desirable to isolate the polypeptide/protein of interest from the supernatant or culture medium, which may contain other protein and non-protein components. A common approach to separating polypeptide/protein components from a supernatant or culture medium is by precipitation. Polypeptides/proteins of different solubility are precipitated at different concentrations of precipitating agent such as ammonium sulfate. For example, at low concentrations of precipitating agent, water soluble proteins are extracted. Thus, by adding increasing concentrations of precipitating agent, proteins of different solubility may be distinguished. Dialysis may be subsequently used to remove ammonium sulfate from the separated proteins.

Other methods for distinguishing different polypeptides/proteins are known in the art, for example ion exchange chromatography and size chromatography. These may be used as an alternative to precipitation, or may be performed subsequently to precipitation.

Once the polypeptide/protein of interest has been isolated from culture it may be necessary to concentrate the protein. A number of methods for concentrating a protein of interest are known in the art, such as ultrafiltration or lyophilisation.

Sequence Identity

Alignment for purposes of determining percent amino acid or nucleotide sequence identity can be achieved in various ways known to a person of skill in the art, for instance, using publicly available computer software such as ClustalW 1.82. T-coffee or Megalign (DNASTAR) software. When using such software, the default parameters, e.g. for gap penalty and extension penalty, are preferably used. The default parameters of ClustalW 1.82 are: Protein Gap Open Penalty=10.0, Protein Gap Extension Penalty=0.2, Protein matrix=Gonnet, Protein/DNA ENDGAP=−1, Protein/DNA GAPDIST=4.

The invention includes the combination of the aspects and preferred features described except where such a combination is clearly impermissible or expressly avoided.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

### BRIEF DESCRIPTION OF THE FIGURES

Embodiments and experiments illustrating the principles of the invention will now be discussed with reference to the accompanying figures in which:

FIG. 1. Light chain variable domain sequences for anti-LAG-3 antibody clones A6, 1G11, C2, C12, F5 and G8. CDRs are underlined and shown separately.

FIG. 2. Heavy chain variable domain sequences for anti-LAG-3 antibody clones A6, 1G11, C2, C12, F5 and G8. CDRs are underlined and shown separately.

FIG. 3. Table showing light chain and heavy chain CDR sequences for anti-LAG-3 antibody clones A6, 1G11, C2, C12, F5 and G8.

FIG. 4. Nucleotide and encoded amino acid sequences of heavy and light chain variable domain sequences for anti-LAG-3 antibody clones A6, 1G11, C2, C12, F5 and G8.

FIG. 5. Bar chart showing binding of anti-LAG-3 antibodies to the Fc-coupled human and murine LAG-3, as determined by ELISA.

FIG. 6. Bar chart showing binding of anti-LAG-3 antibodies to the Fc-coupled human and murine LAG-3, as determined by ELISA.

FIG. 7. Graph showing binding of A6, F5 and G8 antibodies in IgG1 or IgG4 format to human LAG-3, as determined by ELISA. Mean±SD of triplicates is shown.

FIG. 8. Bar chart showing binding of A6, F5 and G8 antibodies in IgG1 format to human LAG-3-transfected HEK293 cells, or untransfected, PBS-treated control cells. Geometric mean fluorescence intensities (MFIs) are shown.

FIG. 9. Bar chart showing binding of A6, F5 and G8 antibodies in IgG1 format to activated CD4⁺ T cells, or unactivated control CD4⁺ T cells. Geometric MFIs are shown.

FIG. 10. Bar chart showing binding of A6, F5 and G8 antibodies in IgG1 format to rhesus macaque LAG-3-transfected HEK293 cells, or untransfected control cells.

FIGS. 11A-C. Sensorgrams and Table showing binding of A6 Fab to immobilised, Fc-coupled human or murine LAG-3, as determined by Surface Plasmon Resonance.

(FIG. 11A) Sensorgram showing binding of A6 Fab to human LAG-3. (FIG. 11B) Sensorgram showing binding of A6 Fab to murine LAG-3. (FIG. 11C) Table showing affinity of A6 Fab for human LAG-3.

FIG. 12. Table showing affinity of antibodies A6, F5, G8 and BMS-986016 to human LAG-3 as determined by Bio-Layer Interferometry.

FIG. 13. Graph showing inhibition of LAG-3 binding to MHC class II on Daudi cells by A6 and 1G11 Fab.

FIGS. 14A-B. Graph and Table showing inhibition of LAG-3 binding to MHC class II on Daudi cells. (FIG. 14A) Graph showing inhibition of LAG-3 binding to MHC class II on Daudi cells by A6, C2, C12, F5 and G8. (FIG. 14B) Table showing IC₅₀ values for inhibition of LAG-3 binding to MHC class II by A6, C2, C12, F5 and G8.

FIG. 15. Graph showing inhibition of LAG-3 binding to MHC class II on Daudi cells by A6, C2, C12, F5 and G8.

FIGS. 16A-B. Bar charts showing IL-2 production in MLR assay following treatment with F5 or G8 antibody in IgG1 format, or IgG1 isotype control. (FIG. 16A) and (FIG. 16B) show the results of two independent experiments. Mean±SD of triplicates is shown. The line indicates maximum mean background detected in the presence of the isotype control.

FIGS. 17A-B. Bar charts showing IFN-γ production in MLR assay following treatment with F5 or G8 antibody in IgG1 format, or IgG1 isotype control. (FIG. 17A) and (FIG. 17B) show the results of two independent experiments. Mean±SD of triplicates is shown. The line indicates maximum mean background detected in the presence of the isotype control.

FIGS. 18A-D. Graphs showing Bio-Layer Interferometry analysis of epitopes for anti-LAG-3 antibodies. Binding profiles of the indicated antibodies to LAG-3 bound to (FIG. 18A) BMS-986016, (FIG. 18B) A6, (FIG. 18C) F5, and (FIG. 18D) G8 are shown.

FIG. 19. Graph showing the number of T cells following expansion by culture with anti-CD3/CD28 beads in the presence of IL-2, in the absence of anti-LAG-3 antibody (clone F5, IgG1) or in the presence of different amounts of the anti-LAG-3 antibody. Cell number counts were normalised to a ‘CD3/CD28 beads only’ control condition.

FIGS. 20A-C. Graphs showing the numbers of (FIG. 20A) CD8+ T cells and (FIG. 20B) CD4+ T cells following expansion by culture with anti-CD3/CD28 beads in the presence of IL-2, in the absence of anti-LAG-3 antibody (clone F5, IgG1) or in the presence of different amounts of the anti-LAG-3 antibody, and (FIG. 20C) showing the ratio of CD8:CD4 cells. Cell number counts were normalised to a ‘CD3/CD28 beads only’ control condition.

FIG. 21. Graph showing the percentage of CD4+CD25+FoxP3+ Tregs within the CD4+ T cell population following expansion by culture with anti-CD3/CD28 beads in the presence of IL-2, in the absence of anti-LAG-3 antibody (clone F5, IgG1) or in the presence of different amounts of the anti-LAG-3 antibody, normalised to a ‘CD3/CD28 beads only’ control condition.

FIGS. 22A-B. Graphs showing (FIG. 22A) the percentage of CD8+PD1+ cells within the CD8+ T cell population, and (FIG. 22B) the percentage of CD4+PD1+ cells within the CD4+ T cell population following expansion by culture with anti-CD3/CD28 beads in the presence of IL-2, in the absence of anti-LAG-3 antibody (clone F5, IgG1) or in the presence of different amounts of the anti-LAG-3 antibody, normalised to a ‘CD3/CD28 beads only’ control condition.

FIGS. 23A-B. Bar charts showing percentages of different T cell subpopulations within the (FIG. 23A) CD8+ T cell population and (FIG. 23B) CD4+ T cell population following expansion by culture with anti-CD3/CD28 beads in the presence of IL-2, in the absence of anti-LAG-3 antibody (clone F5, IgG1) or in the presence of different amounts of the anti-LAG-3 antibody, normalised to a ‘CD3/CD28 beads only’ control condition.

FIGS. 24A-B. Graphs showing (FIG. 24A) the percentage of CD8+CTLA4+ cells within the CD8+ T cell population, and (FIG. 24B) the percentage of CD4+CTLA4+ cells within the CD4+ T cell population following expansion by culture with anti-CD3/CD28 beads in the presence of IL-2, in the absence of anti-LAG-3 antibody (clone F5, IgG1) or in the presence of different amounts of the anti-LAG-3 antibody, normalised to a ‘CD3/CD28 beads only’ control condition.

FIGS. 25A-B. Graphs showing (FIG. 25A) the percentage of CD8+IL-13+ cells within the CD8+ T cell population, and (FIG. 25B) the percentage of CD4+IL-13+ cells within the CD4+ T cell population following expansion by culture with anti-CD3/CD28 beads in the presence of IL-2, in the absence of anti-LAG-3 antibody (clone F5, IgG1) or in the presence of different amounts of the anti-LAG-3 antibody, normalised to a ‘CD3/CD28 beads only’ control condition.

FIGS. 26A-B. Graphs showing (FIG. 26A) the percentage of CD8+IFNγ+ cells within the CD8+ T cell population, and (FIG. 26B) the percentage of CD4+IFNγ+ cells within the CD4+ T cell population following expansion by culture with anti-CD3/CD28 beads in the presence of IL-2, in the absence of anti-LAG-3 antibody (clone F5, IgG1) or in the presence of different amounts of the anti-LAG-3 antibody, normalised to a ‘CD3/CD28 beads only’ control condition.

FIGS. 27A-B. Graphs showing (FIG. 27A) the percentage of CD8+TNFα+ cells within the CD8+ T cell population, and (FIG. 27B) the percentage of CD4+TNFα+ cells within the CD4+ T cell population following expansion by culture with anti-CD3/CD28 beads in the presence of IL-2, in the absence of anti-LAG-3 antibody (clone F5, IgG1) or in the presence of different amounts of the anti-LAG-3 antibody, normalised to a ‘CD3/CD28 beads only’ control condition.

FIGS. 28A-B. Graphs showing (FIG. 28A) the percentage of CD56+ cells, and (FIG. 28B) the percentage CD19+ cells within the expanded population of cells following expansion by culture with anti-CD3/CD28 beads in the presence of IL-2, in the absence of anti-LAG-3 antibody (clone F5, IgG1) or in the presence of different amounts of the anti-LAG-3 antibody, normalised to a ‘CD3/CD28 beads only’ control condition.

### EXAMPLES

The inventors describe in the following Examples isolation and characterisation of several anti-human LAG-3 antibodies, which are shown to specifically bind to human LAG-3 and to block the engagement of LAG-3 to MHC class II, thereby inhibiting LAG-3 signaling.

### Example 1: Isolation of Anti-Human LAG-3 Antibodies, and Binding to Human and Murine LAG-3

Anti-LAG-3 antibodies were isolated from a human antibody phage display library via in vitro selection in a 3-round bio-panning process.

Human LAG-3 coupled to human Fc (LAG-3-Fc) was biotinylated and coated onto streptavidin-magnetic beads. The coated beads were used to isolate anti-LAG-3-specific phages using magnetic sorting. Some steps to get rid of potential anti-biotin and anti-human Fc antibodies were added in the selection process.

After a small-scale induction in HB2151 cells, Fab antibodies were screened by ELISA for ability to bind to human and murine LAG-3. Briefly, ELISA plates were coated with human LAG-3-Fc and blocked with a solution of casein. After extensive washes in PBS Tween-20, crude periplasmic extracts from the induction were transferred into the ELISA wells in the presence of 7% milk in PBS. After 90 minutes at room temperature under agitation and extensive washes, a goat anti-human Fab antibody coupled to HRP was added. One hour la ter, plates were washed and TMB substrate was added. The reaction was stopped with 1M HCl and optical density was measured at 450 nm with a reference at 670 nm. Antibodies giving an absorbance >0.1 were selected as positive. A first clonality screening was performed by DNA fingerprinting; clonality was then confirmed by sequencing.

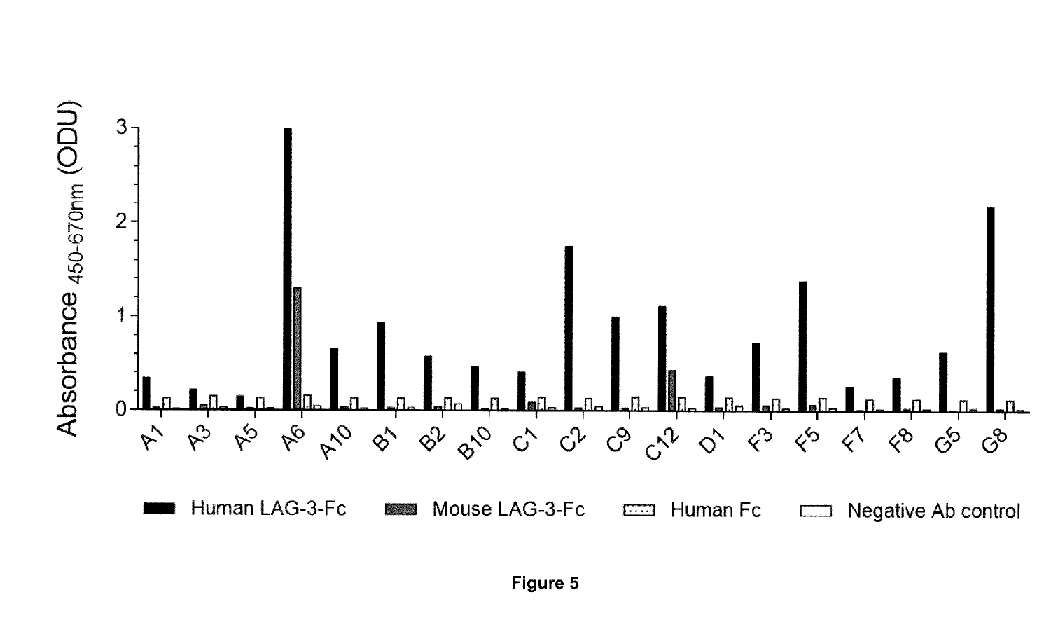

Nineteen unique clones showing a positive binding to human LAG-3 in ELISA were selected (FIG. 5). Amongst these, 2 clones showed both high binding to human LAG-3 and cross-reactivity to mouse LAG-3: A6 and C12.

### Example 2: Isolation of Anti-Murine LAG-3 Antibodies, and Binding to Human and Murine LAG-3

Anti-mouse LAG-3 antibodies were isolated from the phage library by the same selection process as described in Example 1, using mouse LAG-3 coupled to human Fc.

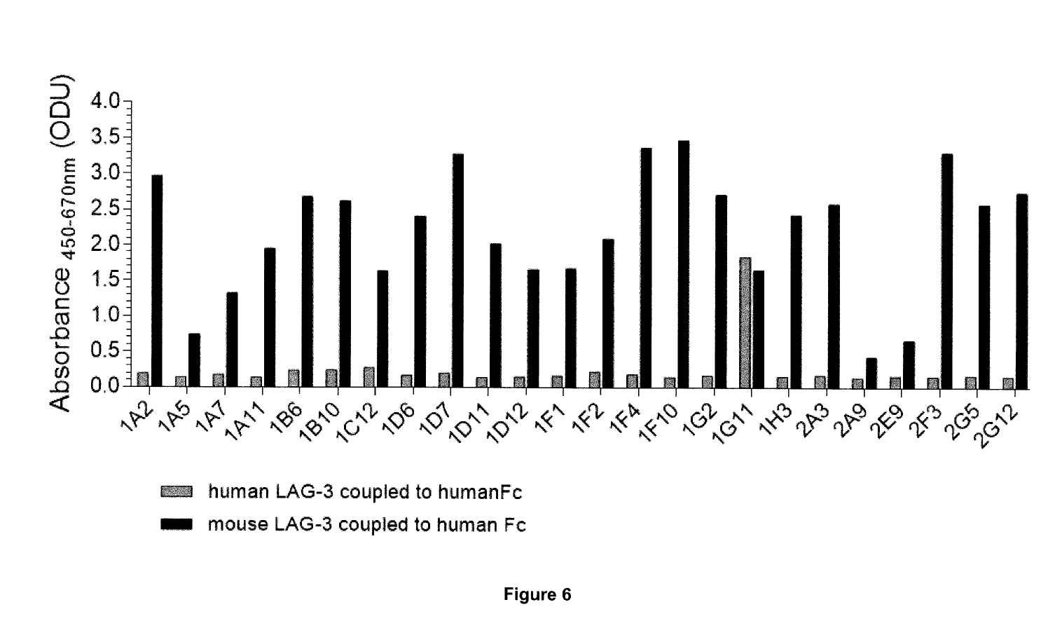

Various clones showing a positive binding to murine LAG-3 in ELISA were identified, all but one were specific to mouse LAG-3 and did not recognise human LAG-3 (FIG. 6). Clone 1G11 showed similar binding to human and mouse LAG-3.

### Example 3: Binding of A6, F5, and G8 Antibodies to Soluble Recombinant Human LAG-3 Protein

Binding of anti-LAG-3 antibodies, either in IgG1 or IgG4 format, was assessed by ELISA. Antibodies were coated on the ELISA plate and biotinylated recombinant human LAG-3 was added before revelation using streptavidin.

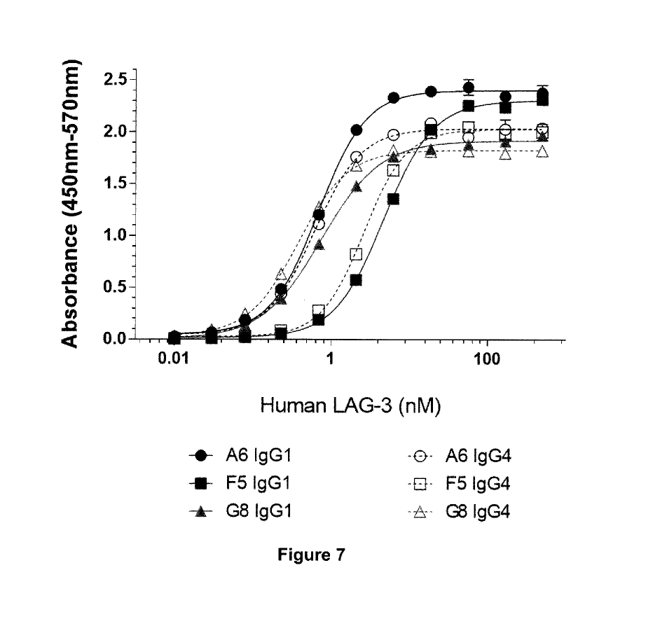

FIG. 7 shows the binding of A6, F5 and G8 antibody clones (mean±SD on duplicates). All antibodies were shown to bind to LAG-3 in a dose-dependent manner. A6 and G8 displayed higher affinity for human LAG-3 than F5. The isotype IgG1 or IgG4 did not appear to alter the binding of the clones to human LAG-3.

### Example 4: Binding of A6, F5, and G8 Antibodies to Transiently Transfected Cells Expressing Human LAG-3

The ability of A6, F5, and G8 antibodies to bind LAG-3 expressed at the surface of cells was evaluated. Briefly, HEK-293 cells were transiently transfected with human LAG-3 and antibody binding was measured at day 2 by flow cytometry.

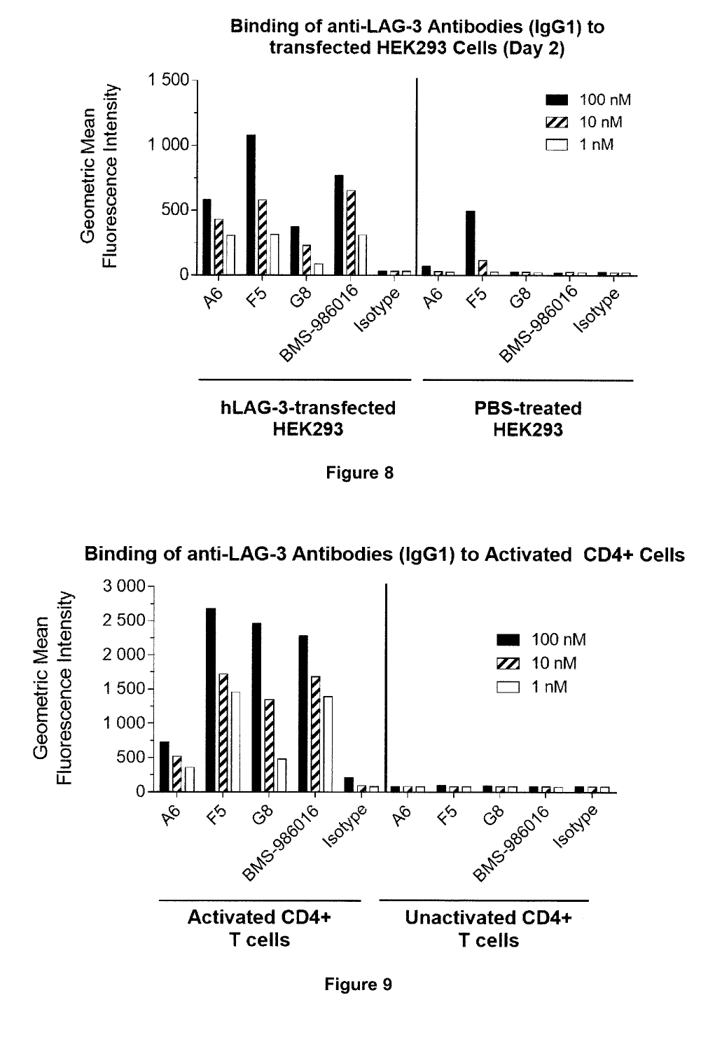

FIG. 8 shows binding of the antibodies to LAG-3-transfected cells or untransfected PBS-treated control cells (geometric mean fluorescence intensities (MFIs) are shown). Anti-LAG-3 antibodies A6, F5 and G8 were shown to bind to the cell surface of LAG-3 expressing cells to a similar extent as reference anti-LAG-3 antibody BMS-986016. F5 showed a higher binding affinity for LAG-3 than other antibodies, but also displayed some non-specific binding to untransfected cells.

### Example 5: Binding of A6, F5, and G8 Antibodies to Activated T Cells

Binding of A6, F5, and G8 antibodies to activated T cells was assessed. Briefly, CD4⁺ cells were isolated from PBMC samples and stimulated with anti-CD3/CD28 beads for 3 days. The anti-LAG-3 antibodies were then added onto cells and binding was measured by flow cytometry.

FIG. 9 shows binding of the anti-LAG-3 antibodies to activated and unactivated T cells (geometric mean fluorescence intensities (MFIs) are shown). F5 and G8 show high binding to activated T cells, similar to the extent of binding for reference anti-LAG-3 antibody BMS-986016. A6 exhibited an intermediate level of binding. None of the tested antibodies show non-specific binding to non-activated T cells.

### Example 6: Binding of A6, F5, and G8 Antibodies to Rhesus LAG-3

The ability of A6, F5, and G8 antibodies to bind to rhesus macaque LAG-3 was tested using transiently transfected HEK-293 cells.

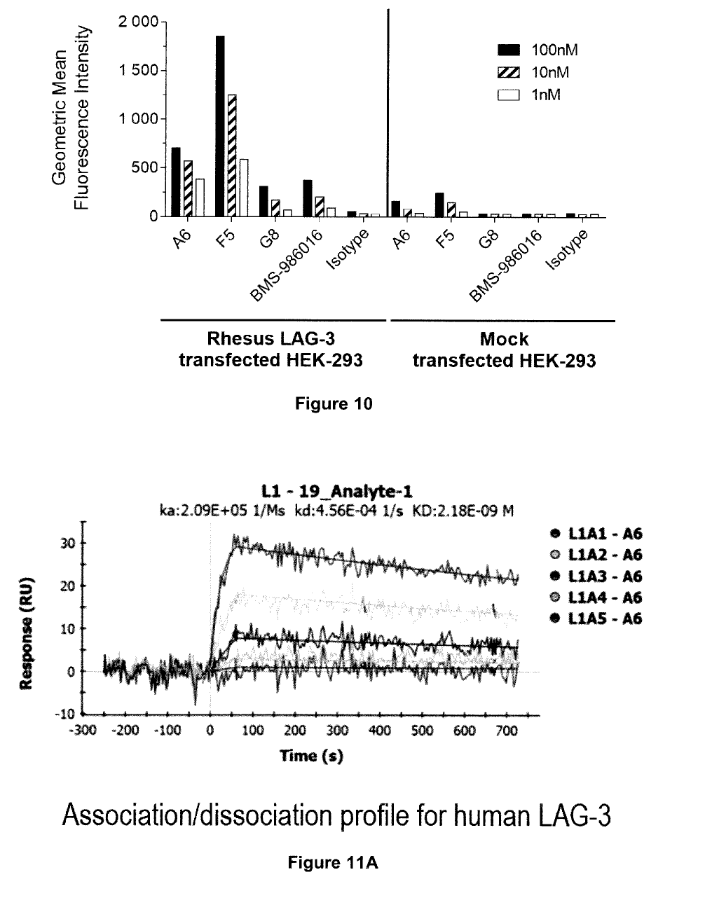

FIG. 10 shows the binding of anti-LAG-3 antibodies to cells expressing rhesus LAG-3 and to untransfected negative control cells. All of A6, F5, and G8 antibodies were shown to bind to rhesus LAG-3. Binding of A6 and F5 to rhesus LAG-3 was high, whilst binding by G8 was weaker. The level of binding of G8 to rhesus LAG-3 was similar to binding of reference anti-LAG-3 antibody BMS-986016. A6 and F5 displayed a small degree of unspecific background binding to untransfected cells.

### Example 7: Affinity of Binding to LAG-3

The affinity of antibody clone A6 Fab was measured by Surface Plasmon Resonance analysis. Briefly, human or mouse LAG-3 coupled to human Fc was immobilised on a sensor chip and the antibody was applied onto the chip at different concentrations. Association and dissociation rates were measured with a ProteOn XPR 36 analyser (Biorad) and the affinity (K_{D}) was calculated.

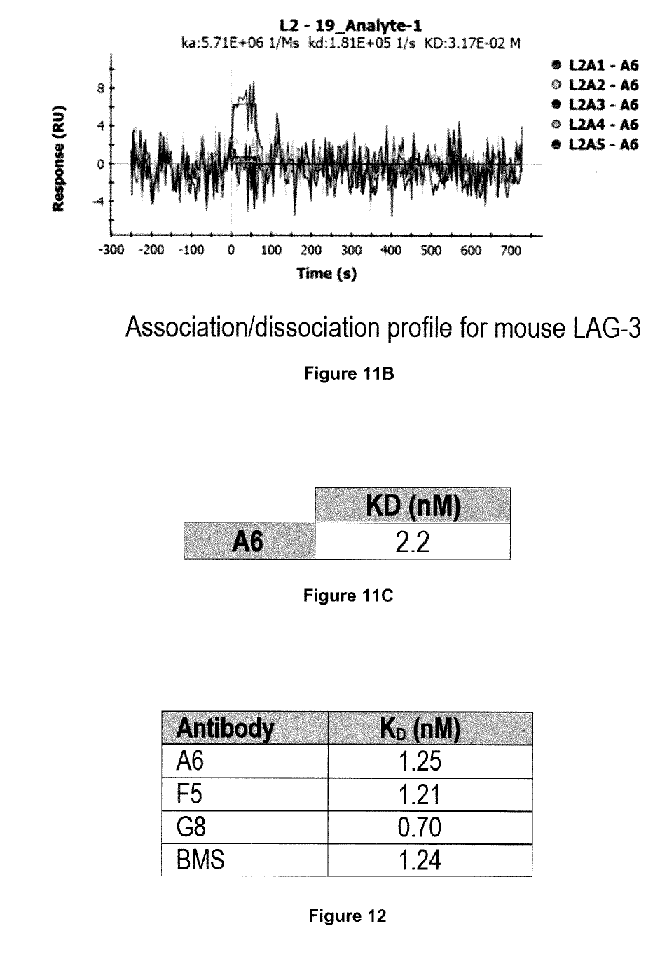

The results are shown in FIGS. 11A-C. A6 showed a slow dissociation from human LAG-3 (FIG. 11A); nevertheless, cross-binding to murine LAG-3 was not confirmed (FIG. 11B). Affinity of antibody clone A6 Fab for human LAG-3 is shown in FIG. 11C.

In a separate analysis, affinity of antibodies A6, F5, and G8 to human LAG-3 was measured using Bio-Layer Interferometry, compared to reference anti-LAG-3 antibody BMS-986016. The results are shown in FIG. 12. All of antibodies A6, F5, and G8 are shown to have high affinity for human LAG-3, and antibodies F5 and G8 in particular are shown to display a higher affinity for human LAG-3 than BMS-986016.

### Example 8: Inhibition Association of LAG-3 with MHC Class II

Anti-LAG-3 Antibodies were tested for their ability to inhibit the binding of LAG-3 to MHC class II expressed at the surface of Daudi cells.

Briefly, human LAG-3 coupled to phycoerythrin was pre-incubated for 30 minutes at room temperature with various concentrations of antibodies in FACS buffer. Daudi cells were plated in 96-well plates and fixed/permeabilised in Fix/Perm buffer in the presence of anti-CD16/CD32 antibody. Premixes were added onto the Daudi cells and incubated for 30 minutes at 4° C. The cells were then washed three times in Perm/Wash buffer, resuspended in PBS and analysed by flow cytometry.

The ability of the antibodies to block the LAG-3/MHC-II binding was calculated by determining the proportion of cells stained with phycoerythrin:

$$\frac{mean {MFI}_{negative control} - {MFI}_{tested antibody}}{mean {MFI}_{negative control}} \%$$

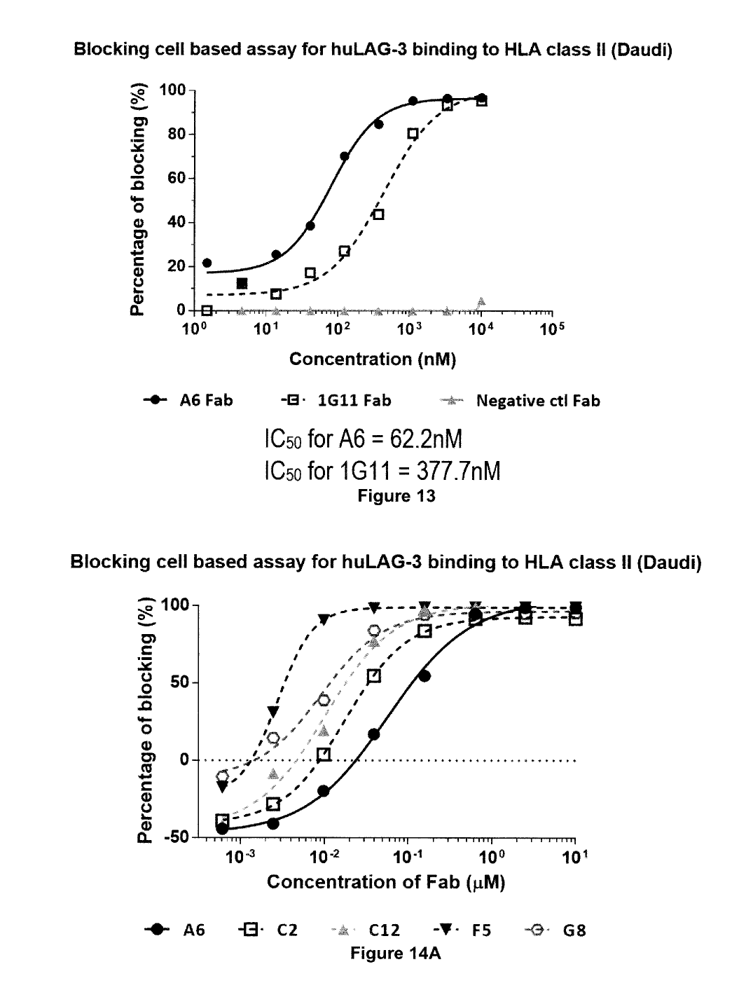

Both A6 and 1G11 antibodies showed inhibitory capacity in a dose dependent manner, and were able to completely block binding of LAG-3 to MHC-II at high concentrations (FIG. 13). Based on the data, half-maximal inhibitory concentration (IC₅₀) values for inhibiting association of LAG-3 and MHC class II were determined for antibodies A6 and 1G11. A6 was determined to have an IC₅₀ of 62.2 nM, and 1G11 was determined to have an IC₅₀ of 377.7 nM.

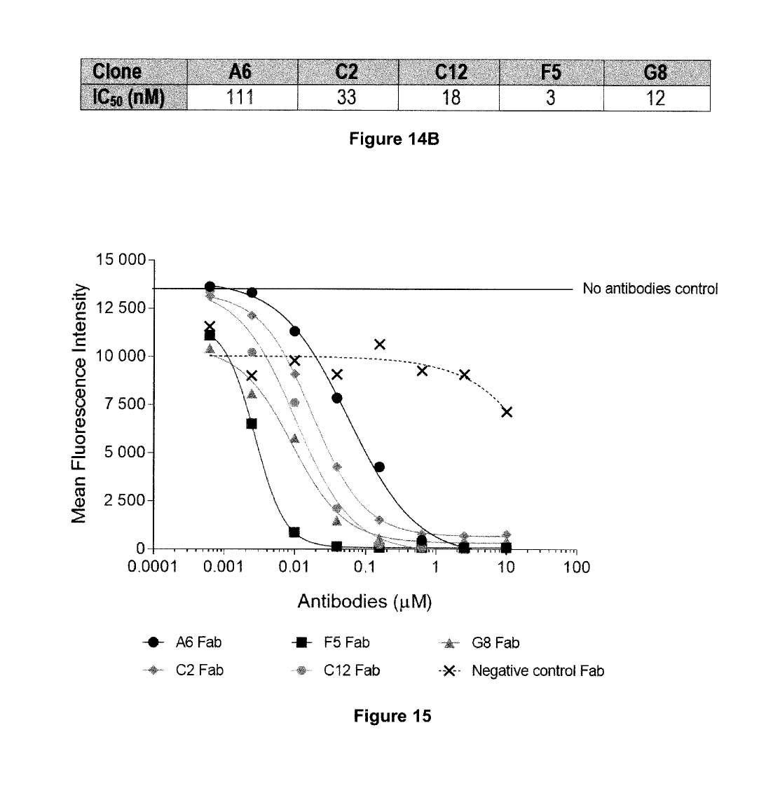

In a separate analysis, antibody clones A6, F5, and G8 were analysed for their ability to inhibit the binding of LAG-3 to MHC class II as described above. Antibody clones A6, F5, and G8 showed inhibitory capacity in a dose dependent manner, and were able to completely block binding of LAG-3 to MHC-II at high concentrations (FIG. 14A). Based on the data, IC₅₀ values for inhibiting association of LAG-3 and MHC class II were determined; the results are shown in FIG. 14B.

In a further analysis, antibody clones A6, C2, C12, F5, and G8 were analysed for their ability to inhibit the binding of LAG-3 to MHC class II. The ability of the antibodies to block the binding of LAG-3 to its ligand on Daudi cells was assessed by flow cytometry. Labelled LAG-3 was preincubated with the anti-LAG-3 Fab antibodies or with a negative Fab control prior to being added onto Daudi cells. After 30 min incubation, cells were analysed by FACS. The results are shown in FIG. 15. Antibody clones A6, C2, C12, F5, and G8 were shown to block binding of LAG-3 to MHC class 11-expressing Daudi cells in a dose-dependent manner.

### Example 9: Restoring T Cells Activity in Mixed Lymphocyte Reactions after T Cell Exhaustion

After exhaustion T cells become unresponsive to stimulation. F5 and G8 antibodies were tested for their ability to reverse exhaustion and restore the activity of T cells to secrete IL-2 and IFN-γ upon restimulation. Briefly, T cells from one donor were mixed with antigen presenting cells from an HLA-mismatched donor in a mixed lymphocyte reaction for 7 days to drive exhaustion. Exhausted cells were then restimulated with HLA-mismatched cells in the presence of anti-LAG-3 antibodies or control antibody at various concentrations, and secretion of IL-2 and IFN-γ were measured as markers of activation.

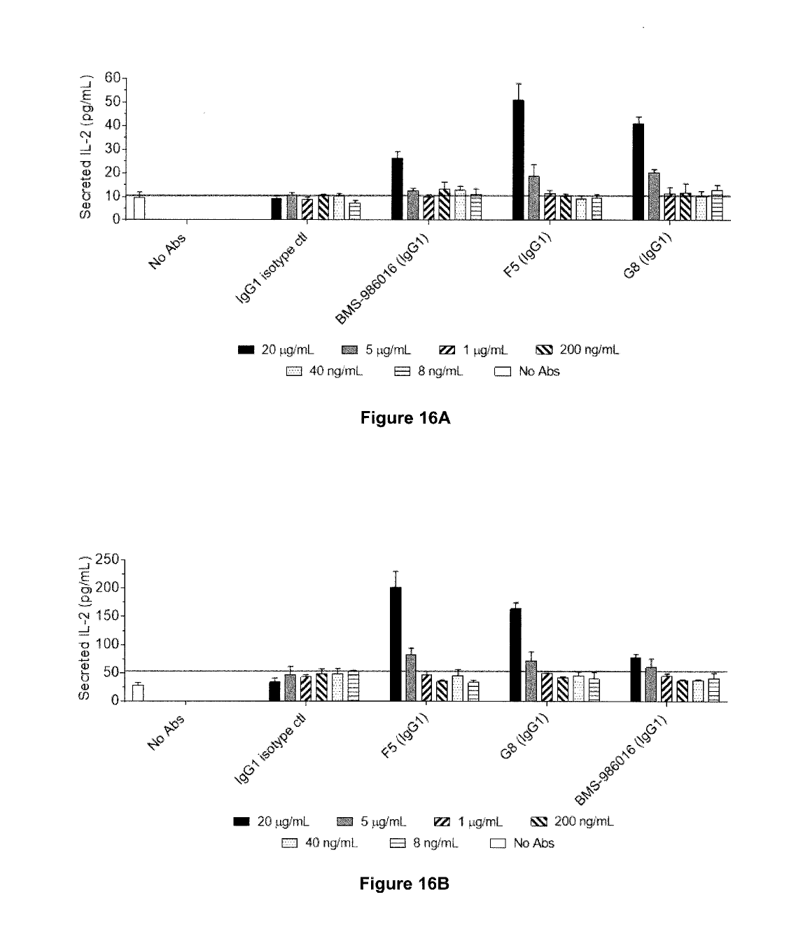

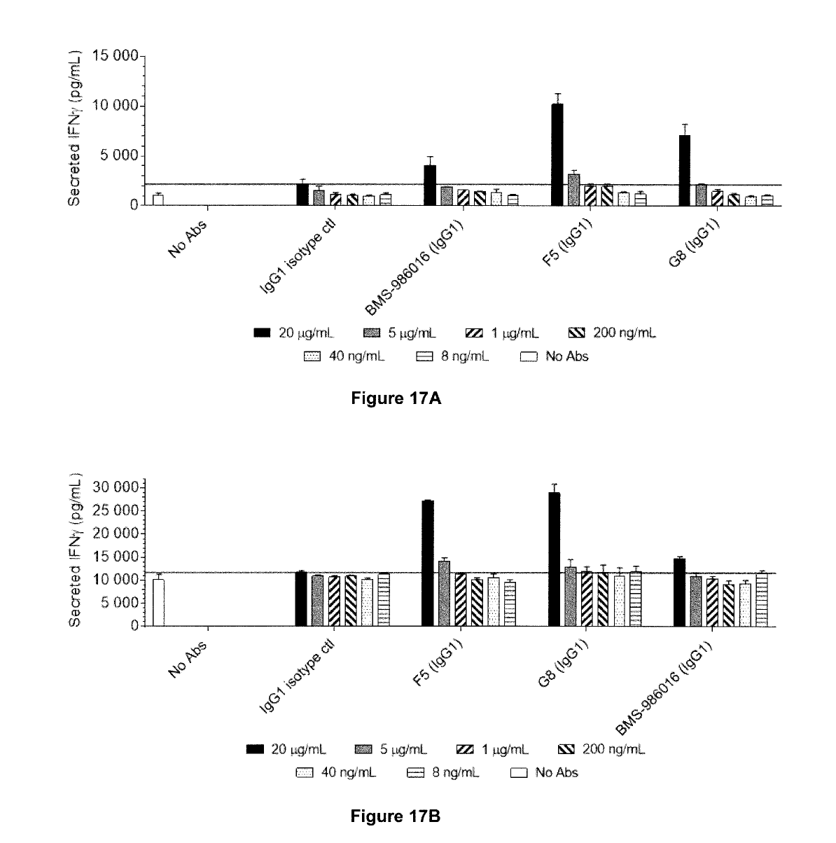

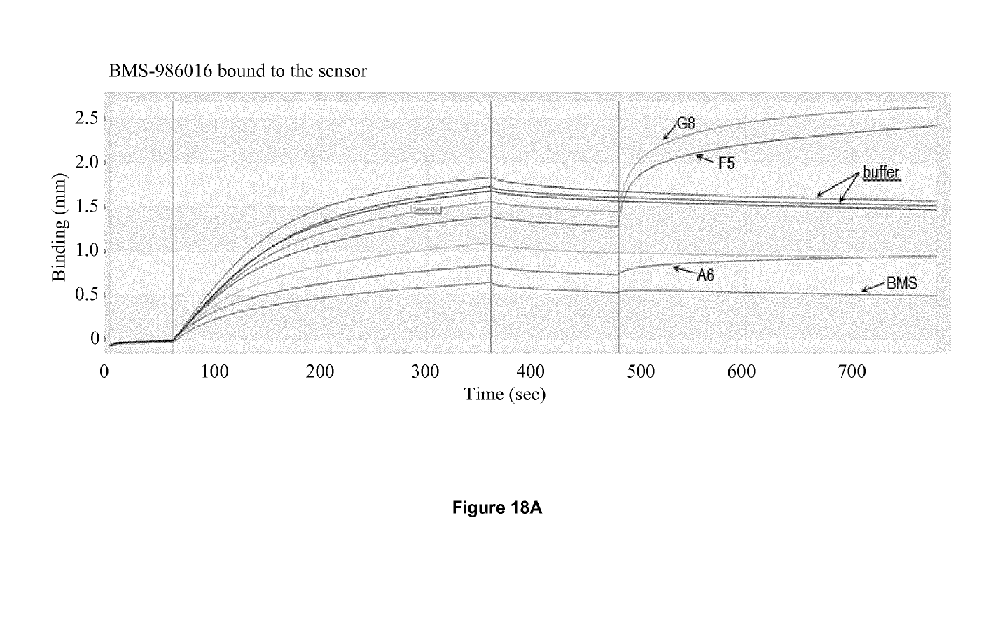

FIGS. 16A-B and 17A-B present the amount of IL-2 (FIGS. 16A-B) and IFN-γ (FIG. 17A-B) in two independent experiments (mean±SD of triplicates is shown). The black line represents the maximum mean background detected in the presence of the isotype control. F5 and G8 are able, at least at high doses, to restore T cell activity.

Antibodies F5 and G8 show better efficacy to restore T cell function than reference anti-LAG-3 antibody BMS-986016.

### Example 10: Preliminary Epitope Mapping

Bio-Layer Interferometry was used to investigate whether the different anti-LAG-3 antibody clones bind to different epitopes. In these experiments, one antibody was bound to the sensor, and LAG-3 was then flown over and allowed to bind to the bound antibody. Some buffer was run to rinse off unbound antibody. A second antibody was then applied, and binding of this second antibody to LAG-3 was analysed. The stronger the binding by the second antibody, the further away the epitope for the second antibody was determined to be from the epitope for the first antibody.

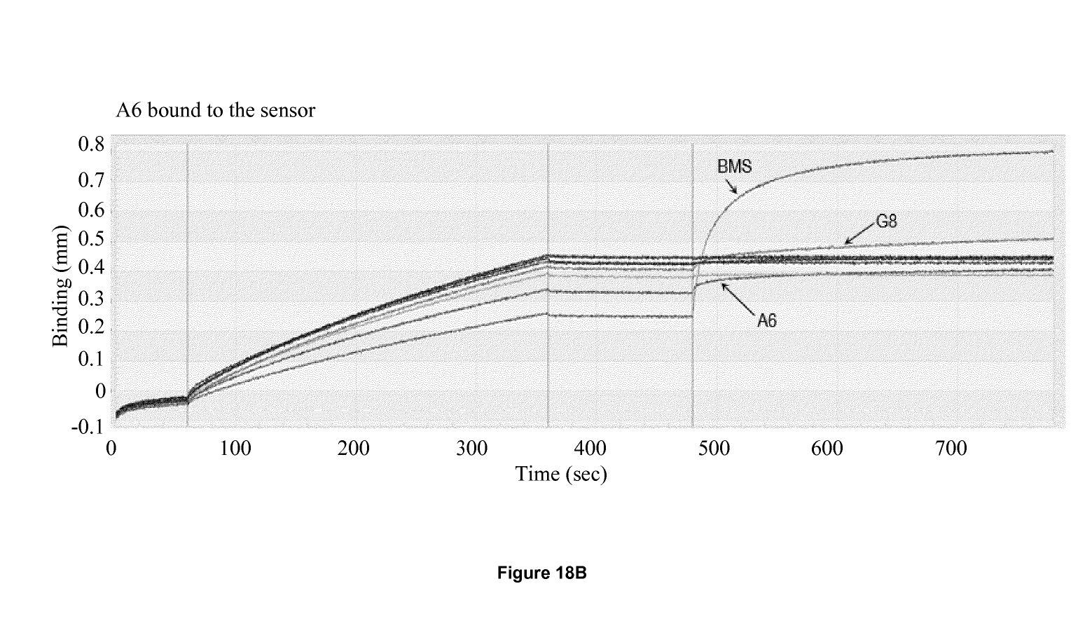

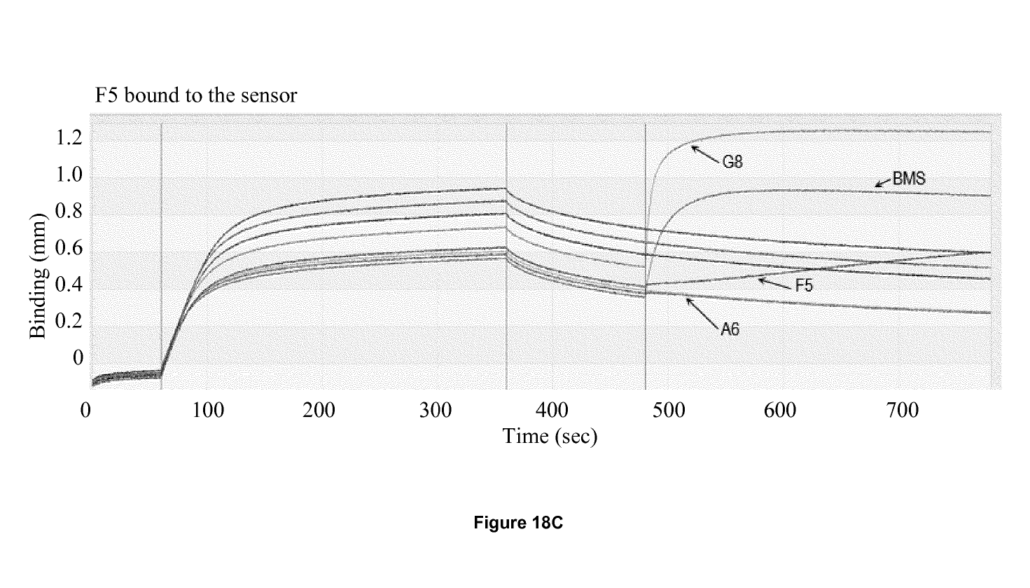

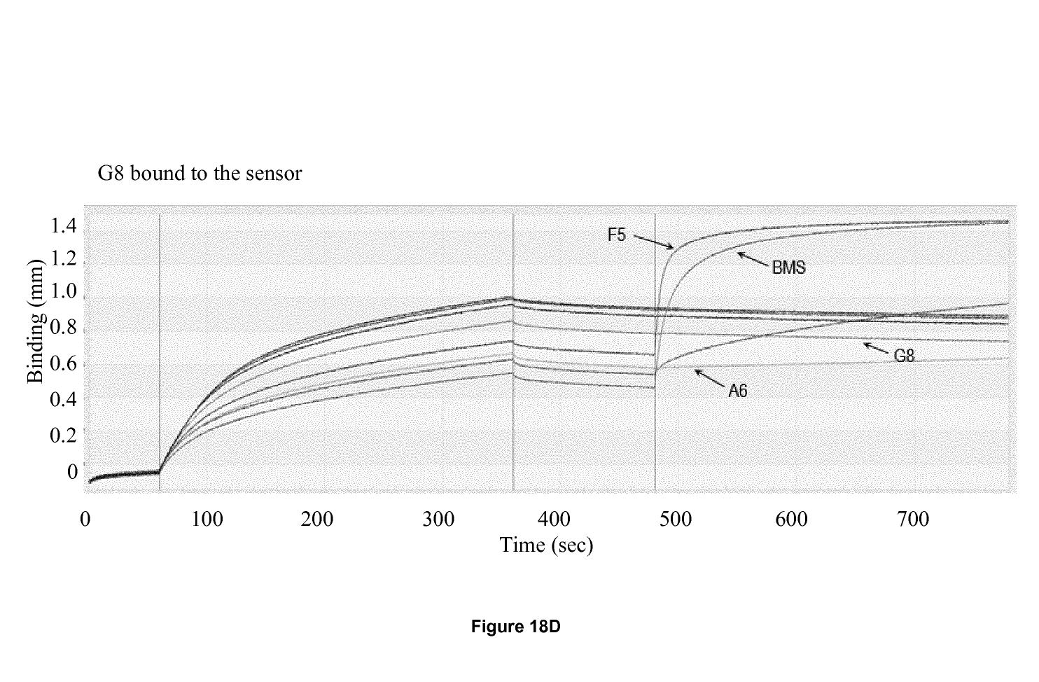

FIGS. 18A-D presents the binding profiles of the indicated antibodies to LAG-3 bound to BMS-986016 (FIG. 18A), A6 (FIG. 18B), F5 (FIG. 18C) or G8 (FIG. 18D). These profiles suggest that antibody clones A6, F5 and G8 bind to LAG-3 at a different epitope to the epitope for BMS-986016. Also, antibody clones F5 and G8 are clearly shown to have different epitopes.

### Example 11: Expansion of T Cells in the Presence of Anti-LAGS

The influence of anti-LAG-3 antibody on T cell expansion was analysed. The anti-LAG-3 antibody used in the following experiments was anti-LAG-3 antibody clone F5, in IgG1 format.

Briefly, peripheral blood mononuclear cells (PBMCs) from two different donors (ID1 and ID2) were added at 0.5×10⁶ cells/ml to wells of a 24-well cell culture plate (1 ml/well), and anti-CD3/CD28 Dynabeads were added to wells.

Recombinant human IL-2 and anti-LAG-3 antibody clone F5-IgG1 were then added to wells, to establish the following conditions:
(i) IL-2 (100 U/ml)
(ii) IL-2 (100 U/ml)+anti-LAG-3 (10 μg/ml)
(iii) IL-2 (100 U/ml)+anti-LAG-3 (1 μg/ml)
(iv) IL-2 (100 U/ml)+anti-LAG-3 (0.1 μg/ml)
(v) IL-2 (100 U/ml)+anti-LAG-3 (0.01 μg/ml)
(vi) none (beads only control)

On days 3 and 5, 0.5 ml of the culture media was removed, and 1 ml of fresh cell culture medium as added.

On day 7, cells were harvested, stained with antibodies for different cell surface markers, and then analysed by flow cytometry for different cell subsets. The results were normalised to the ‘beads only control’ group. Comparison between the different conditions was performed by ANOVA.

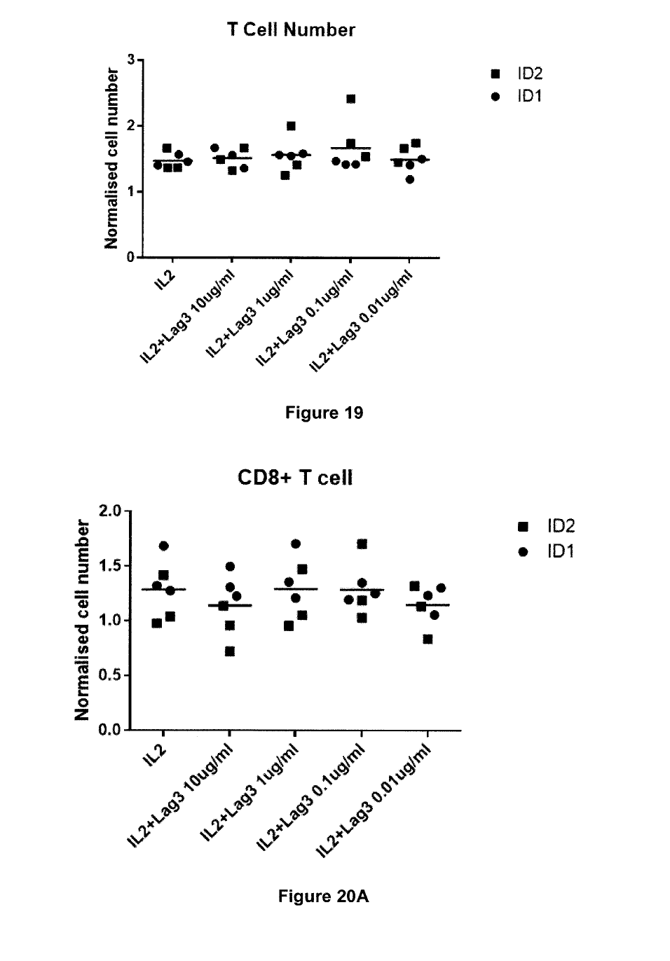

The results of the experiment are shown in FIGS. 19 to 28.

FIG. 19 shows that expansion of T cells by culture with anti-CD3/CD28 beads in the presence of IL-2 and anti-LAG-3 antibody did not change the number of T cells as compared to culture in the absence of the LAG-3 antibody (i.e. culture with anti-CD3/CD28 beads in the presence of IL-2 in the absence of anti-LAG-3 antibody).

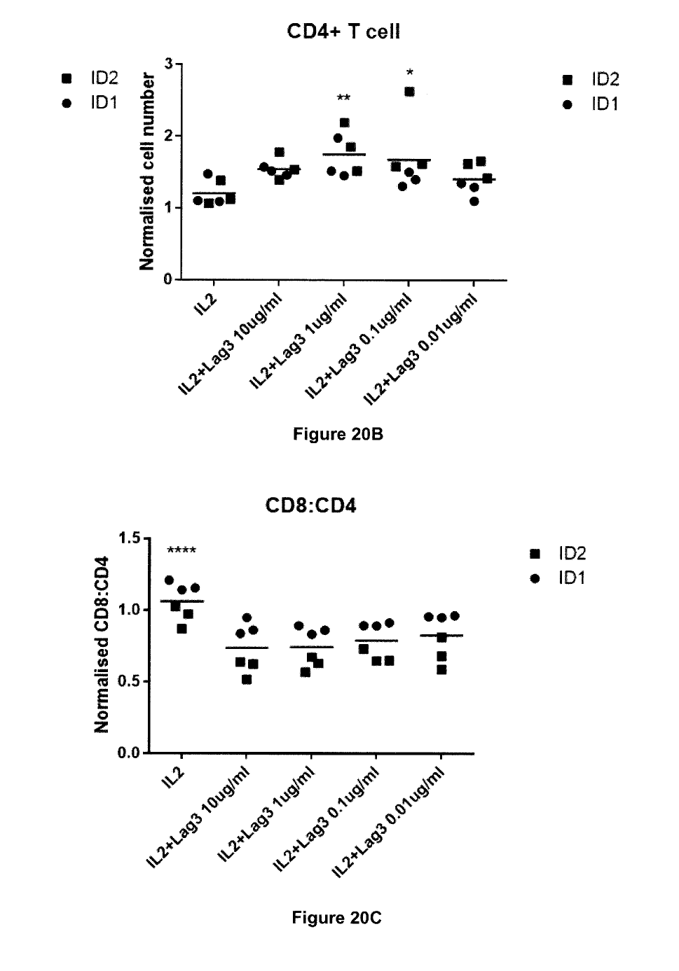

FIGS. 20A and 20B show that no significant difference was observed in the total number of CD8+ T cells expanded under the different conditions, but that a significant increase in the number of CD4+ T cells was observed for cells expanded in the presence of 1 μg/ml and 0.1 μg/ml anti-LAG-3 antibody.

FIG. 20C shows that cells expanded in the presence of anti-LAG-3 antibody had a significantly lower ratio of CD8:CD4 cells as compared to cells expanded in the absence of anti-LAG-3 antibody.

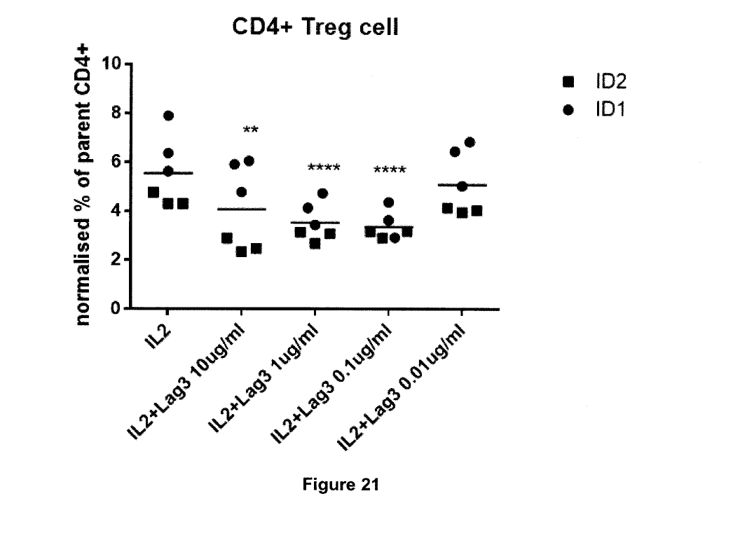

FIG. 21 shows that cells expanded in the presence of anti-LAG-3 antibody had a lower percentage of CD4+CD25+FoxP3+ Tregs within the CD4+ T cell population.

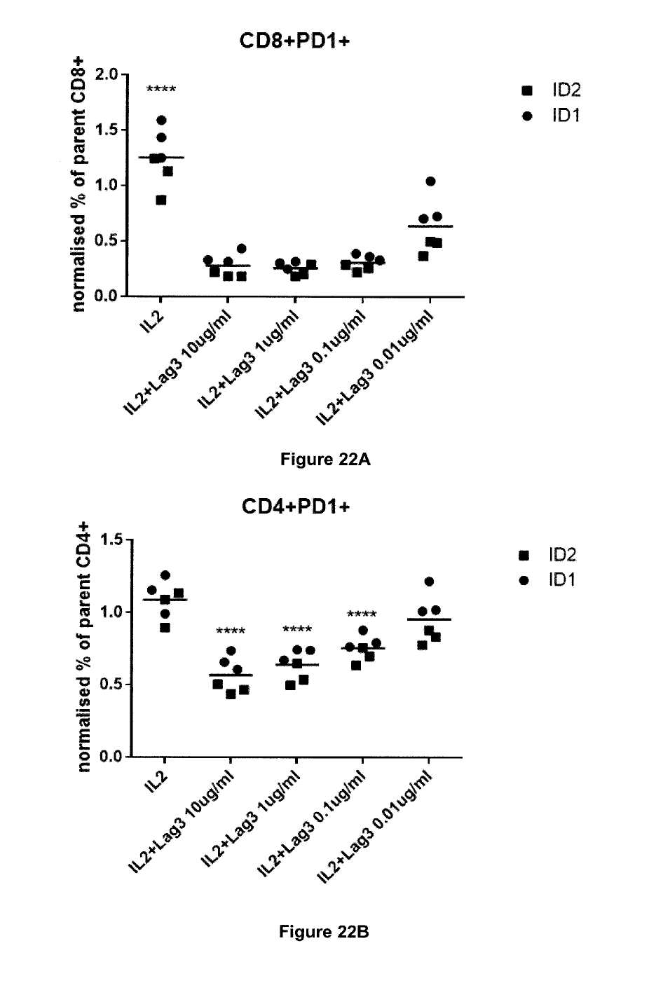

FIGS. 22A and 22B show that cells expanded in the presence of anti-LAG-3 antibody had a lower percentage of CD8+PD1+ cells within the CD8+ T cell population, and a lower percentage of CD4+PD1+ cells within the CD4+ T cell population.

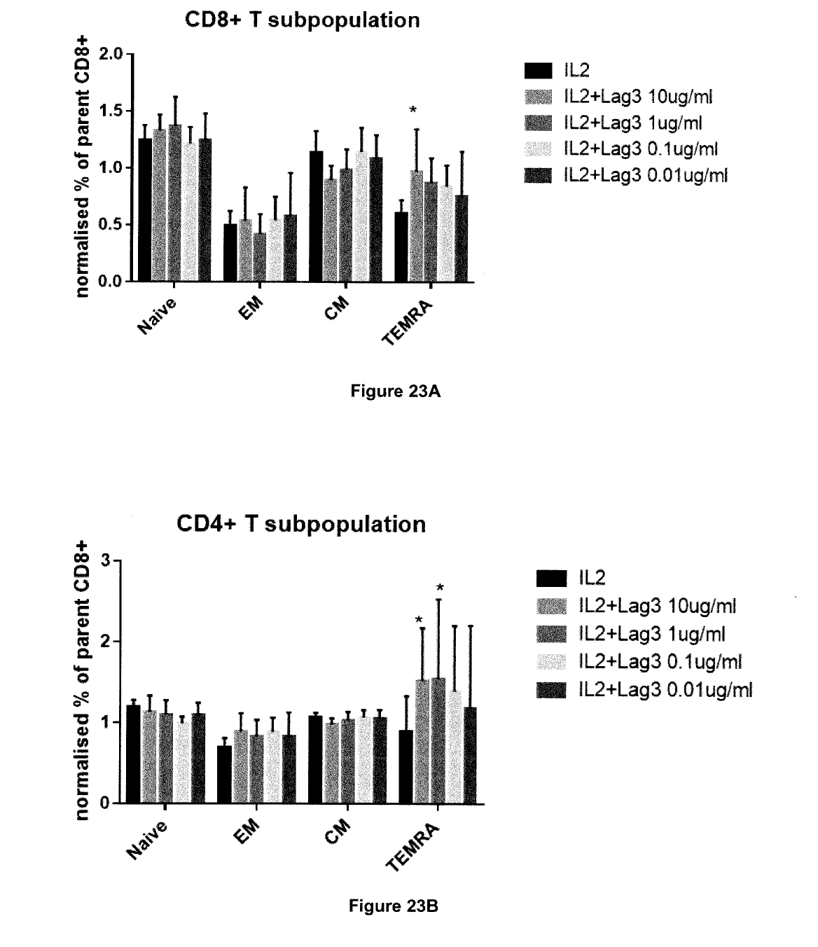

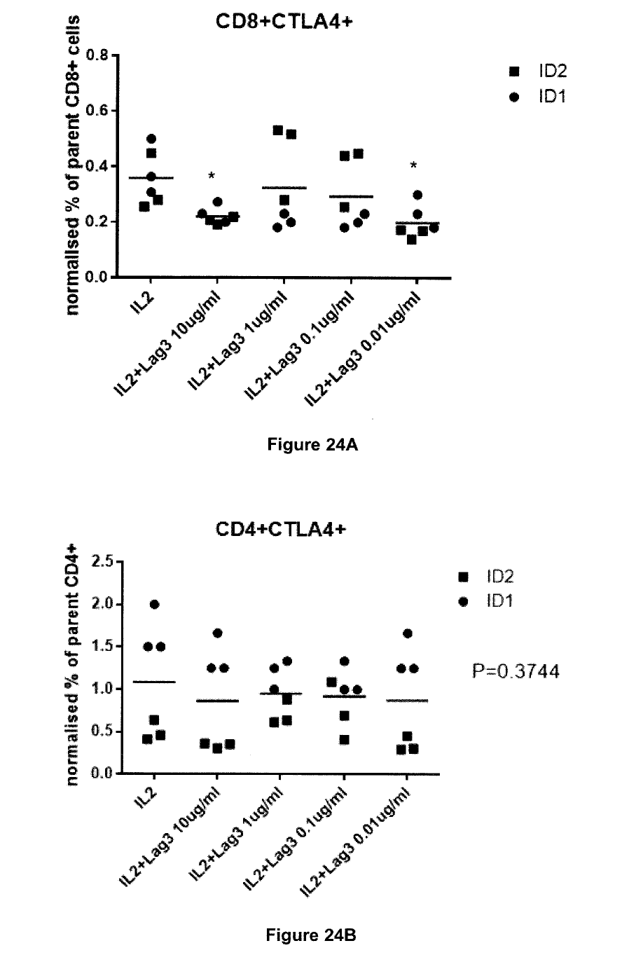

FIGS. 23A and 24B show the percentage of different T cell subpopulations within the CD8+ and CD4+ T cell populations for the cells expanded under different conditions. Cells expanded in the presence of anti-LAG-3 antibody had a higher percentage of TEMRA cells within the CD4+ and CD8+ populations.

FIGS. 24A and 24B show that cells expanded in the presence of anti-LAG-3 antibody had a slightly lower percentage of CD8+CTLA4+ cells within the CD8+ T cell population, but not in the CD4+ T cell population.

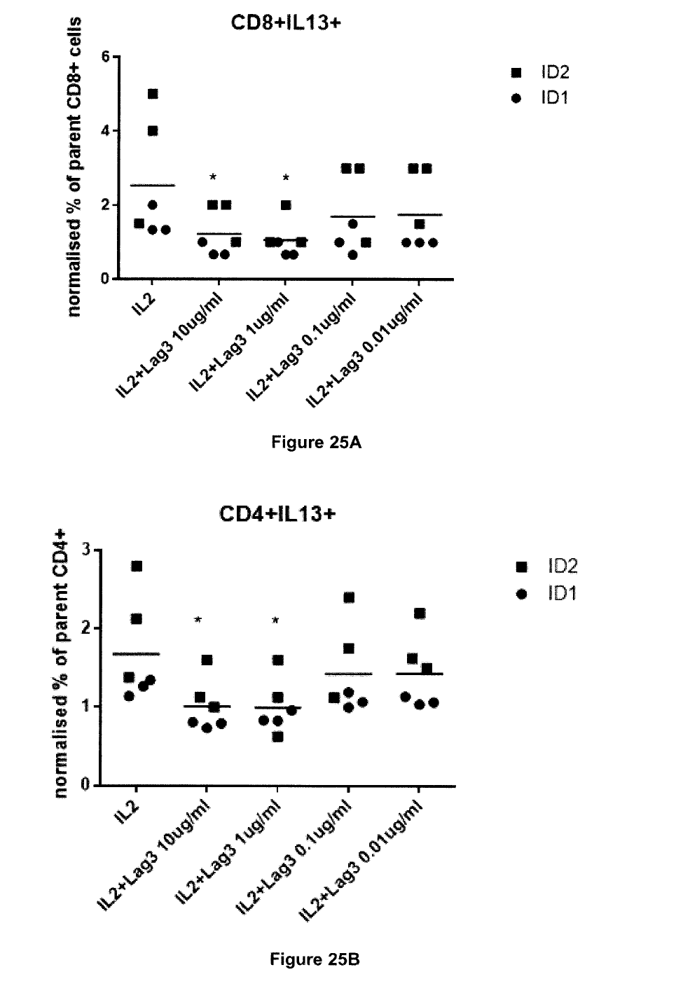

FIGS. 25A and 25B show that cells expanded in the presence of anti-LAG-3 antibody had a lower percentage of CD8+IL-13+ cells within the CD8+ T cell population, and a lower percentage of CD4+IL-13+ cells within the CD4+ T cell population.

Generally, the percentage of IL-13+ cells was low (<5%).

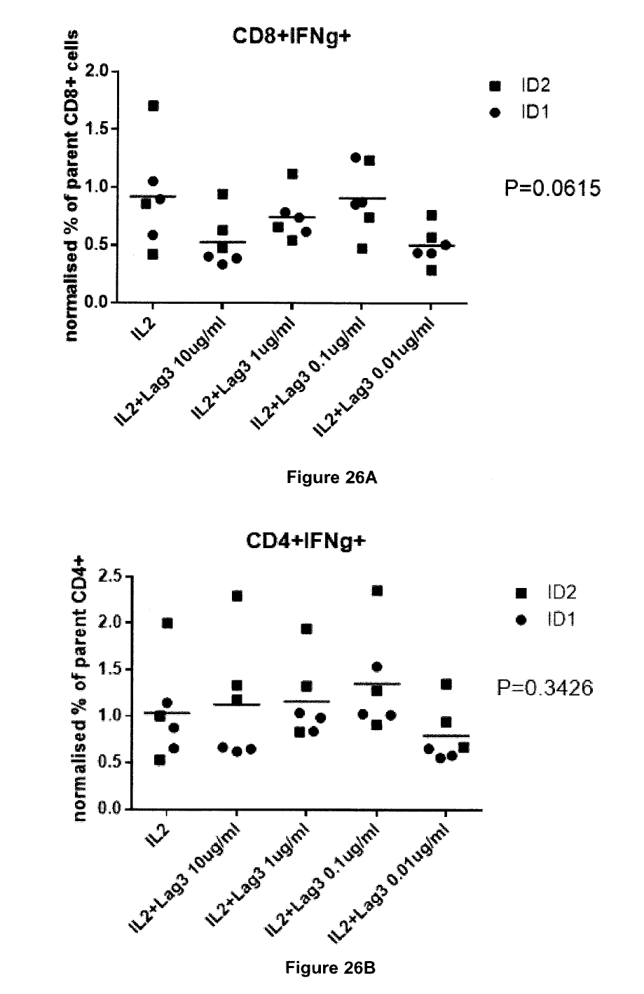

FIGS. 26A and 26B show that no difference was observed in the percentage of CD8+IFNγ+ cells within the CD8+ T cell population, nor in the percentage of CD4+IFNγ+ cells within the CD4+ T cell population.

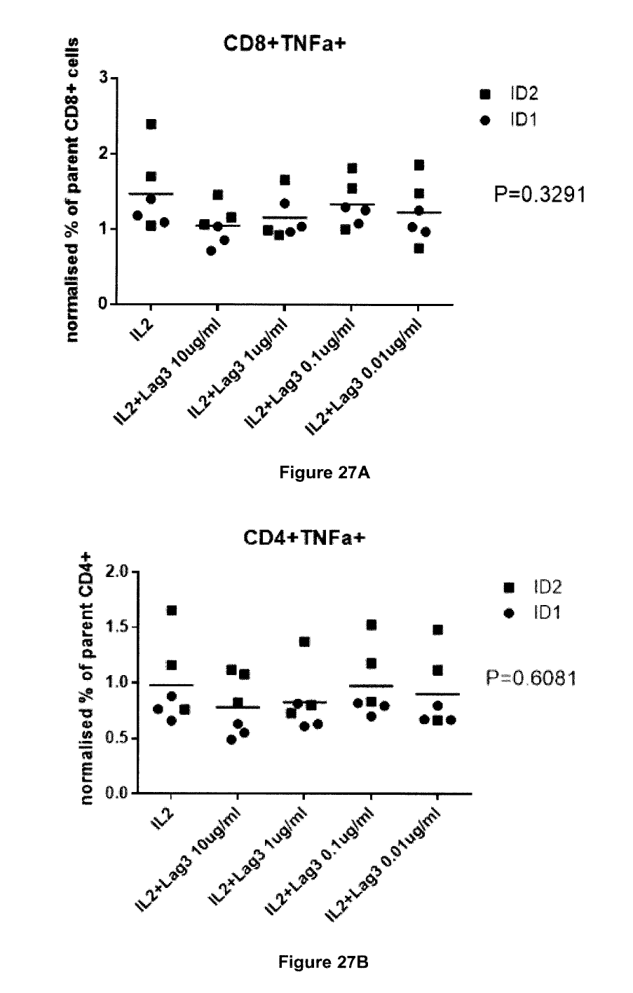

FIGS. 27A and 27B show that no difference was observed in the percentage of CD8+TNFα+ cells within the CD8+ T cell population, nor in the percentage of CD4+TNFα+ cells within the CD4+ T cell population.

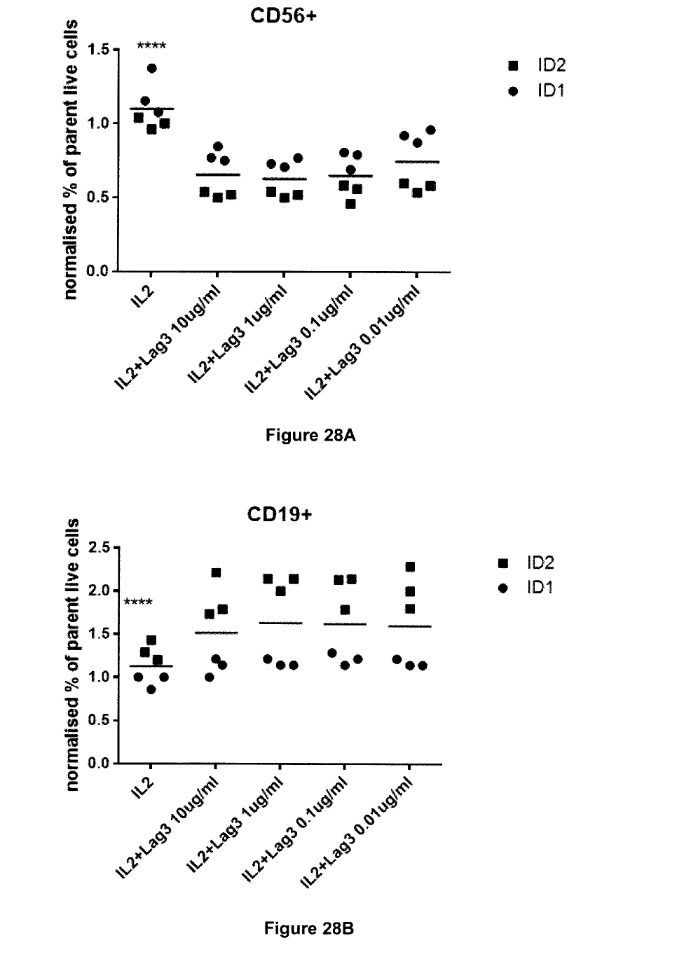

FIGS. 28A and 28B show that within the non-T cell population of the expanded cells, cells expanded in the presence of anti-LAG-3 antibody had a lower percentage of CD56+ cells (i.e. NK cells), and a higher percentage of CD19+ cells (i.e. B cells).

Generally, the percentage of CD56+ and CD19+ cells were low (<5%) for all groups; purity of the expanded T cell population was >90%.

Overall the results suggested that expansion in the presence of anti-LAG-3 antibody:
(a) does not influence the number of expanded cells
(b) does not influence the number of CD3+ cells within the expanded population;
(c) results in a lower ratio of CD8:CD4 cells within the expanded population;
(d) results in a lower proportion of Tregs within the expanded population;
(e) results in a lower proportion of PD1+ cells within the expanded population;
(f) does not significantly influence the proportion of CTLA4+ cells within the expanded population;
(g) does not significantly influence the proportion of T effector cells within the expanded population;
(h) does not significantly influence the proportion of cells expressing Th1 cytokines within the expanded population;
(i) results in a lower proportion of NK cells within the expanded population; and
(j) results in a higher proportion of B cells within the expanded population.

1. An antibody, or antigen binding fragment which is capable of binding to LAG-3, optionally isolated, having the amino acid sequences i) to vi):
i) LC-CDR1:
(SEQ ID NO: 53)
X₁X₂SQSX₃X₄X₅X₆X₇X₈X₉X₁₀X₁₁X₁₂X₁₃;
ii) LC-CDR2:
(SEQ ID NO: 54)
X₁₄X₁₅SX₁₆RAX₁₇;
iii) LC-CDR3:
(SEQ ID NO: 55)
X₁₈QX₁₉X₂₀X₂₁X₂₂X₂₃X₂₄X₂₅X₂₆X₂₇;
iv) HC-CDR1:
(SEQ ID NO: 56)
X₂₈X₂₉X₃₀X₃₁X₃₂;
v) HC-CDR2:
(SEQ ID NO: 57)
X₃₃X₃₄X₃₅X₃₆X₃₇X₃₈X₃₉X₄₀X₄₁X₄₂YAX₄₃X₄₄X₄₅X₄₆G;
vi) HC-CDR3:
(SEQ ID NO: 41)
one of TWFGELYY,
(SEQ ID NO: 30)
PFGDFDY,
(SEQ ID NO: 33)
LPGWGAYAFDI,
(SEQ ID NO: 35)
DPDAANWGFLLYYGMDV
or
(SEQ ID NO: 38)
ALADFWSGYYYYYYMDV;
or a variant thereof in which one or two or three amino acids in one or more of the sequences (i) to (vi) are replaced with another amino acid, where X₁=R or T; X₂=S, A or T; X₃=L or V; X₄=L or S; X₅=H or S; X₆=S, G or T; X₇=N, F, Y, D or S; X₈=G or L; X₉=Y, A or D; X₁₀=absent or N; X₁₁=absent or Y; X₁₂=absent, L or F; X₁₃=absent or D; X₁₄=L, G or D; X₁₅=G or A; X₁₆=N or S; X₁₇=S, T or A; X₁₈=M or Q; X₁₉=A, Y or G; X₂₀=L, G or T; X₂₁=Q, P, S or H; X₂₂=T, S or W; X₂₃=P, I, R or L; X₂₄=Y, T, P or L; X₂₅=absent, T, I or G; X₂₆=absent, T or L; X₂₇=absent or T; X₂₈=S or E; X₂₉=Y or L; X₃₀=Y, G, A or S; X₃₁=M or I; X₃₂=H or S; X₃₃=I, G or V; X₃₄=I or F; X₃₅=N, S, I or D; X₃₆=P or Y; X₃₇=S, D, I or E; X₃₈=G, F or D; X₃₉=G or S; X₄₀=S, N, T or E; X₄₁=T, K or A; X₄₂=S, Y, N or I; X₄₃=Q or D; X₄₄=K or S; X₄₅=F or V; and X₄₆ is Q or K.

2. The antibody, or antigen binding fragment, of claim 1, wherein LC-CDR1 is one of RASQSVSSGYLA (SEQ ID NO:23), RSSQSLLHSNGYNYLD (SEQ ID NO:12), RASQSVSSSFLA (SEQ ID NO:15), RASQSVSSSYLA (SEQ ID NO:18), RSSQSLLHSDGYNYFD (SEQ ID NO:20) or TTSQSVSSTSLD (SEQ ID NO:26).

3. The antibody, or antigen binding fragment, of claim 1 or claim 2, wherein LC-CDR2 is one of DASSRAT (SEQ ID NO:24), LGSNRAS (SEQ ID NO:13), GASSRAT (SEQ ID NO:16) or LGSNRAA (SEQ ID NO:21).

4. The antibody, or antigen binding fragment, of any one of claims 1 to 3, wherein LC-CDR3 is one of QQYGSSRPGLT (SEQ ID NO:25), MQALQTPYT (SEQ ID NO:14), QQYGPSIT (SEQ ID NO:17), QQYGSSPPIT (SEQ ID NO:19), MQGTHWPPT (SEQ ID NO:22) or QQYGSSLLT (SEQ ID NO:27).

5. The antibody, or antigen binding fragment, of any one of claims 1 to 4, wherein HC-CDR1 is one of ELSMH (SEQ ID NO:39), SYYMH (SEQ ID NO:28), SYGMH (SEQ ID NO:31), SYAMH (SEQ ID NO:34) or SYAIS (SEQ ID NO:36).

6. The antibody, or antigen binding fragment, of any one of claims 1 to 5, wherein HC-CDR2 is one of GFDPEDGETIYAQKFQG (SEQ ID NO:40), IINPSGGSTSYAQKFQG (SEQ ID NO:29) VISYDGSNKYYADSVKG (SEQ ID NO:32) or GIIPIFGTANYAQKFQG (SEQ ID NO:37).

7. The antibody, or antigen binding fragment, of any one of claims 1 to 6, having at least one light chain variable region incorporating the following CDRs:
(SEQ ID NO: 53)
LC-CDR1: X₁X₂SQSX₃X₄X₅X₆X₇X₈X₉X₁₀X₁₁X₁₂X₁₃
(SEQ ID NO: 54)
LC-CDR2: X₁₄X₁₅SX₁₆RAX₁₇
(SEQ ID NO: 55)
LC-CDR3: X₁₈QX₁₉X₂₀X₂₁X₂₂X₂₃X₂₄X₂₅X₂₆X₂₇;
where X₁=R or T; X₂=S, A or T; X₃=L or V; X₄=L or S; X₅=H or S; X₆=S, G or T; X₇=N, F, Y, D or S; X₈=G or L; X₉=Y, A or D; X₁₀=absent or N; X₁₁=absent or Y; X₁₂=absent, L or F; X₁₃=absent or D; X₁₄=L, G or D; X₁₅=G or A; X₁₆=N or S; X₁₇=S, T or A; X₁₈=M or Q; X₁₉=A, Y or G; X₂₀=L, G or T; X₂₁=Q, P, S or H; X₂₂=T, S or W; X₂₃=P, I, R or L; X₂₄=Y, T, P or L; X₂₅=absent, T, I or G; X₂₆=absent, T or L; and X₂₇=absent or T.

8. The antibody, or antigen binding fragment, of any one of claims 1 to 7, having at least one light chain variable region incorporating the following CDRs:
(SEQ ID NO: 23)
LC-CDR1: RASQSVSSGYLA
(SEQ ID NO: 24)
LC-CDR2: DASSRAT
(SEQ ID NO: 25)
LC-CDR3: QQYGSSRPGLT.

9. The antibody, or antigen binding fragment, of any one of claims 1 to 7, having at least one light chain variable region incorporating the following CDRs:
(SEQ ID NO: 12)
LC-CDR1: RSSQSLLHSNGYNYLD
(SEQ ID NO: 13)
LC-CDR2: LGSNRAS
(SEQ ID NO: 14)
LC-CDR3: MQALQTPYT.

10. The antibody, or antigen binding fragment, of any one of claims 1 to 7, having at least one light chain variable region incorporating the following CDRs:
(SEQ ID NO: 15)
LC-CDR1: RASQSVSSSFLA
(SEQ ID NO: 16)
LC-CDR2: GASSRAT
(SEQ ID NO: 17)
LC-CDR3: QQYGPSIT.

11. The antibody, or antigen binding fragment, of any one of claims 1 to 7, having at least one light chain variable region incorporating the following CDRs:
(SEQ ID NO: 18)
LC-CDR1: RASQSVSSSYLA
(SEQ ID NO: 16)
LC-CDR2: GASSRAT
(SEQ ID NO: 19)
LC-CDR3: QQYGSSPPIT.

12. The antibody, or antigen binding fragment, of any one of claims 1 to 7, having at least one light chain variable region incorporating the following CDRs:
(SEQ ID NO: 20)
LC-CDR1: RSSQSLLHSDGYNYFD
(SEQ ID NO: 21)
LC-CDR2: LGSNRAA
(SEQ ID NO: 22)
LC-CDR3: MQGTHWPPT.

13. The antibody, or antigen binding fragment, of any one of claims 1 to 7, having at least one light chain variable region incorporating the following CDRs:
(SEQ ID NO: 26)
LC-CDR1: TTSQSVSSTSLD
(SEQ ID NO: 16)
LC-CDR2: GASSRAT
(SEQ ID NO: 27)
LC-CDR3: QQYGSSLLT.

14. The antibody, or antigen binding fragment, of any one of claims 1 to 13, having at least one heavy chain variable region incorporating the following CDRs:
HC-CDR1:
(SEQ ID NO: 56)
X₂₈X₂₉X₃₀X₃₁X₃₂;
HC-CDR2:
(SEQ ID NO: 57)
X₃₃X₃₄X₃₅X₃₆X₃₇X₃₈X₃₉X₄₀X₄₁X₄₂YAX₄₃X₄₄X₄₅X₄₆G;
HC-CDR3:
(SEQ ID NO: 41)
one of TWFGELYY,
(SEQ ID NO: 30)
PFGDFDY,
(SEQ ID NO: 33)
LPGWGAYAFDI,
(SEQ ID NO: 35)
DPDAANWGFLLYYGMDV
or
(SEQ ID NO: 38)
ALADFWSGYYYYYYMDV;
where X₂₈=S or E; X₂₉=Y or L; X₃₀=Y, G, A or S; X₃₁=M or I; X₃₂=H or S; X₃₃=I, G or V; X₃₄=I or F; X₃₅=N, S, I or D; X₃₆=P or Y; X₃₇=S, D, I or E; X₃₈=G, F or D; X₃₉=G or S; X₄₀=S, N, T or E; X₄₁=T, K or A; X₄₂=S, Y, N or I; X₄₃=Q or D; X₄₄=K or S; X₄₅=F or V; and X₄₆ is Q or K.

15. The antibody, or antigen binding fragment, of any one of claims 1 to 14, having at least one heavy chain variable region incorporating the following CDRs:
(SEQ ID NO: 39)
HC-CDR1: ELSMH
(SEQ ID NO: 40)
HC-CDR2: GFDPEDGETIYAQKFQG
(SEQ ID NO: 41)
HC-CDR3: TWFGELYY.

16. The antibody, or antigen binding fragment, of any one of claims 1 to 14, having at least one heavy chain variable region incorporating the following CDRs:
(SEQ ID NO: 28)
HC-CDR1: SYYMH
(SEQ ID NO: 29)
HC-CDR2: IINPSGGSTSYAQKFQG
(SEQ ID NO: 30)
HC-CDR3: PFGDFDY.

17. The antibody, or antigen binding fragment, of any one of claims 1 to 14, having at least one heavy chain variable region incorporating the following CDRs:
(SEQ ID NO: 31)
HC-CDR1: SYGMH
(SEQ ID NO: 32)
HC-CDR2: VISYDGSNKYYADSVKG
(SEQ ID NO: 33)
HC-CDR3: LPGWGAYAFDI.

18. The antibody, or antigen binding fragment, of any one of claims 1 to 14, having at least one heavy chain variable region incorporating the following CDRs:
(SEQ ID NO: 34)
HC-CDR1: SYAMH
(SEQ ID NO: 32)
HC-CDR2: VISYDGSNKYYADSVKG
(SEQ ID NO: 35)
HC-CDR3: DPDAANWGFLLYYGMDV.

19. The antibody, or antigen binding fragment, of any one of claims 1 to 14, having at least one heavy chain variable region incorporating the following CDRs:
(SEQ ID NO: 36)
HC-CDR1: SYAIS
(SEQ ID NO: 37)
HC-CDR2: GIIPIFGTANYAQKFQG
(SEQ ID NO: 38)
HC-CDR3: ALADFWSGYYYYYYMDV.

20. The antibody, or antigen binding fragment, according to any one of claims 1 to 19, that specifically binds to human, rhesus macaque or murine LAG-3.

21. The antibody, or antigen binding fragment, according to any one of claims 1 to 20, that inhibits interaction between LAG-3 and MHC class II, optionally human LAG-3 and human MHC class II.

22. The antibody, or antigen binding fragment, of any one of claims 1 to 21, wherein the antibody is effective to restore T-cell function in T-cells exhibiting T-cell exhaustion or T-cell anergy.

23. An isolated light chain variable region polypeptide comprising the following CDRs:
(SEQ ID NO: 53)
LC-CDR1: X₁X₂SQSX₃X₄X₅X₆X₇X₈X₉X₁₀X₁₁X₁₂X₁₃
(SEQ ID NO: 54)
LC-CDR2: X₁₄X₁₅SX₁₆RAX₁₇
(SEQ ID NO: 55)
LC-CDR3: X₁₈QX₁₉X₂₀X₂₁X₂₂X₂₃X₂₄X₂₅X₂₆X₂₇;
where X₁=R or T; X₂=S, A or T; X₃=L or V; X₄=L or S; X₆=H or S; X₆=S, G or T; X₇=N, F, Y, D or S; X₈=G or L; X₉=Y, A or D; X₁₀=absent or N; X₁₁=absent or Y; X₁₂=absent, L or F; X₁₃=absent or D; X₁₄=L, G or D; X₁₅=G or A; X₁₆=N or S; X₁₇=S, T or A; X₁₈=M or Q; X₁₉=A, Y or G; X₂₀=L, G or T; X₂₁=Q, P, S or H; X₂₂=T, S or W; X₂₃=P, I, R or L; X₂₄=Y, T, P or L; X₂₅=absent, T, I or G; X₂₆=absent, T or L; and X₂₇=absent or T.

24. The isolated light chain variable region polypeptide of claim 23, wherein LC-CDR1 is one of RASQSVSSGYLA (SEQ ID NO:23), RSSQSLLHSNGYNYLD (SEQ ID NO:12), RASQSVSSSFLA (SEQ ID NO:15), RASQSVSSSYLA (SEQ ID NO:18), RSSQSLLHSDGYNYFD (SEQ ID NO:20) or TTSQSVSSTSLD (SEQ ID NO:26).

25. The isolated light chain variable region polypeptide of claim 23 or claim 24, wherein LC-CDR2 is one of DASSRAT (SEQ ID NO:24), LGSNRAS (SEQ ID NO:13), GASSRAT (SEQ ID NO:16) or LGSNRAA (SEQ ID NO:21).

26. The isolated light chain variable region polypeptide of any one of claim 23 to claim 25, wherein LC-CDR3 is one of QQYGSSRPGLT (SEQ ID NO:25), MQALQTPYT (SEQ ID NO:14), QQYGPSIT (SEQ ID NO:17), QQYGSSPPIT (SEQ ID NO:19), MQGTHWPPT (SEQ ID NO:22) or QQYGSSLLT (SEQ ID NO:27).

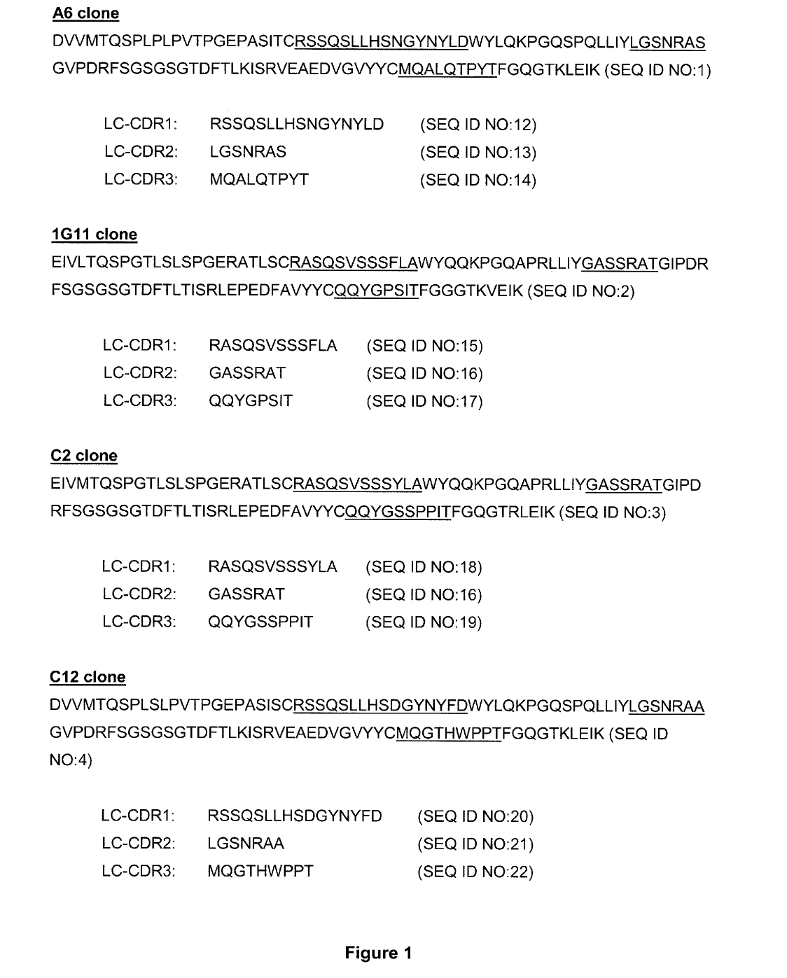

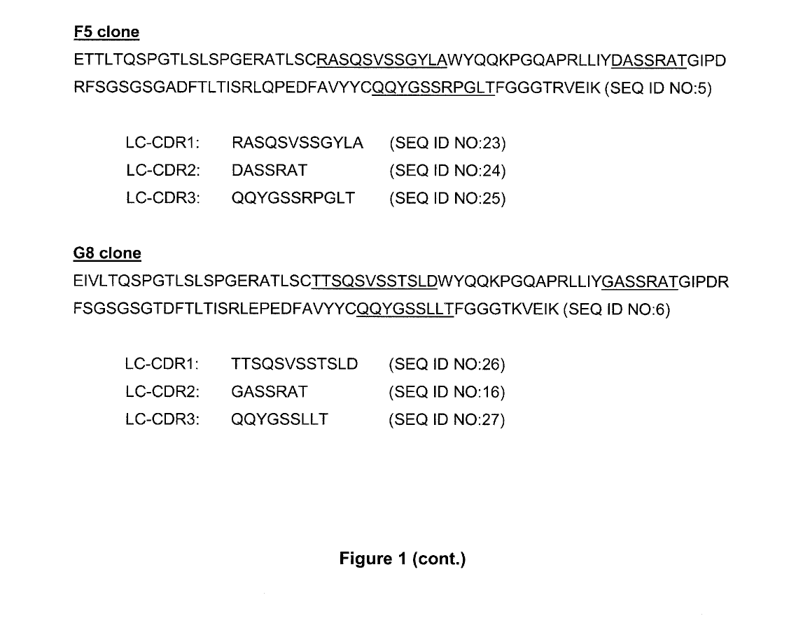

27. An isolated light chain variable region polypeptide comprising an amino acid sequence having at least 85% sequence identity to the light chain sequence: SEQ ID NO:1, 2, 3, 4, 5 or 6 (FIG. 1).

28. An isolated heavy chain variable region polypeptide comprising the following CDRs:
HC-CDR1:
(SEQ ID NO: 56)
X₂₈X₂₉X₃₀X₃₁X₃₂;
HC-CDR2:
(SEQ ID NO: 57)
X₃₃X₃₄X₃₅X₃₆X₃₇X₃₈X₃₉X₄₀X₄₁X₄₂YAX₄₃X₄₄X₄₅X₄₆G;
HC-CDR3:
(SEQ ID NO: 41)
one of TWFGELYY,
(SEQ ID NO: 30)
PFGDFDY,
(SEQ ID NO: 33)
LPGWGAYAFDI,
(SEQ ID NO: 35)
DPDAANWGFLLYYGMDV,
(SEQ ID NO: 38)
ALADFWSGYYYYYYMDV;
where X₂₈=S or E; X₂₉=Y or L; X₃₉=Y, G, A or S; X₃₁=M or I; X₃₂=H or S; X₃₃=I, G or V; X₃₄=I or F; X₃₅=N, S, I or D; X₃₆=P or Y; X₃₇=S, D, I or E; X₃₈=G, F or D; X₃₉=G or S; X₄₀=S, N, T or E; X₄₁=T, K or A; X₄₂=S, Y, N or I; X₄₃=Q or D; X₄₄=K or S; X₄₅=F or V; and X₄₆ is Q or K.

29. The isolated heavy chain variable region polypeptide of claim 28, wherein HC-CDR1 is one of ELSMH (SEQ ID NO:39), SYYMH (SEQ ID NO:28), SYGMH (SEQ ID NO:31), SYAMH (SEQ ID NO:34) or SYAIS (SEQ ID NO:36).

30. The isolated heavy chain variable region polypeptide of claim 28 or claim 29, wherein HC-CDR2 is one of GFDPEDGETIYAQKFQG (SEQ ID NO:40), IINPSGGSTSYAQKFQG (SEQ ID NO:29) VISYDGSNKYYADSVKG (SEQ ID NO:32) or GIIPIFGTANYAQKFQG (SEQ ID NO:37).

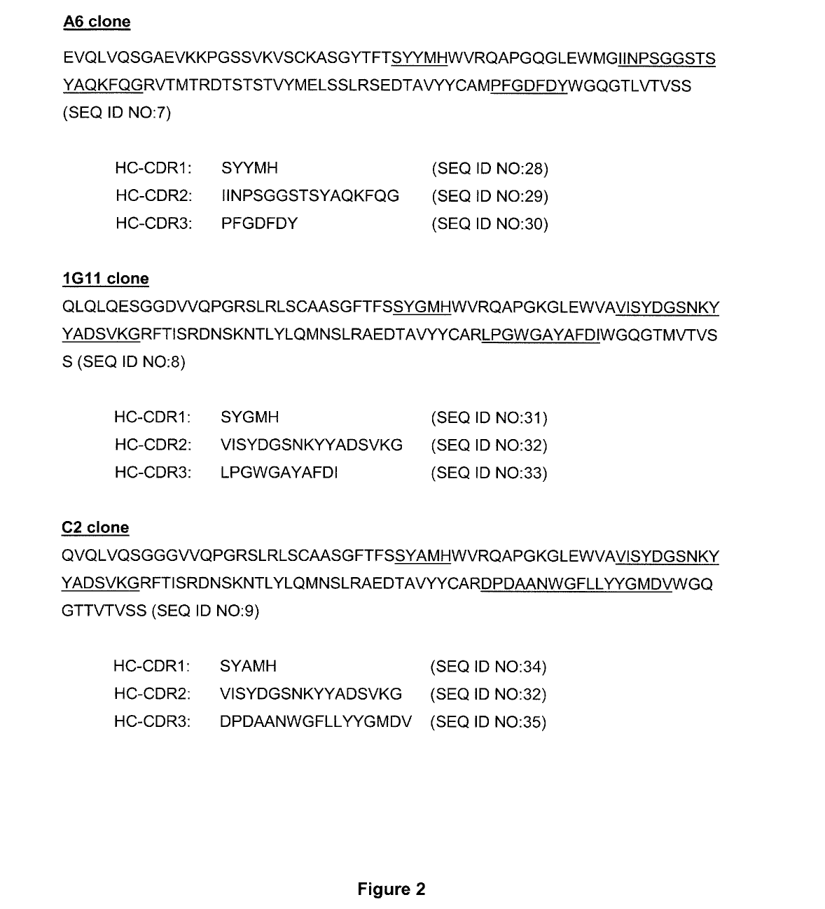

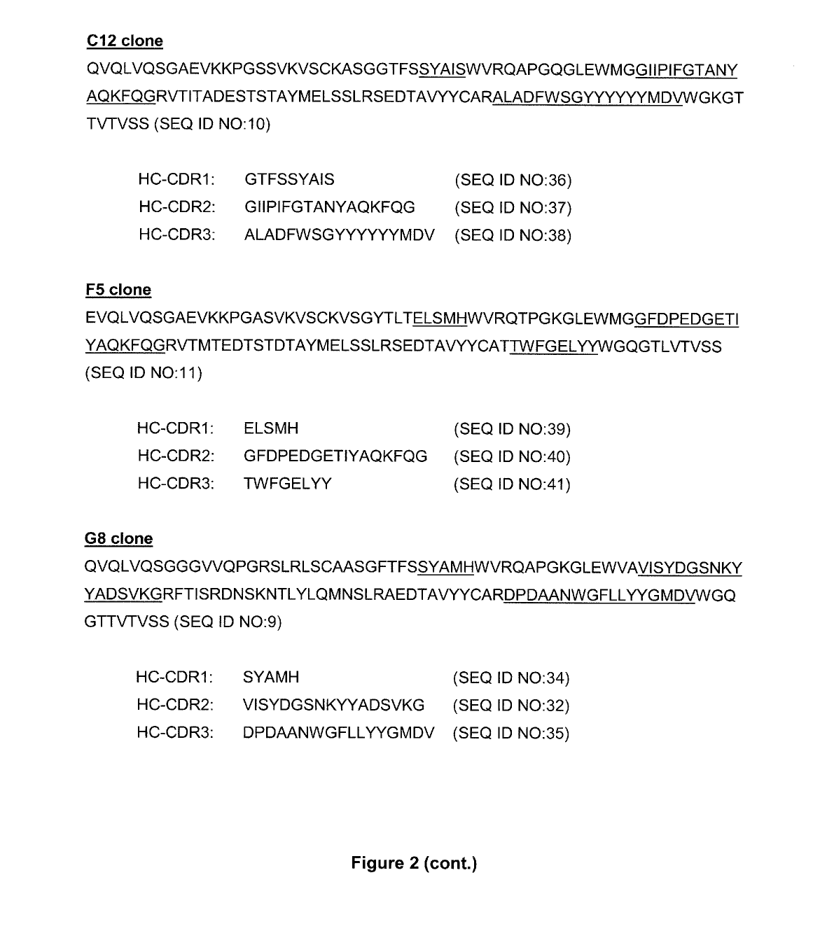

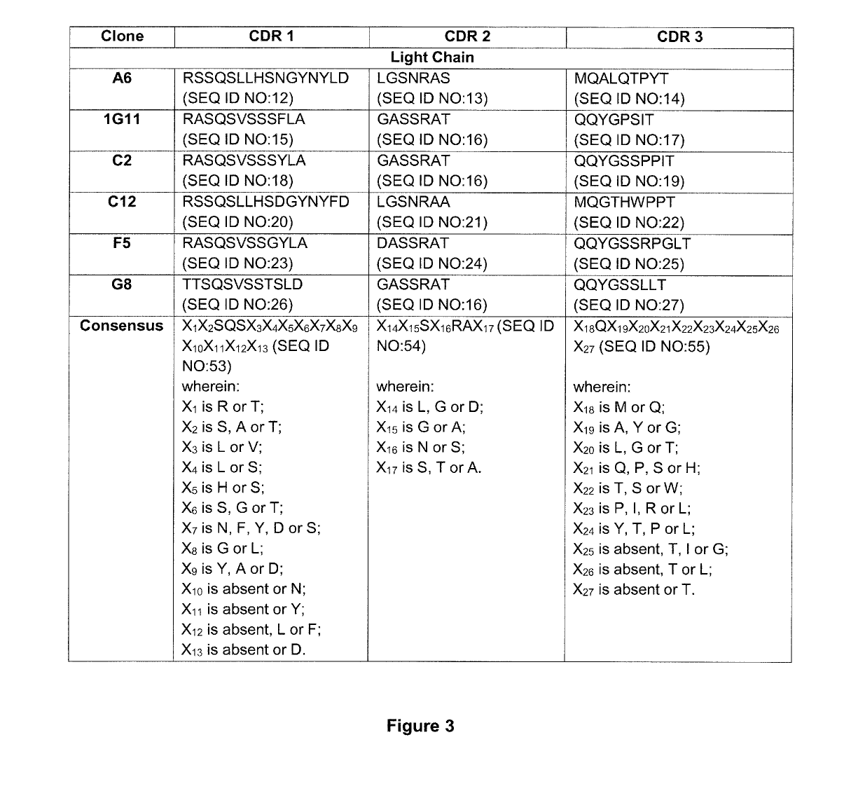

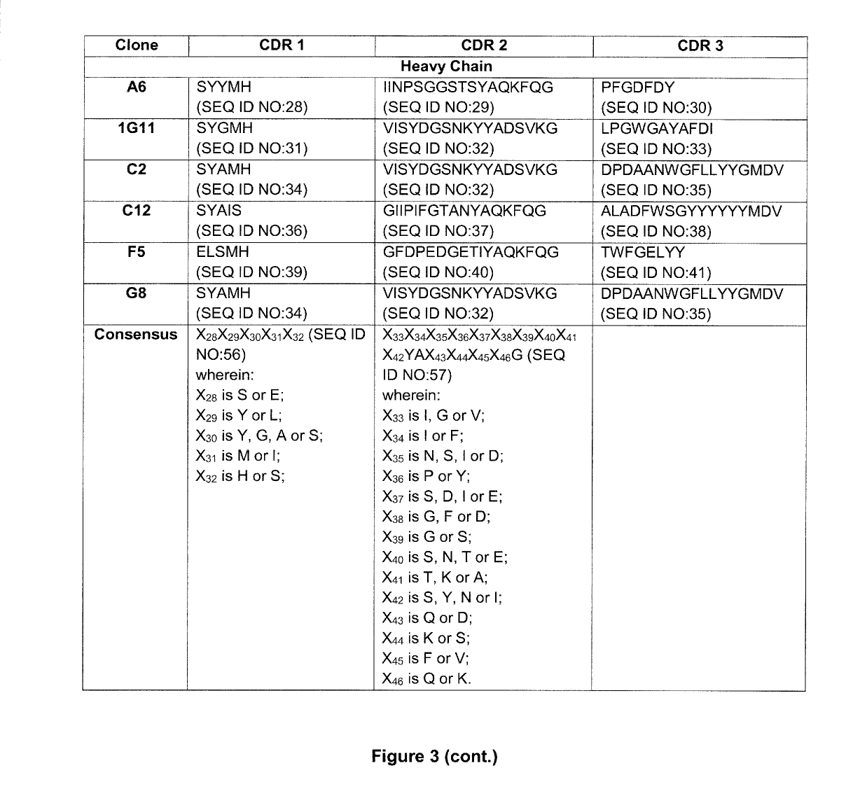

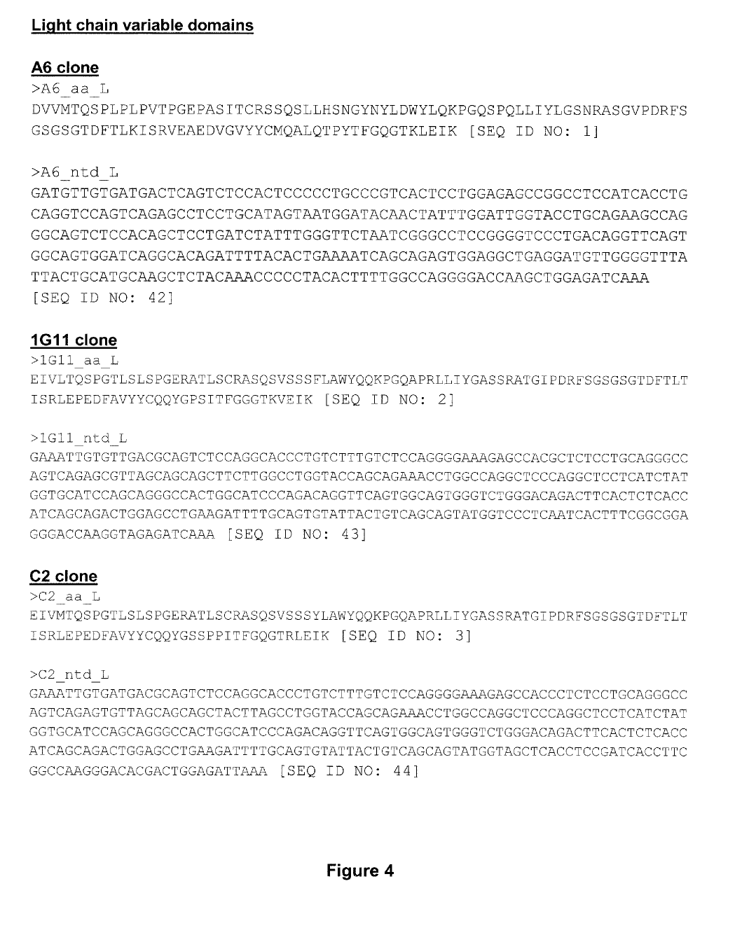

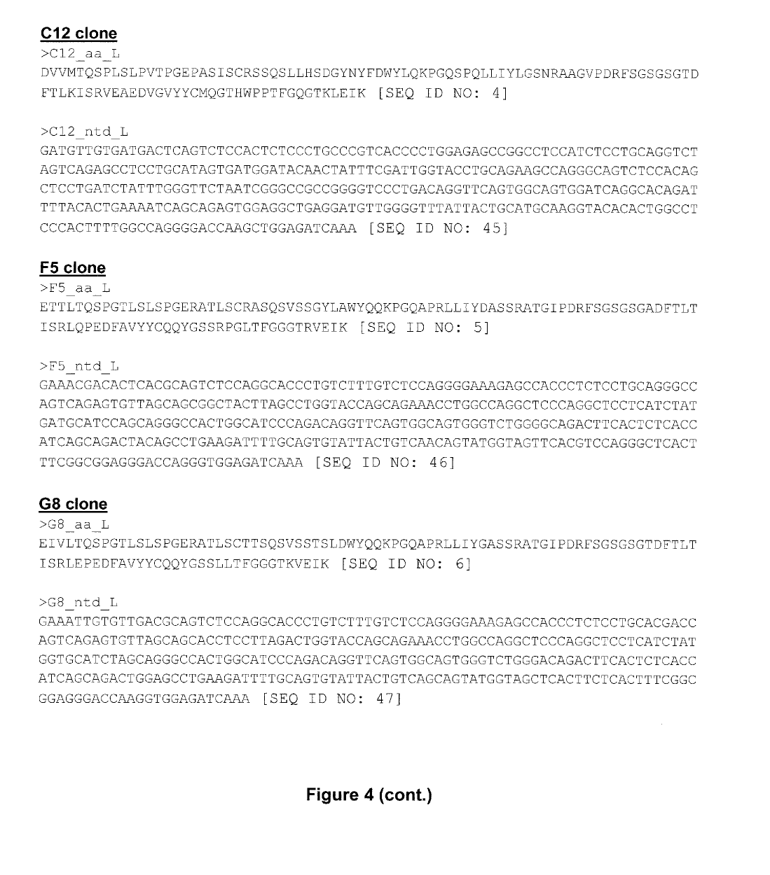

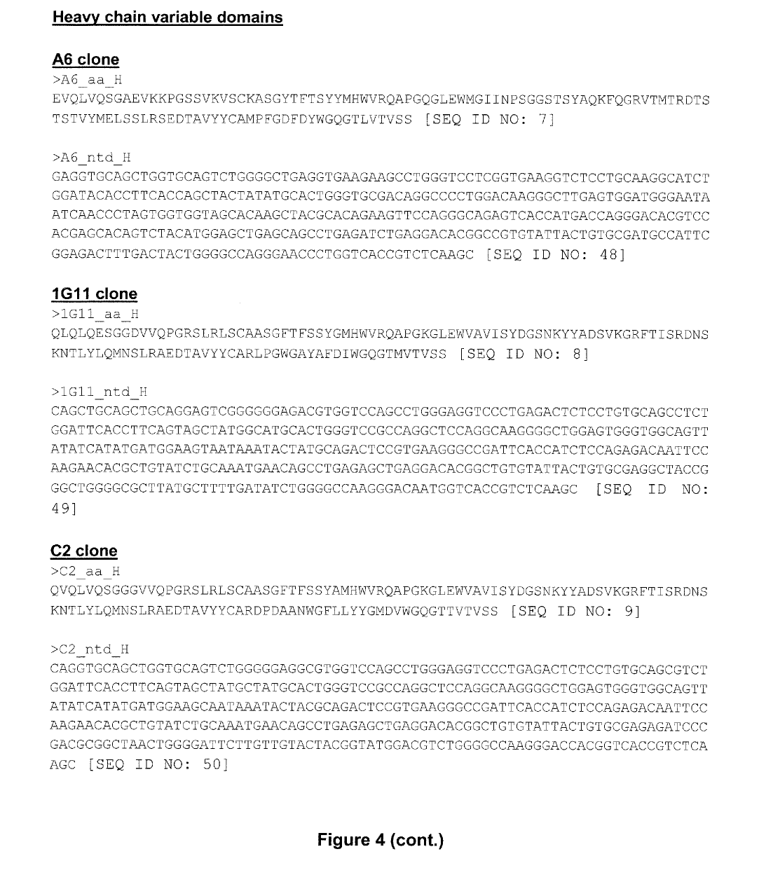

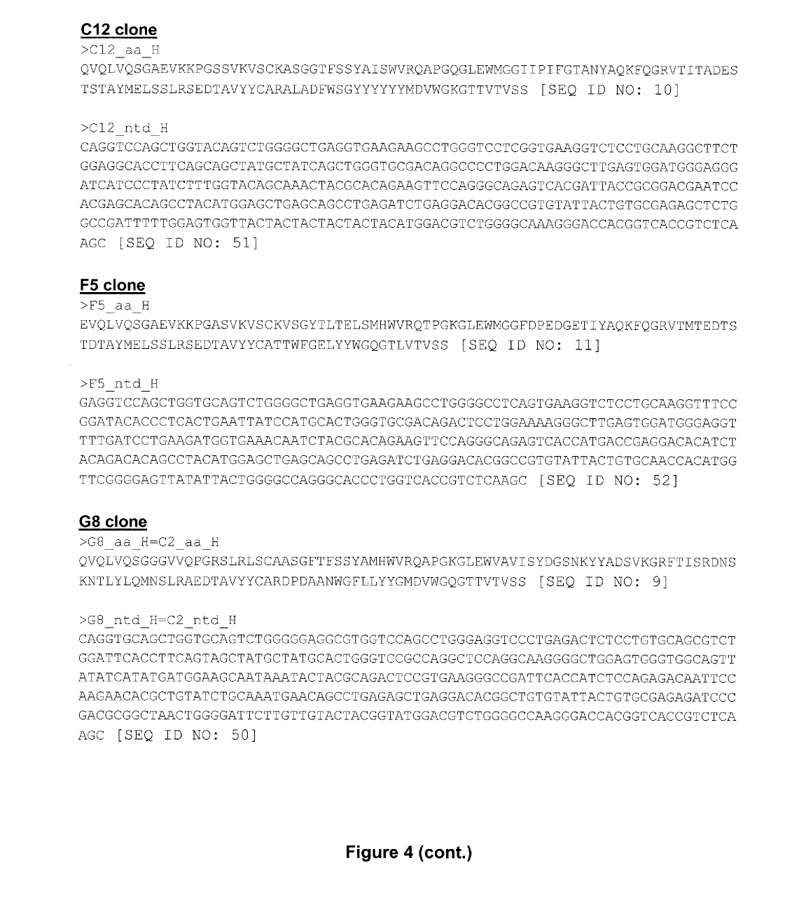

31. An isolated heavy chain variable region polypeptide comprising an amino acid sequence having at least 85% sequence identity to the heavy chain sequence of SEQ ID NO:7, 8, 9, 10 or 11 (FIG. 2).

32. An isolated light chain variable region polypeptide of any one of claims 23 to 27 in combination with a heavy chain variable region polypeptide according to any one of claims 28 to 31.

33. An antibody or antigen binding fragment which is capable of binding to LAG-3, comprising a heavy chain and a light chain variable region sequence, wherein:
the light chain comprises a LC-CDR1, LC-CDR2, LC-CDR3, having at least 85% overall sequence identity to LC-CDR1: one of X₁X₂SQSX₃X₄X₅X₆X₇X₈X₉X₁₀X₁₁X₁₂X₁₃ (SEQ ID NO:53), RASQSVSSGYLA (SEQ ID NO:23), RSSQSLLHSNGYNYLD (SEQ ID NO:12), RASQSVSSSFLA (SEQ ID NO:15), RASQSVSSSYLA (SEQ ID NO:18), RSSQSLLHSDGYNYFD (SEQ ID NO:20) or TTSQSVSSTSLD (SEQ ID NO:26), LC-CDR2: one of X₁₄X₁₅SX₁₆RAX₁₇ (SEQ ID NO:54), DASSRAT (SEQ ID NO:24), LGSNRAS (SEQ ID NO:13), GASSRAT (SEQ ID NO:16), or LGSNRAA (SEQ ID NO:21), LC-CDR3: one of X₁₈QX₁₈X₂₀X₂₁X₂₂X₂₃X₂₄X₂₅X₂₆X₂₇ (SEQ ID NO:55), QQYGSSRPGLT (SEQ ID NO:25), MQALQTPYT (SEQ ID NO:14), QQYGPSIT (SEQ ID NO:17), QQYGSSPPIT (SEQ ID NO:19), MQGTHWPPT (SEQ ID NO:22), or QQYGSSLLT (SEQ ID NO:27), respectively, where X₁=R or T; X₂=S, A or T; X₃=L or V; X₄=L or S; X₅=H or S; X₆=S, G or T; X₇=N, F, Y, D or S; X₈=G or L; X₉=Y, A or D; X₁₀=absent or N; X₁₁=absent or Y; X₁₂=absent, L or F; X₁₃=absent or D; X₁₄=L, G or D; X₁₅=G or A; X₁₆=N or S; X₁₇=S, T or A; X₁₈=M or Q; X₁₉=A, Y or G; X₂₀=L, G or T; X₂₁=Q, P, S or H; X₂₂=T, S or W; X₂₃=P, I, R or L; X₂₄=Y, T, P or L; X₂₅=absent, T, I or G; X₂₆=absent, T or L; and X₂₇=absent or T, and;
the heavy chain comprises a HC-CDR1, HC-CDR2, HC-CDR3, having at least 85% overall sequence identity to HC-CDR1: one of X₂₈X₂₉X₃₀X₃₁X₃₂ (SEQ ID NO:56), ELSMH (SEQ ID NO:39), SYYMH (SEQ ID NO:28), SYGMH (SEQ ID NO:31), SYAMH (SEQ ID NO:34), or SYAIS (SEQ ID NO:36), HC-CDR2: one of X₃₃X₃₄X₃₅X₃₆X₃₇X₃₈X₃₉X₄₀X₄₁X₄₂YAX₄₃X₄₄X₄₅X₄₆G (SEQ ID NO:57), GFDPEDGETIYAQKFQG (SEQ ID NO:40), IINPSGGSTSYAQKFQG (SEQ ID NO:29) VISYDGSNKYYADSVKG (SEQ ID NO:32), or GIIPIFGTANYAQKFQG (SEQ ID NO:37), HC-CDR3: one of TWFGELYY (SEQ ID NO:41), PFGDFDY (SEQ ID NO:30), LPGWGAYAFDI (SEQ ID NO:33), DPDAANWGFLLYYGMDV (SEQ ID NO:35), or ALADFWSGYYYYYYMDV (SEQ ID NO:38), respectively, where X₂₈=S or E; X₂₉=Y or L; X₃₀=Y, G, A or S; X₃₁=M or I; X₃₂=H or S; X₃₃=I, G or V; X₃₄=I or F; X₃₅=N, S, I or D; X₃₆=P or Y; X₃₇=S, D, I or E; X₃₈=G, F or D; X₃₉=G or S; X₄₀=S, N, T or E; X₄₁=T, K or A; X₄₂=S, Y, N or I; X₄₃=Q or D; X₄₄=K or S; X₄₅=F or V; and X₄₆ is Q or K.

34. An antibody or antigen binding fragment which is capable of binding to LAG-3, optionally isolated, comprising a heavy chain and a light chain variable region sequence, wherein:
the light chain sequence has at least 85% sequence identity to the light chain sequence: SEQ ID NO:1, 2, 3, 4, 5 or 6 (FIG. 1), and;
the heavy chain sequence has at least 85% sequence identity to the heavy chain sequence of SEQ ID NO:7, 8, 9, 10 or 11 (FIG. 2).

35. An antibody or antigen binding fragment, optionally isolated, which is capable of binding to LAG-3, which is a bispecific antibody or a bispecific antigen binding fragment comprising (i) an antigen binding fragment or polypeptide according to any one of claims 1 to 34, and (ii) an antigen binding fragment or polypeptide which is capable of binding to a target protein other than LAG-3.

36. The antibody, or antigen binding fragment, of claim 35, wherein the antigen binding fragment or polypeptide which is capable of binding to a target protein other than LAG-3 is capable of binding to one of PD-1, PD-L1, CD27, CD28, ICOS, CD40, CD122, OX43, 4-1BB, GITR, B7-H3, B7-H4, BTLA, CTLA-4, A2AR, VISTA, TIM-3, KIR, HER-2, HER-3, EGFR, EpCAM, CD30, CD33, CD38, CD20, CD24, CD90, CD15, CD52, CA-125, CD34, CA-15-3, CA-19-9, CEA, CD99, CD117, CD31, CD44, CD123, CD133, ABCB5 and CD45.

37. A chimeric antigen receptor (CAR) comprising an antigen binding fragment according to any one of claims 1 to 36.

38. A cell comprising the CAR according to claim 37.

39. An in vitro complex, optionally isolated, comprising an antibody, antigen binding fragment, polypeptide, CAR or cell according to any one of claims 1 to 38 bound to LAG-3.

40. A composition comprising the antibody, or antigen binding fragment, polypeptide, CAR or cell of any one of claims 1 to 37 and at least one pharmaceutically-acceptable carrier.

41. An isolated nucleic acid encoding the antibody, or antigen binding fragment, polypeptide or CAR of any of one of claims 1 to 37.

42. A vector comprising the nucleic acid of claim 41.

43. A host cell comprising the vector of claim 42.

44. A method for making an antibody, antigen binding fragment, polypeptide or CAR of any of one of claims 1 to 37 comprising culturing the host cell of claim 43 under conditions suitable for the expression of a vector encoding the antibody, or antigen binding fragment, polypeptide or CAR, and recovering the antibody, or antigen binding fragment, polypeptide or CAR.

45. An antibody, antigen binding fragment, polypeptide, CAR, cell or composition according to any one of claim 1 to 38 or 40 for use in therapy, or in a method of medical treatment.

46. An antibody, antigen binding fragment, polypeptide, CAR, cell or composition according to any one of claim 1 to 38 or 40 for use in the treatment of a T-cell dysfunctional disorder.

47. An antibody, antigen binding fragment, polypeptide, CAR, cell or composition according to any one of claim 1 to 38 or 40 for use in the treatment of cancer.

48. An antibody, antigen binding fragment, polypeptide, CAR, cell or composition according to any one of claim 1 to 38 or 40 for use in the treatment of an infectious disease.

49. Use of an antibody, antigen binding fragment, polypeptide, CAR, cell or composition according to any one of claim 1 to 38 or 40 in the manufacture of a medicament for use in the treatment of a T-cell dysfunctional disorder.

50. Use of an antibody, antigen binding fragment, polypeptide, CAR, cell or composition according to any one of claim 1 to 38 or 40 in the manufacture of a medicament for use in the treatment of cancer.

51. Use of an antibody, antigen binding fragment, polypeptide, CAR, cell or composition according to any one of claim 1 to 38 or 40 in the manufacture of a medicament for use in the treatment of an infectious disease.

52. A method, in vitro or in vivo, of enhancing T-cell function comprising administering an antibody, antigen binding fragment, polypeptide, CAR, cell or composition according to any one of claim 1 to 38 or 40 to a dysfunctional T-cell.

53. A method of treating a T-cell dysfunctional disorder comprising administering an antibody, antigen binding fragment, polypeptide, CAR, cell or composition according to any one of claim 1 to 38 or 40 to a patient suffering from a T-cell dysfunctional disorder.

54. A method of treating cancer comprising administering an antibody, antigen binding fragment, polypeptide, CAR, cell or composition according to any one of claim 1 to 38 or 40 to a patient suffering from a cancer.

55. A method of treating an infectious disease comprising administering an antibody, antigen binding fragment, polypeptide, CAR, cell or composition according to any one of claim 1 to 38 or 40 to a patient suffering from an infectious disease.

56. A method comprising contacting a sample containing, or suspected to contain, LAG-3 with an antibody, antigen binding fragment, CAR or cell according to any one of claims 1 to 38 and detecting the formation of a complex of antibody, antigen binding fragment, CAR or cell, and LAG-3.

57. A method of diagnosing a disease or condition in a subject, the method comprising contacting, in vitro, a sample from the subject with an antibody, antigen binding fragment, CAR or cell according to any one of claims 1 to 38 and detecting the formation of a complex of antibody, or antigen binding fragment, CAR or cell and LAG-3.

58. A method of selecting or stratifying a subject for treatment with LAG-3 or MHC class II targeted agents, the method comprising contacting, in vitro, a sample from the subject with an antibody, antigen binding fragment, CAR or cell according to any one of claims 1 to 38 and detecting the formation of a complex of antibody, or antigen binding fragment, CAR or cell and LAG-3.

59. Use of an antibody, antigen binding fragment, CAR or cell according to any one of claims 1 to 38 for the detection of LAG-3 in vitro.

60. Use of an antibody, antigen binding fragment, CAR or cell according to any one of claims 1 to 38 as an in vitro diagnostic agent.

61. A method for expanding a population of T cells, wherein T cells are contacted in vitro or ex vivo with an antibody, antigen binding fragment, polypeptide, CAR, cell or composition according to any one of claim 1 to 38 or 40.

62. A method of treatment of a subject having a T-cell dysfunctional disorder, the method comprising culturing T cells obtained from a blood sample from a subject in the presence of an antibody, antigen binding fragment, polypeptide, CAR, cell or composition according to any one of claim 1 to 38 or 40 so as to expand the T cell population, collecting expanded T cells, and administering the expanded T cells to a subject in need of treatment.

63. A method of treating or preventing a cancer in a subject, comprising:
(a) isolating at least one cell from a subject;
(b) modifying the at least one cell to express or comprise the antibody, antigen binding fragment, polypeptide, CAR, nucleic acid or vector according to any one of claim 1 to 37, 41 or 42, and;
(c) administering the modified at least one cell to a subject.

64. A method of treating or preventing a cancer in a subject, comprising:
(a) isolating at least one cell from a subject;
(b) introducing into the at least one cell the nucleic acid according to claim 41 or the vector according to claim 42, thereby modifying the at least one cell, and;
(c) administering the modified at least one cell to a subject.

65. A kit of parts comprising a predetermined quantity of the antibody, antigen binding fragment, polypeptide, CAR, composition, nucleic acid, vector or cell according to any one of claims 1 to 38, or 40 to 43.
